(19) United States
(12) Patent Application Publication
(10) Pub. No.: US 2004/0202603 A1
(43) Pub. Date: Oct. 14, 2004

(54) Functionalized nanotubes

(75) Inventors: Alan Fischer, Cambridge, MA (US); Robert Hoch, Hensonville, NY (US); David Moy, Winchester, MA (US); Ming Lu, Lanham, MD (US); Mark Martin, N. Bethesda, MD (US); Chun Ming Niu, Somerville, MA (US); Naoya Ogata, Tokyo (JP); Howard Tennent, Kennett Square, PA (US); Liwen Dong, Rockville, MD (US); Ji Sun, Potomac, MD (US); Larry Helms, Germantown, MD (US); Fabian Jameison, Gaithersburg, MD (US); Pam Liang, Alhambra, CA (US); David Simpson, N. Bethesda, MD (US)

Correspondence Address: KRAMER LEVIN NAFTALIS & FRANKEL LLP, INTELLECTUAL PROPERTY DEPARTMENT, 919 THIRD AVENUE, NEW YORK, NY, 10022, US

(73) Assignee: Hyperion Catalysis International, Inc.

(21) Appl. No.: 10/837,125

(22) Filed: Apr. 30, 2004

Related U.S. Application Data
(63) Continuation of application No. 08/812,856, filed on Mar. 6, 1997, now abandoned.
Application No. 08/812,856 is a continuation of application No. 08/611,368, filed on Mar. 6, 1996, now abandoned.
(63) Continuation-in-part of application No. 09/594,673, filed on Jun. 16, 2000.
Application No. 09/594,673 is a division of application No. 08/352,400, filed on Dec. 8, 1994, now Pat. No. 6,203,814.
(60) Provisional application No. 60/037,238, filed on Sep. 25, 1996.

(51) Int. Cl. D01F 9/12; C07C 63/333

(52) U.S. Cl. 423/447.2; 562/492; 564/426

(57) ABSTRACT

Graphitic nanotubes, which includes tubular fullerenes (commonly called “buckytubes”) and fibrils, which are functionalized by chemical substitution or by adsorption of functional moieties. More specifically the invention relates to graphitic nanotubes which are uniformly or non-uniformly substituted with chemical moieties or upon which certain cyclic compounds are adsorbed and to complex structures comprised of such functionalized nanotubes linked to one another. The invention also relates to methods for introducing functional groups onto the surface of such nanotubes. The invention further relates to uses for functionalized nanotubes.

### CROSS-REFERENCE TO RELATED APPLICATION

This application is a continuation-in-part of U.S. application Ser. No. 08/352,400, filed Dec. 8, 1994, the contents of which are hereby incorporated by reference.

### FIELD OF THE INVENTION

The invention relates broadly to graphitic nanotubes, which includes tubular fullerenes (commonly called “buckytubes”) and fibrils, which are functionalized by chemical substitution or by adsorption of functional moieties. More specifically the invention relates to graphitic nanotubes which are uniformly or non-uniformly substituted with chemical moieties or upon which certain cyclic compounds are adsorbed and to complex structures comprised of such functionalized fibrils linked to one another. The invention also relates to methods of introducing functional groups onto the surface of such fibrils.

### BACKGROUND OF THE INVENTION

This invention lies in the field of submicron graphitic fibrils, sometimes called vapor grown carbon fibers. Carbon fibrils are vermicular carbon deposits having diameters less than 1.0μ, preferably less than 0.5μ, and even more preferably less than 0.2μ. They exist in a variety of forms and have been prepared through the catalytic decomposition of various carbon-containing gases at metal surfaces. Such vermicular carbon deposits have been observed almost since the advent of electron microscopy. A good early survey and reference is found in Baker and Harris, Chemistry and Physics of Carbon, Walker and Thrower ed., Vol. 14, 1978, p. 83, hereby incorporated by reference. See also, Rodriguez, N., J. Mater. Research, Vol. 8, p. 3233 (1993), hereby incorporated by reference.

In 1976, Endo et al. (see Obelin, A. and Endo, M., J. of Crystal Growth, Vol. 32 (1976), pp. 335-349, hereby incorporated by reference) elucidated the basic mechanism by which such carbon fibrils grow. There were seen to originate from a metal catalyst particle, which, in the presence of a hydrocarbon containing gas, becomes supersaturated in carbon. A cylindrical ordered graphitic core is extruded which immediately, according to Endo et al., becomes coated with an outer layer of pyrolytically deposited graphite. These fibrils with a pyrolytic overcoat typically have diameters in excess of 0.1 Å, more typically 0.2 to 0.5μ.

In 1983, Tennent, U.S. Pat. No. 4,663,230, hereby incorporated by reference, succeeded in growing cylindrical ordered graphite cores, uncontaminated with pyrolytic carbon. Thus, the Tennent invention provided access to smaller diameter fibrils, typically 35 to 700 Å (0.0035 to 0.0701μ) and to an ordered, “as grown” graphitic surface. Fibrillar carbons of less perfect structure, but also without a pyrolytic carbon outer layer have also been grown.

The fibrils, buckytubes and nanofibers that are functionalized in this application are distinguishable from continuous carbon fibers commercially available as reinforcement materials. In contrast to fibrils, which have, desirably large, but unavoidably finite aspect ratios, continuous carbon fibers have aspect ratios (L/D) of at least 104 and often 106 or more. The diameter of continuous fibers is also far larger than that of fibrils, being always >1.0 and typically 5 to 7μ.

Continuous carbon fibers are made by the pyrolysis of organic precursor fibers, usually rayon, polyacrylonitrile (PAN) and pitch. Thus, they may include heteroatoms within their structure. The graphitic nature of “as made” continuous carbon fibers varies, but they may be subjected to a subsequent graphitization step. Differences in degree of graphitization, orientation and crystallinity of graphite planes, if they are present, the potential presence of heteroatoms and even the absolute difference in substrate diameter make experience with continuous fibers poor predictors of nanofiber chemistry.

Tennent, U.S. Pat. No. 4,663,230 describes carbon fibrils that are free of a continuous thermal carbon overcoat and have multiple graphitic outer layers that are substantially parallel to the fibril axis. As such they may be characterized as having their c-axes, the axes which are perpendicular to the tangents of the curved layers of graphite, substantially perpendicular to their cylindrical axes. They generally have diameters no greater than 0.1μ and length to diameter ratios of at least 5. Desirably they are substantially free of a continuous thermal carbon overcoat, i.e., pyrolytically deposited carbon resulting from thermal cracking of the gas feed used to prepare them.

Tennent, et al., U.S. Pat. No. 5,171,560, hereby incorporated by reference, describes carbon fibrils free of thermal overcoat and having graphitic layers substantially parallel to the fibril axes such that the projection of said layers on said fibril axes extends for a distance of at least two fibril diameters. Typically, such fibrils are substantially cylindrical, graphitic nanotubes of substantially constant diameter and comprise cylindrical graphitic sheets whose c-axes are substantially perpendicular to their cylindrical axis. They are substantially free of pyrolytically deposited carbon, have a diameter less than 0.1μ and a length to diameter ratio of greater than 5. These fibrils are of primary interest in the invention.

Further details regarding the formation of carbon fibril aggregates may be found in the disclosure of Snyder et al., U.S. patent application Ser. No. 149,573, filed Jan. 28, 1988, and PCT Application No. US89/00322, filed Jan. 28, 1989 (“Carbon Fibrils”) WO 89/07163, and Moy et al., U.S. patent application Ser. No. 413,837 filed Sep. 28, 1989 and PCT Application No. US90/05498, filed Sep. 27, 1990 (“Fibril Aggregates and Method of Making Same”) WO 91/05089, all of which are assigned to the same assignee as the invention here and are hereby incorporated by reference.

Moy et al., U.S. Ser. No. 07/887,307 filed May 22, 1992, hereby incorporated by reference, describes fibrils prepared as aggregates having various macroscopic morphologies (as determined by scanning electron microscopy) in which they are randomly entangled with each other to form entangled balls of fibrils resembling bird nests (“BN”); or as aggregates consisting of bundles of straight to slightly bent or kinked carbon fibrils having substantially the same relative orientation, and having the appearance of combed yarn (“CY”) e.g., the longitudinal axis of each fibril (despite individual bends or kinks) extends in the same direction as that of the surrounding fibrils in the bundles; or as aggregates consisting of straight to slightly bent or kinked fibrils which are loosely entangled with each other to form an “open net” (“ON”) structure. In open net structures the degree of fibril entanglement is greater than observed in the combed yarn aggregates (in which the individual fibrils have substantially the same relative orientation) but less than that of bird nests. CY and ON aggregates are more readily dispersed than BN making them useful in composite fabrication where uniform properties throughout the structure are desired.

When the projection of the graphitic layers on the fibril axis extends for a distance of less than two fibril diameters, the carbon planes of the graphitic nanofiber, in cross section, take on a herring bone appearance. These are termed fishbone fibrils. Geus, U.S. Pat. No. 4,855,091, hereby incorporated by reference, provides a procedure for preparation of fishbone fibrils substantially free of a pyrolytic overcoat. These fibrils are also useful in the practice of the invention.

Carbon nanotubes of a morphology similar to the catalytically grown fibrils described above have been grown in a high temperature carbon arc (Iijima, Nature 354 56 1991). It is now generally accepted (Weaver, Science 265 1994) that these arc-grown nanofibers have the same morphology as the earlier catalytically grown fibrils of Tennent. Arc grown carbon nanofibers are also useful in the invention.

McCarthy et al., U.S. patent application Ser. No. 351,967 filed May 15, 1989, hereby incorporated by reference, describes processes for oxidizing the surface of carbon fibrils that include contacting the fibrils with an oxidizing agent that includes sulfuric acid (H₂SO₄) and potassium chlorate (KClO₃) under reaction conditions (e.g., time, temperature, and pressure) sufficient to oxidize the surface of the fibril. The fibrils oxidized according to the processes of McCarthy, et al. are non-uniformly oxidized, that is, the carbon atoms are substituted with a mixture of carboxyl, aldehyde, ketone, phenolic and other carbonyl groups.

Fibrils have also been oxidized non-uniformly by treatment with nitric acid. International Application PCT/US94/10168 discloses the formation of oxidized fibrils containing a mixture of functional groups. Hoogenvaad, M. S., et al. (“Metal Catalysts supported on a Novel Carbon Support”, Presented at Sixth International Conference on Scientific Basis for the Preparation of Heterogeneous Catalysts, Brussels, Belgium, September 1994) also found it beneficial in the preparation of fibril-supported precious metals to first oxidize the fibril surface with nitric acid. Such pretreatment with acid is a standard step in the preparation of carbon-supported noble metal catalysts, where, given the usual sources of such carbon, it serves as much to clean the surface of undesirable materials as to functionalize it.

In published work, McCarthy and Bening (Polymer Preprints ACS Div. of Polymer Chem. 30 (1)420(1990)) prepared derivatives of oxidized fibrils in order to demonstrate that the surface comprised a variety of oxidized groups. The compounds they prepared, phenylhydrazones, haloaromaticesters, thallous salts, etc., were selected because of their analytical utility, being, for example, brightly colored, or exhibiting some other strong and easily identified and differentiated signal. These compounds were not isolated and are, unlike the derivatives described herein, of no practical significance.

While many uses have been found for carbon fibrils and aggregates of carbon fibrils, as described in the patents and patent applications referred to above, many different and important uses may be developed if the fibril surfaces are functionalized. Functionalization, either uniformly or non-uniformly, permits interaction of the functionalized fibrils with various substrates to form unique compositions of matter with unique properties and permits fibril structures to be created based on linkages between the functional sites on the fibrils' surfaces.

### OBJECTS OF THE INVENTION

It is therefore a primary object of this invention to provide functionalized fibrils, i.e. fibrils whose surfaces are uniformly or non-uniformly modified so as to have a functional chemical moiety associated therewith.

It is a further and related object of this invention to provide fibrils whose surfaces are functionalized by reaction with oxidizing or other chemical media.

It is a further and related object of this invention to provide fibrils whose surfaces are uniformly modified either by chemical reaction or by physical adsorption of species which themselves have a chemical reactivity.

It is a further object to provide fibrils whose surfaces have been modified e.g. by oxidation which are then further modified by reaction with functional groups.

It is still a further and related object of this invention to provide fibrils whose surfaces are modified with a spectrum of functional groups so that the fibrils can be chemically reacted or physically bonded to chemical groups in a variety of substrates.

It is still the further and related object of this invention to provide complex structures of fibrils by linking functional groups on the fibrils with one another by a range of linker chemistries.

It is still a further and related object of this invention to provide methods for chemical modification of fibril surfaces and methods for physically absorbing species on the surfaces of fibrils so as to provide, in each case, a functional moiety associated with the surface of the fibril.

It is yet a further object of this invention to provide new compositions of matter based upon the functionalized fibrils.

### BRIEF DESCRIPTION OF THE DRAWINGS

FIG. 1 is a graphical representation of an assay of BSA binding to plain fibrils, carboxy fibrils, and PEG-modified fibrils.

FIG. 2 is a graphical representation of an assay of β-lactoglobulin binding to carboxy fibrils and PEG-modified fibrils prepared by two different methods.

FIG. 3 is a graphical representation of the elution profile of bovine serum albumin (BSA) on a tertiary amine fibril column.

FIG. 4 is a graphical representation of the elution profile of BSA on a quaternary amine fibril column.

FIG. 5 is the reaction sequence for the preparation of lysine-based dendrimeric fibrils.

FIG. 6 is a graphical representation of cyclic voltammograms demonstrating the use of iron phthalocyanine modified fibrils in a flow cell.

FIG. 7 is the reaction sequence for the preparation of bifunctional fibrils by the addition of NE-(tert-butoxycarbonyl)-L-lysine.

FIG. 8 is a graphical representation of the results of the synthesis of ethyl butyrate using fibril-immobilized lipase.

FIG. 9 is a graphical representation of the results of separation of alkaline phosphatase (AP) from a mixture of AP and β-galactosidase (βG) using AP inhibitor-modified fibrils.

FIG. 10 is a graphical representation of the results of separation of βG from a mixture of AP and βG using βG-modified fibrils.

### DETAILED DESCRIPTION OF THE INVENTION

The invention is directed to compositions which broadly have the formula

[Rₘ

where n is an integer, L is a number less than 0.1 n, m is a number less than 0.5 n,

each of R is the same and is selected from SO₃H, COOH, NH₂, OH, R′CHOH, CHO, CN, COCl, halide, COSH, SH, COOR′, SR′, SiR′₃, Si(OR′)_{y}R′_{3-y}, Si(O—SiR′₂)OR′, R″, Li, AlR′₂, Hg—X, TlZ₂ and Mg—X,

y is an integer equal to or less than 3,

R′ is hydrogen, alkyl, aryl, cycloalkyl, or aralkyl, cycloaryl, or poly(alkylether),

R″ is fluoroalkyl, fluoroaryl, fluorocycloalkyl, fluoroaralkyl or cycloaryl,

X is halide, and

Z is carboxylate or trifluoroacetate.

The carbon atoms, Cₙ, are surface carbons of a substantially cylindrical, graphitic nanotube of substantially constant diameter. The nanotubes include those having a length to diameter ratio of greater than 5 and a diameter of less than 0.5μ, preferably less than 0.1. The nanotubes can also be substantially cylindrical, graphitic nanotubes which are substantially free of pyrolytically deposited carbon, more preferably those characterized by having a projection of the graphite layers on the fibril axis which extends for a distance of at least two fibril diameters and/or those having cylindrical graphitic sheets whose c-axes are substantially perpendicular to their cylindrical axis. These compositions are uniform in that each of R is the same.

Non-uniformly substituted nanotubes are also prepared. These include compositions of the formula

[Rₘ

where n, L, m, R and the nanotube itself are as defined above, provided that each of R does not contain oxygen, or, if each of R is an oxygen-containing group COOH is not present.

Functionalized nanotubes having the formula

[Rₘ

where n, L, m, R and R′ have the same meaning as above and the carbon atoms are surface carbon atoms of a fishbone fibril having a length to diameter ratio greater than 5, are also included within the invention. These may be uniformly or non-uniformly substituted. Preferably, the nanotubes are free of thermal overcoat and have diameters less than 0.5μ.

Also included in the invention are functionalized nanotubes having the formula

[[R′—R]ₘ

where n, L, m, R′ and R have the same meaning as above. The carbon atoms, Cₙ, are surface carbons of a substantially cylindrical, graphitic nanotube of substantially constant diameter. The nanotubes have a length to diameter ratio of greater than 5 and a diameter of less than 0.5μ, preferably less than 0.1. The nanotubes may be nanotubes which are substantially free of pyrolytically deposited carbon. More preferably, the nanotubes are those in which the projection of the graphite layers on the fibril axes extends for a distance of at least two fibril diameters and/or those having cylindrical graphitic sheets whose c-axes are substantially perpendicular to their cylindrical axis.

In both uniformly and non-uniformly substituted nanotubes, the surface atoms Cₙ are reacted. Most carbon atoms in the surface layer of a graphitic fibril, as in graphite, are basal plane carbons. Basal plane carbons are relatively inert to chemical attack. At defect sites, where, for example, the graphitic plane fails to extend fully around the fibril, there are carbon atoms analogous to the edge carbon atoms of a graphite plane (See Urry, Elementary Equilibrium Chemistry of Carbon, Wiley, New York 1989.) for a discussion of edge and basal plane carbons).

At defect sites, edge or basal plane carbons of lower, interior layers of the nanotube may be exposed. The term surface carbon includes all the carbons, basal plane and edge, of the outermost layer of the nanotube, as well as carbons, both basal plane and/or edge, of lower layers that may be exposed at defect sites of the outermost layer. The edge carbons are reactive and must contain some heteroatom or group to satisfy carbon valency.

The substituted nanotubes described above may advantageously be further functionalized. Such compositions include compositions of the formula

[Aₘ

where the carbons are surface carbons of a nanotube, n, L and m are as described above,

A is selected from

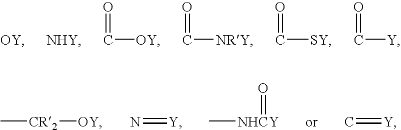

Y is an appropriate functional group of a protein, a peptide, an amino acid, an enzyme, an antibody, a nucleotide, an oligonucleotide, an antigen, or an enzyme substrate, enzyme inhibitor or the transition state analog of an enzyme substrate or is selected from R′—OH, R′—NR′₂, R′SH, R′CHO, R′CN, R′X, R′N⁺(R′)₃X⁻, R′SiR′₃, R′Si(OR′)_{y}R′_{3-y}R′Si)O—SiR′₂)OR′, R′—R″, R′—N—CO, (C₂H₄O)_{w}, (C₃H₆O)_{w}H, (C₂H₄O)_{w}—R′, (C₃H₆O)_{w}—R′,

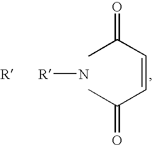

and w is an integer greater than one and less than 200. The carbon atoms, Cₙ, are surface carbons of a substantially cylindrical, graphitic nanotube of substantially constant diameter. The nanotubes include those having a length to diameter ratio of greater than 5 and a diameter of less than 0.1μ, preferably less than 0.051μ. The nanotubes can also be substantially cylindrical, graphitic nanotubes which are substantially free of pyrolytically deposited carbon. More preferably they are characterized by having a projection of the graphite layers on the fibril axes which extends for a distance of at least two fibril diameters and/or they are comprised of cylindrical graphitic sheets whose c-axes are substantially perpendicular to their cylindrical axes. Preferably, the nanotubes are free of thermal overcoat and have diameters less than 0.5μ.

The functional nanotubes of structure

[[R′—R]ₘ

may also be functionalized to produce compositions having the formula

[[R′-A]ₘ

where n, L, m, R′ and A are as defined above. The carbon atoms, Cₙ, are surface carbons of a substantially cylindrical, graphitic nanotube of substantially constant diameter. The nanotubes include those having a length to diameter ratio of greater than 5 and a diameter of less than 0.51, preferably less than 0.1 g. The nanotubes can also be substantially cylindrical, graphitic nanotubes which are substantially free of pyrolytically deposited carbon. More preferably they are characterized by having a projection of the graphite layers on the fibril axes which extends for a distance of at least two fibril diameters and/or by having cylindrical graphitic sheets whose c-axes are substantially perpendicular to their cylindrical axis. Preferably, the nanotubes are free of thermal overcoat and have diameters less than 0.5μ.

The compositions of the invention also include nanotubes upon which certain cyclic compounds are adsorbed. These include compositions of matter of the formula

[[X—Rₐ]ₘ

where n is an integer, L is a number less than 0.1 n, m is less than 0.5 n, a is zero or a number less than 10, X is a polynuclear aromatic, polyheteronuclear aromatic or metallopolyheteronuclear aromatic moiety and R is as recited above. The carbon atoms, Cₙ, are surface carbons of a substantially cylindrical, graphitic nanotube of substantially constant diameter. The nanotubes include those having a length to diameter ratio of greater than 5 and a diameter of less than 0.5μ, preferably less than 0.1μ. The nanotubes can also be substantially cylindrical, graphitic nanotubes which are substantially free of pyrolytically deposited carbon and more preferably those characterized by having a projection of the graphite layers on said fibril axes which extend for a distance of at least two fibril diameters and/or those having cylindrical graphitic sheets whose c-axes are substantially perpendicular to their cylindrical axes. Preferably, the nanotubes are free of thermal overcoat and have diameters less than 0.5μ.

Preferred cyclic compounds are planar macrocycles as described on p. 76 of Cotton and Wilkinson, Advanced Organic Chemistry. More preferred cyclic compounds for adsorption are porphyrins and phthalocyanines.

The adsorbed cyclic compounds may be functionalized. Such compositions include compounds of the formula

[[X-Aₐ]m

where m, n, L, a, X and A are as defined above and the carbons are surface carbons of a substantially cylindrical graphitic nanotube as described above.

The carbon fibrils functionalized as described above may be incorporated in a matrix. Preferably, the matrix is an organic polymer (e.g., a thermoset resin such as epoxy, bismaleimide, polyamide, or polyester resin; a thermoplastic resin; a reaction injection molded resin; or an elastomer such as natural rubber, styrene-butadiene rubber, or cis-1,4-polybutadiene); an inorganic polymer (e.g., a polymeric inorganic oxide such as glass), a metal (e.g., lead or copper), or a ceramic material (e.g., Portland cement). Beads may be formed from the matrix into which the fibrils have been incorporated. Alternately, functionalized fibrils can be attached to the outer surface of functionalized beads.

Without being bound to a particular theory, the functionalized fibrils are better dispersed into polymer systems because the modified surface properties are more compatible with the polymer, or, because the modified functional groups (particularly hydroxyl or amine groups) are bonded directly to the polymer as terminal groups. In this way, polymer systems such as polycarbonates, polyurethanes, polyesters or polyamides/imides bond directly to the fibrils making the fibrils easier to disperse with improved adherence.

The invention is also in methods of introducing functional groups onto the surface of carbon fibrils by contacting carbon fibrils with a strong oxidizing agent for a period of time sufficient to oxidize the surface of said fibrils and further contacting said fibrils with a reactant suitable for adding a functional group to the oxidized surface. In a preferred embodiment of the invention, the oxidizing agent is comprised of a solution of an alkali metal chlorate in a strong acid. In other embodiments of the invention the alkali metal chlorate is sodium chlorate or potassium chlorate. In preferred embodiments the strong acid used is sulfuric acid. Periods of time sufficient for oxidation are from about 0.5 hours to about 24 hours.

In a further preferred embodiment, a composition having the formula [[CH(R′)OH]ₘ, wherein n, L, R′ and m are as defined above, is formed by reacting R′CH₂OH with the surface carbons of a nanotube in the presence of a free radical initiator such as benzoyl peroxide.

The invention is also in a method for linking proteins to nanotubes modified by an NHS ester, by forming a covalent bond between the NHS ester and the amino group of the protein.

The invention is also in methods for producing a network of carbon fibrils comprising contacting carbon fibrils with an oxidizing agent for a period of time sufficient to oxidize the surface of the carbon fibrils, contacting the surface-oxidized carbon fibrils with reactant suitable for adding a functional group to the surface of the carbon fibrils, and further contacting the surface-functionalized fibrils with a cross-linking agent effective for producing a network of carbon fibrils. A preferred cross-linking agent is a polyol, polyamine or polycarboxylic acid.

Functionalized fibrils also are useful for preparing rigid networks of fibrils. A well-dispersed, three-dimensional network of acid-functionalized fibrils may, for example, be stabilized by cross-linking the acid groups (inter-fibril) with polyols or polyamines to form a rigid network.

The invention also includes three-dimensional networks formed by linking functionalized fibrils of the invention. These complexes include at least two functionalized fibrils linked by one or more linkers comprising a direct bond or chemical moiety. These networks comprise porous media of remarkably uniform equivalent pore size. They are useful as adsorbents, catalyst supports and separation media.

Although the interstices between these fibrils are irregular in both size and shape, they can be thought of as pores and characterized by the methods used to characterize porous media. The size of the interstices in such networks can be controlled by the concentration and level of dispersion of fibrils, and the concentration and chain lengths of the cross-linking agents. Such materials can act as structured catalyst supports and may be tailored to exclude or include molecules of a certain size. Aside from conventional industrial catalysis, they have special applications as large pore supports for biocatalysts.

The rigid networks can also serve as the backbone in biomimetic systems for molecular recognition. Such systems have been described in U.S. Pat. No. 5,110,833 and International Patent Publication No. WO93/19844. The appropriate choices for cross-linkers and complexing agents allow for stabilization of specific molecular frameworks.

### Methods of Functionalizing Nanotubes

The uniformly functionalized fibrils of the invention can be directly prepared by sulfonation, electrophilic addition to deoxygenated fibril surfaces or metallation. When arc grown nanofibers are used, they may require extensive purification prior to functionalization. Ebbesen et al. (Nature 367 519 (1994)) give a procedure for such purification.

Preferably, the carbon fibrils are processed prior to contacting them with the functionalizing agent. Such processing may include dispersing the fibrils in a solvent. In some instances the carbon fibrils may then be filtered and dried prior to further contact.

### 1. Sulfonation

Background techniques are described in March, J. P., Advanced Organic Chemistry, 3rd Ed. Wiley, New York 1985; House, H., Modern Synthetic Reactions, 2nd Ed., Benjamin/Cummings, Menlo Park, Calif. 1972.

Activated C—H (including aromatic C—H) bonds can be sulfonated using fuming sulfuric acid (oleum), which is a solution of conc. sulfuric acid containing up to 20% SO₃. The conventional method is via liquid phase at T-80° C. using oleum; however, activated C—H bonds can also be sulfonated using SO₃ in inert, aprotic solvents, or SO₃ in the vapor phase. The reaction is:

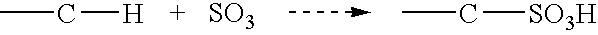

Over-reaction results in formation of sulfones, according to the reaction:

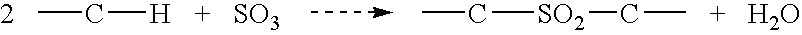

### EXAMPLE 1

### Activation of C—H Bonds Using Sulfuric Acid

Reactions were carried out in the gas phase and in solution without any significant difference in results. The vapor phase reaction was carried out in a horizontal quartz tube reactor heated by a Lindberg furnace. A multi-neck flask containing 20% SO₃ in conc. H₂SO₄ fitted with gas inlet/outlet tubes was used as the SO₃ source.

A weighed sample of fibrils (BN or CC) in a porcelain boat was placed in the 1″ tube fitted with a gas inlet; the outlet was connected to a conc. H₂SO₄ bubbler trap. Argon was flushed through the reactor for 20 min to remove all air, and the sample was heated to 300° C. for 1 hour to remove residual moisture. After drying, the temperature was adjusted to reaction temperature under argon.

When the desired temperature was stabilized, the SO₃ source was connected to the reactor tube and an argon stream was used to carry SO₃ vapors into the quartz tube reactor. Reaction was carried out for the desired time at the desired temperature, after which the reactor was cooled under flowing argon. The fibrils were then dried at 90° C. at 5″ Hg vacuum to obtain the dry weight gain. Sulfonic acid (—SO₃H) content was determined by reaction with 0.100N NaOH and back-titration with 0.100N HCl using pH 6.0 as the end point.

The liquid phase reaction was carried out in conc. sulfuric acid containing 20% SO₃ in a multi-neck 100 cc flask fitted with a thermometer/temperature controller and a magnetic stirrer. A fibril slurry in conc. H₂SO₄ (50) was placed in the flask. The oleum solution (20 cc) was preheated to −60° C. before addition to the reactor. After reaction, the acid slurry was poured onto cracked ice, and diluted immediately with 1 l DI water. The solids were filtered and washed exhaustively with DI water until there was no change in pH of the wash effluent. Fibrils were dried at 100° C. at 5″ Hg vacuum. Due to transfer losses on filtration, accurate weight gains could not be obtained. Results are listed in Table 1.

TABLE I
Summary of Reactions

| | | | SAMPLE | FIBRIL | | | DRY Wt | SO₃H CONC |
|---|---|---|---|---|---|---|---|---|
| EX. | RUN # | REACT | Wt. g | TYPE | T° C. | TIME | GAIN | meg/g |
| 1A | 118-60A | Vap | 0.20 | CY | 110 | 15 m | 9.3% | 0.50 |
| 1B | 118-61A | Vap | 0.20 | BN | 100 | 30 m | 8.5% | 0.31 |
| 1C | 118-61B | Vap | 0.20 | BN | 65 | 15 m | 4.2% | 0.45 |
| 1D | 118-56A | Liq | 1.2 | CY | 50 | 10 m | | 0.33 |
| 1E | 118-56B | Liq | 1.0 | CY | 25 | 20 m | | 0.40 |

There was no significant difference in sulfonic acid content by reaction in the vapor phase or liquid phase. There was a temperature effect. Higher temperature of reaction (vapor phase) gives higher amounts of sulfones. In 118-61B, the 4.2% wt gain agreed with the sulfonic acid content (theoretical was 0.51 meq/g). Runs 60A and 61A had too high a wt gain to be accounted for solely by sulfonic acid content. It was therefore assumed that appreciable amounts of sulfones were also made.

### 2. Additions to Oxide-Free Fibril Surfaces

Background techniques are described in Urry, G., Elementary Equilibrium Chemistry of Carbon, Wiley, New York 1989.

The surface carbons in fibrils behave like graphite, i.e., they are arranged in hexagonal sheets containing both basal plane and edge carbons. While basal plane carbons are relatively inert to chemical attack, edge carbons are reactive and must contain some heteroatom or group to satisfy carbon valency. Fibrils also have surface defect sites which are basically edge carbons and contain heteroatoms or groups.

The most common heteroatoms attached to surface carbons of fibrils are hydrogen, the predominant gaseous component during manufacture; oxygen, due to its high reactivity and because traces of it are very difficult to avoid; and H₂O, which is always present due to the catalyst. Pyrolysis at ˜1000° C. in a vacuum will deoxygenate the surface in a complex reaction with unknown mechanism, but with known stoichiometry. The products are CO and CO₂, in a 2:1 ratio. The resulting fibril surface contains radicals in a C₁-C₄ alignment which are very reactive to activated olefins. The surface is stable in a vacuum or in the presence of an inert gas, but retains its high reactivity until exposed to a reactive gas. Thus, fibrils can be pyrolized at ˜1000° C. in vacuum or inert atmosphere, cooled under these same conditions and reacted with an appropriate molecule at lower temperature to give a stable functional group. Typical examples are:

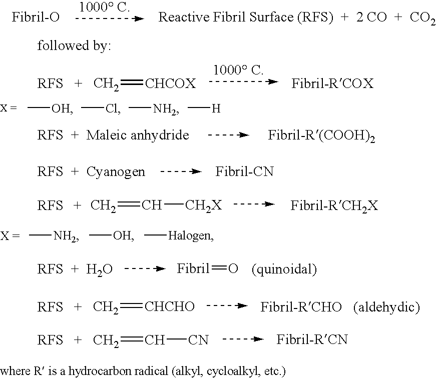

### EXAMPLE 2

### Preparation of Functionalized Fibrils by Reacting Acrylic Acid with Oxide-Free Fibril Surfaces

One gram of BN fibrils in a porcelain boat is placed in a horizontal 1″ quartz tube fitted with a thermocouple and situated in a Lindberg tube furnace. The ends are fitted with a gas inlet/outlets. The tube is purged with dry, deoxygenated argon for 10 minutes, after which the temperature of the furnace is raised to 300° C. and held for 30 minutes. Thereafter, under a continued flow of argon, the temperature is raised in 100° C. increments to 1000° C., and held there for 16 hours. At the end of that time, the tube is cooled to room temperature (RT) under flowing argon. The flow of argon is then shunted to pass through a multi-neck flask containing neat purified acrylic acid at 50° C. and fitted with gas inlet/outlets. The flow of acrylic acid/argon vapors is continued at RT for 6 hours. At the end of that time, residual unreacted acrylic acid is removed, first by purging with argon, then by vacuum drying at 100° C. at <5″ vacuum. The carboxylic acid content is determined by reaction with excess 0.10N NaOH and back-titrating with 0.100N HCl to an endpoint at pH 7.5.

### EXAMPLE 3

### Preparation of Functionalized Fibrils by Reacting Acrylic Acid with Oxide-Free Fibril Surfaces

The procedure is repeated in a similar manner to the above procedure, except that the pyrolysis and cool-down are carried out at 10⁻⁴ Torr vacuum. Purified acrylic acid vapors are diluted with argon as in the previous procedure.

### EXAMPLE 4

### Preparation of Functionalized Fibrils by Reacting Maleic Acid with Oxide-Free Fibril Surfaces

The procedure is repeated as in Ex. 2, except that the reactant at RT is purified maleic anhydride (MAN) which is fed to the reactor by passing argon gas through a molten MAN bath at 80° C.

### EXAMPLE 5

### Preparation of Functionalized Fibrils by Reacting Acryloyl Chloride with Oxide-Free Fibril Surfaces

The procedure is repeated as in Ex. 2, except that the reactant at RT is purified acryloyl chloride, which is fed to the reactor by passing argon over neat acryloyl chloride at 25° C. Acid chloride content is determined by reaction with excess 0.100N NaOH and back-titration with 0.100N HCl.

Pyrolysis of fibrils in vacuum deoxygenates the fibril surface. In a TGA apparatus, pyrolysis at 1000° C. either in vacuum or in a purified Ar flow gives an average wt loss of 3% for 3 samples of BN fibrils. Gas chromatographic analyses detected only CO and CO₂, in ˜2:1 ratio, respectively. The resulting surface is very reactive and activated olefins such as acrylic acid, acryloyl chloride, acrylamide, acrolein, maleic anhydride, allyl amine, allyl alcohol or allyl halides will react even at room temperature to form clean products containing only that functionality bonded to the activated olefin. Thus, surfaces containing only carboxylic acids are available by reaction with acrylic acid or maleic anhydride; surf only acid chloride by reaction with acryloyl chloride; only aldehyde from acrolein; only hydroxyl from allyl alcohol; only amine from allyl amine, and only halide from allyl halide.

### 3. Metallation

Background techniques are given in March, Advanced Organic Chemistry, 3rd ed., p 545.

Aromatic C—H bonds can be metallated with a variety of organometallic reagents to produce carbon-metal bonds (C-M). M is usually Li, Be, Mg, Al, or Tl; however, other metals can also be used. The simplest reaction is by direct displacement of hydrogen in activated aromatics:

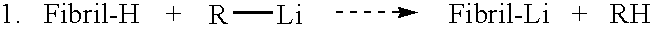

The reaction may require additionally, a strong base, such as potassium t-butoxide or chelating diamines. Aprotic solvents are necessary (paraffins, benzene).

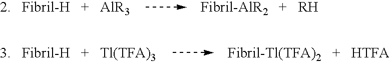

TFA=Trifluoroacetate HTFA=Trifluoroacetic acid

The metallated derivatives are examples of primary singly-functionalized fibrils. However, they can be reacted further to give other primary singly-functionalized fibrils. Some reactions can be carried out sequentially in the same apparatus without isolation of intermediates.

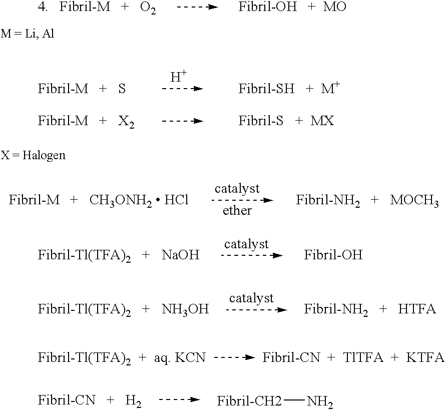

### EXAMPLE 6

### Preparation of Fibril-Li

One gram of CC fibrils is placed in a porcelain boat and inserted into a 1″ quartz tube reactor which is enclosed in a Lindberg tube furnace. The ends of the tube are fitted with gas inlet/outlets. Under continuous flow of H₂, the fibrils are heated to 700° C. for 2 hours to convert any surface oxygenates to C—H bonds. The reactor is then cooled to RT under flowing H₂.

The hydrogenated fibrils are transferred with dry, deoxygenated heptane (with LiAlH₄) to a 1 liter multi-neck round bottom flask equipped with a purified argon purging system to remove all air and maintain an inert atmosphere, a condenser, a magnetic stirrer and rubber septum through which liquids can be added by a syringe. Under an argon atmosphere, a 2% solution containing 5 mmol butyllithium in heptane is added by syringe and the slurry stirred under gentle reflux for 4 hours. At the end of that time, the fibrils are separated by gravity filtration in an argon atmosphere glove box and washed several times on the filter with dry, deoxygenated heptane. Fibrils are transferred to a 50 cc r.b. flask fitted with a stopcock and dried under 10⁻⁴ torr vacuum at 50° C. The lithium concentration is determined by reaction of a sample of fibrils with excess 0.100N HCl in DI water and back-titration with 0.100N NaOH to an endpoint at pH 5.0.

### EXAMPLE 7

### Preparation of Fibril-Tl(TFA)₂

One gram of CC fibrils are hydrogenated as in Ex. 5 and loaded into the multi-neck flask with HTFA which has been degassed by repeated purging with dry argon. A 5% solution of 5 mmol Tl(TFA)₃ in HTFA is added to the flask through the rubber septum and the slurry is stirred at gentle reflux for 6 hours. After reaction, the fibrils are collected and dried as in Ex. 1.

### EXAMPLE 8

### Preparation of Fibril-OH

### (Oxygenated Derivative containing Only OH Functionalization)

One half g of lithiated fibrils prepared in Ex. 6 are transferred with dry, deoxygenated heptane in an argon-atmosphere glove bag to a 50 cc single neck flask fitted with a stopcock and magnetic stirring bar. The flask is removed from the glove bag and stirred on a magnetic stirrer. The stopcock is then opened to the air and the slurry stirred for 24 hours. At the end of that time, the fibrils are separated by filtration and washed with aqueous MeOH, and dried at 50° C. at 5″ vacuum. The concentration of OH groups is determined by reaction with a standardized solution of acetic anhydride in dioxane (0.252 M) at 80° C. to convert the OH groups to acetate esters, in so doing, releasing 1 equivalent of acetic acid/mole of anhydride reacted. The total acid content, free acetic acid and unreacted acetic anhydride, is determined by titration with 0.100N NaOH to an endpoint at pH 7.5.

### EXAMPLE 9

### Preparation of Fibril-NH₂

One gram of thallated fibrils is prepared as in Ex. 7. The fibrils are slurried in dioxane and 0.5 g triphenyl phosphine dissolved in dioxane is added. The slurry is stirred at 50° C. for several minutes, followed by addition at 50° C. of gaseous ammonia for 0.30 min. The fibrils are then separated by filtration, washed in dioxane, then DI water and dried at 80° C. at 5″ vacuum. The amine concentration is determined by reaction with excess acetic anhydride and back-titration of free acetic acid and unreacted anhydride with 0.100N NaOH.

### 4. Derivatized Polynuclear Aromatic. Polyheteronuclear Aromatic and Planar Macrocyclic Compounds

The graphitic surfaces of fibrils allow for physical adsorption of aromatic compounds. The attraction is through van der Waals forces. These forces are considerable between multi-ring heteronuclear aromatic compounds and the basal plane carbons of graphitic surfaces. Desorption may occur under conditions where competitive surface adsorption is possible or where the adsorbate has high solubility.

For example, it has been found that fibrils can be functionalized by the adsorption of phthalocyanine derivatives. These phthalocyanine derivative fibrils can then be used as solid supports for protein immobilization. Different chemical groups can be introduced on the fibril surface simply by choosing different derivatives of phthalocyanine.

The use of phthalocyanine derivative fibrils for protein immobilization has significant advantages over the prior art methods of protein immobilization. In particular, it is simpler than covalent modifications. In addition, the phthalocyanine derivative fibrils have high surface area and are stable in almost any kind of solvent over a wide range of temperature and pH.

### EXAMPLE 10

### Adsorption of Porphyrins and Phthalocyanines onto Fibrils

The preferred compounds for physical adsorption on fibrils are derivatized porphyrins or phthalocyanines which are known to adsorb strongly on graphite or carbon blacks. Several compounds are available, e.g., a tetracarboxylic acid porphyrin, cobalt (II) phthalocyanine or dilithium phthalocyanine. The latter two can be derivatized to a carboxylic acid form.

Dilithium Phthalocyanine

In general, the two Li⁺ ions are displaced from the phthalocyanine (Pc) group by most metal (particularly multi-valent) complexes. Therefore, displacement of the Li⁺ ions with a metal ion bonded with non-labile ligands is a method of putting stable functional groups onto fibril surfaces. Nearly all transition metal complexes will displace Li⁺ from Pc to form a stable, non-labile chelate. The point is then to couple this metal with a suitable ligand.

Cobalt (II) Phthalocyanine

Cobalt (II) complexes are particularly suited for this. Co⁺⁺ ion can be substituted for the two Li⁺ ions to form a very stable chelate. The Co⁺⁺ ion can then be coordinated to a ligand such as nicotinic acid, which contains a pyridine ring with a pendant carboxylic acid group and which is known to bond preferentially to the pyridine group. In the presence of excess nicotinic acid, Co(II)Pc can be electrochemically oxidized to Co(III)Pc, forming a non-labile complex with the pyridine moiety of nicotinic acid. Thus, the free carboxylic acid group of the nicotinic acid ligand is firmly attached to the fibril surface.

Other suitable ligands are the aminopyridines or ethylenediamine (pendant NH₂), mercaptopyridine (SH), or other polyfunctional ligands containing either an amino- or pyridyl-moiety on one end, and any desirable function on the other.

The loading capacity of the porphyrin or phthalocyanines can be determined by decoloration of solutions when they are added incrementally. The deep colors of the solutions (deep pink for the tetracarboxylic acid porphyrin in MeOH, dark blue-green for the CO(II) or the dilithium phthalocyanine in acetone or pyridine) are discharged as the molecules are removed by adsorption onto the black surface of the fibrils.

Loading capacities were estimated by this method and the footprints of the derivatives were calculated from their approximate measurements (−140 sq. Angstroms). For an average surface area for fibrils of 250 m²/g, maximum loading will be −0.3 mmol/g.

The tetracarboxylic acid porphyrin was analyzed by titration. The integrity of the adsorption was tested by color release in aqueous systems at ambient and elevated temperatures.

The fibril slurries were initially mixed (Waring blender) and stirred during loading. Some of the slurries were ultra-sounded after color was no longer discharged, but with no effect.

After loading, Runs 169-11, -12, -14 and -19-1 (see Table II) were washed in the same solvent to remove occluded pigment. All gave a continuous faint tint in the wash effluent, so it was difficult to determine the saturation point precisely. Runs 168-18 and -19-2 used the calculated amounts of pigment for loading and were washed only very lightly after loading.

The tetracarboxylic acid porphyrin (from acetone) and the Co phthalocyanine (from pyridine) were loaded onto fibrils for further characterization (Runs 169-18 and -19-2, respectively).

Analysis of Tetracarboxylic Acid Porphyrin

Addition of excess base (pH 11-12) caused an immediate pink coloration in the titrating slurry. While this did not interfere with the titration, it showed that at high pH, porphyrin desorbed. The carboxylic acid concentration was determined by back titration of excess NaOH using Ph 7.5 as end-point. The titration gave a loading of 1.10 meq/g of acid, equivalent to 0.275 meq/g porphyrin.

Analysis of Cobalt or Dilithium Phthalocyanine

The concentrations of these adsorbates were estimated from decoloration experiments only. The point where the blue-green tint did not fade after 30 min was taken as the saturation-point.

A number of substituted polynuclear aromatic or polyheteronuclear aromatic compounds were adsorbed on fibril surfaces. For adhesion, the number of aromatic rings should be greater than two per rings/pendant functional group. Thus, substituted anthracenes, phenanthrenes, etc., containing three fused rings, or polyfunctional derivatives containing four or more fused rings can be used in place of the porphyrin or phthalocayanine derivatives. Likewise, substituted aromatic heterocycles such as the quinolines, or multiply substituted heteroaromatics containing four or more rings can be used.

Table II summarizes the results of the loading experiments for the three porphyrin/phthalocyanine derivatives.

TABLE II
Summary of Adsorption Runs

| | | | Wgt. | | Loading | | meq/g |
|---|---|---|---|---|---|---|---|
| EX. | RUN # | Adsorbate | Fib, g | Solv. | g/g | Form | Titration |
| 10A | 169-11 | TCAPorph | 19.6 mg | Acet | 0.18 | Acid | na |
| | | | | | g/g | | |
| 10B | 169-12 | TCAPorph | 33.3 mg | H₂O | 0.11 | Na | na |
| | | | | | | Salt | |
| 10C | 169-14 | DiLiPhth | 119.0 mg | Acet | 0.170 | Li | na |
| 10D | 169- | CoPhth | 250.0 mg | Pyr | 0.187 | Co | 0.335(cal) |
| | 19-1 | | | | | | |
| 10E | 169-18 | TCAPorph | 1.00 g | Acet | 0.205 | Acid | 1.10(T) |
| 10F | 169- | CoPhth | 1.40 g | Pyr | 0.172 | Co | 0.303(cal) |
| | 19-2 | | | | | | |

TCAPorph = Tetracarboxylic Acid Porphyrin
(cal) = calculated
DiLiPhth = Dilithium Phthalocyanine
(T) = Titration
CoPhth = Cobalt (II) Phthalocyanine

The following Examples 11 and 12 illustrate methods for the adsorption of two different phthalocyanine derivatives on carbon nanotubes.

### EXAMPLE 11

### Fibrils Punctionalized by Adsorption of Nickel (II) Phthalocyaninetetrasulfonic Acid

Two milligrams of Nickel (II) phthalocyanine-tetrasulfonic acid (tetrasodium salt) was mixed with 4.2 milligrams of plain fibrils in one milliliter of dH₂O. The mixture was sonicated for 50 minutes and rotated at room temperature overnight.

The fibrils were washed with 3×1 ml of dH₂O, 3×1 ml of MeOH, and 3×1 ml of CH₂Cl₂ and dried under vacuum.

Thermolysin was immobilized on these phthalocyanine derivative fibrils by adsorption. 0.5 mg of fibrils were suspended in 250 μl of dH₂O and sonicated for 20 minutes. The supernatant was discarded and the fibrils were suspended in 250 μl of 0.05 M Tris (pH=8.0) and mixed with 250 μl of 0.6 mM thermolysin solution made in the same buffer. The mixture was rotated at room temperature for 2 hours and stored at 4° C. overnight. The fibrils were then washed three times with 1 ml of 25 mM Tris (pH=8) and suspended in 250 μl of buffer containing 40 mM Tris and 10 mM CaCl₂ at pH 7.5.

The amount of thermolysin on these fibrils was determined by measuring the enzyme activity of the fibrils. Thermolysin can react with substrate FAGLA (N-(3-[2-furyl]acryloyl)-gly-leuamide) and produce a compound that causes absorbance decrease at 345 nm with extinction coefficient of −310 M¹ cm⁻¹. The assay buffer condition for this reaction was 40 mM Tris, 10 mM CaCl₂ and 1.75 M NaCl at pH 7.5. The reaction was performed in 1 ml cuvette by mixing 5 μl of FAGLA stock solution (25.5 mM in 30% DMF in dH₂O) and 10 μg of thermolysin fibrils in 1 ml of assay buffer. The absorbance decrease at 345 nm was monitored by time scan over 10 minutes. The enzyme activity (μM/min) was then calculated from the initial slope using the extinction coefficient −310 M⁻¹ cm⁻¹. The amount of active thermolysin per gram of fibril was 0.61 μ moles.

### EXAMPLE 12

### Fibrils Functionalized by Adsorption of 1,4,8,11,15,18,22,25-Octabutoxy-29H,31H-phthalocyanine

Three milligrams of 1,4,8,11,15,22,25-octabutoxy-29H,31H-phthalocyanine and 5.3 mg of plain fibrils were mixed in 1 ml of CHCl₃. The mixture was sonicated for 50 minutes and rotated at room temperature overnight.

The fibrils were washed with 3×1 ml of CH₂Cl₂ and dried under vacuum.

Thermolysin was immobilized on these phthalocyanine derivative fibrils by adsorption according to the method of Example 34. The amount of active thermolysin per gram of fibrils was 0.70 μmoles.

### EXAMPLE 13

### Aspartame Precursor Synthesis Using Phthalocyanine Derivative Fibrils with Thermolysin Immobilized Thereon

Phthalocyanine derivative fibrils on which thermolysin has been immobilized can be used to catalyze the synthesis of a precursor of the artificial sweetener aspartame. The reaction is carried out by mixing 80 mM L-Z-Asp and 220 mM L-PheOMe in ethyl acetate with 10 μM fibril immobilized thermolysin. The product Z-Asp-PheOMe is monitored by HPLC to determine the yield.

### 5. Chlorate or Nitric Acid Oxidation

Literature on the oxidation of graphite by strong oxidants such as potassium chlorate in conc. sulfuric acid or nitric acid, includes R. N. Smith, Quarterly Review 13, 287 (1959); M. J. D. Low, Chem. Rev. 60, 267 (1960)). Generally, edge carbons (including defect sites) are attacked to give mixtures of carboxylic acids, phenols and other oxygenated groups. The mechanism is complex involving radical reactions.

### EXAMPLE 14

### Preparation of Carboxylic Acid-Functionalized Fibrils Using Chlorate

The sample of CC fibrils was slurried in conc. H₂SO₄ by mixing with a spatula and then transferred to a reactor flask fitted with gas inlet/outlets and an overhead stirrer. With stirring and under a slow flow of argon, the charge of NaClO₃ was added in portions at RT over the duration of the run. Chlorine vapors were generated during the entire course of the run and were swept out of the reactor into a aqueous NaOH trap. At the end of the run, the fibril slurry was poured over cracked ice and vacuum filtered. The filter cake was then transferred to a Soxhlet thimble and washed in a Soxhlet extractor with DI water, exchanging fresh water every several hours. Washing was continued until a sample of fibrils, when added to fresh DI water, did not change the pH of the water. The fibrils were then separated by filtration and dried at 100° C. at 5″ vacuum overnight.

The carboxylic acid content was determined by reacting a sample with excess 0.100N NaOH and back-titrating with 0.100ⁿ HCl to an endpoint at pH 7.5. The results are listed in the Table.

TABLE III
Summary of Direct Oxidation Runs

| | | Components, g | | | | Rec | | |
|---|---|---|---|---|---|---|---|---|
| Acid, | | | | cc | Time | Wash | | meq/ |
| Ex. | RUN # | Fibrils | NaClO₃ | H₂SO₄ | hours | Ph | Wgt | g |
| 11A | 168-30 | 10.0 | 8.68 | 450 | 24 | 5.7 | 10.0 | 0.78 |
| 11B | 168-36 | 12.0 | 13.9 | 600 | 24 | 5.9 | 13.7 | 0.75 |

### EXAMPLE 15

### Preparation of Carboxylic Acid-Functionalized Fibrils Using Nitric Acid

A weighed sample of fibrils was slurried with nitric acid of the appropriate strength in a bound bottom multi-neck indented reactor flask equipped with an overhead stirrer and a water condenser. With constant stirring, the temperature was adjusted and the reaction carried out for the specified time. Brown fumes were liberated shortly after the temperature exceeded 70° C., regardless of acid strength. After the reaction, the slurry was poured onto cracked ice and diluted with DI water. The slurry was filtered and excess acid removed by washing in a Soxhlet extractor, replacing the reservoir with fresh DI water every several hours, until a slurried sample gave no change in Ph from DI water. The fibrils were dried at 100° C. at 5″ vacuum overnight. A weighed portion of fibrils was reacted with standard 0.100 N NaOH and the carboxylic acid content determined by back-titration with 0.100 N HCl. Surface oxygen content was determined by XPS. Dispersibility in water was tested at 0.1 wt % by mixing in a Waring Blender at high for 2 min. Results are summarized in Table 4.

TABLE IV
Summary of Direct Oxidation Runs

| | COMPONENTS | | | | | | | | | |
|---|---|---|---|---|---|---|---|---|---|---|
| | Gms. | cc | Acid | Temp. | | Wgt. | COOH | | | Disp |
| Ex. | Fibrils | Acid | Conc. | ° C. | Time | Loss | meq/g | ESCA, C | at % O | H₂O |
| 12A | 1(BN) | 300 | 70% | RT | 24 hr | 0 | <0.1 | 98 | 2 | P |

| | | | | | | | | | |
|---|---|---|---|---|---|---|---|---|---|
| 12B | 1(BN) | 300 | 15 | rflx | 48 | <5% | <0.1 | not analyzed | P |
| 12C | 20(BN) | 1.0 1 | 70 | rflx | 7 | 25% | 0.8 | not analyzed | G |
| 12D | 48(BN) | 1.0 1 | 70 | rflx | 7 | 20% | 0.9 | not analyzed | G |

P = Poor;
G = Good

### 6. Amino Functionalization of Fibrils

Amino groups can be introduced directly onto graphitic fibrils by treating the fibrils with nitric acid and sulfuric acid to get nitrated fibrils, then reducing the nitrated form with a reducing agent such as sodium dithionite to get amino-functionalized fibrils according to the following formula:

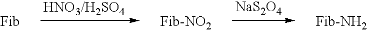

The resulting fibrils have many utilities, including the immobilization of proteins (e.g., enzymes and antibodies), and affinity and ion exchange chromatography.

### EXAMPLE 16

### Preparation of Amino-Functionalized Fibrils Using Nitric Acid

To a cooled suspension (0° C.) of fibrils (70 mg) in water (1.6 ml) and acetic acid (0.8 ml) was added nitric acid (0.4 ml) in a dropwise manner. The reaction mixture was stirred for 15 minutes at 0° C. and stirred for further 1 hour at room temperature. A mixture of sulfuric acid (0.4 ml) and hydrochloric acid (0.4 ml) was added slowly and stirred for 1 hour at room temperature. The reaction was stopped and centrifuged. The aqueous layer was removed and the fibrils washed with water (×5). The residue was treated with 10% sodium hydroxide (×3), and washed with water (×5) to furnish nitrated fibrils.

To a suspension of nitrated fibrils in water (3 ml) and ammonium hydroxide (2 ml) was added sodium dithionite (200 mg) in three portions at 0° C. The reaction mixture was stirred for 5 minutes at room temperature and refluxed for 1 hour at 100° C. The reaction was stopped, cooled to 0° C. and the pH adjusted with acetic acid (pH 4). After standing overnight at room temperature, the suspension was filtered, washed with water (×10), methanol (×5) and dried in vaccuo to give amino fibrils.

To test the amino functionalized fibrils, the fibrils were coupled with horseradish peroxidaese. The HRP-coupled amino fibrils were then extensively dialyzed. Following dialysis, the fibrils were washed 15 times over the following week. The enzyme-modified fibrils were assayed as follows:

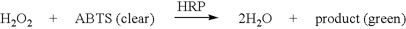

The results indicated that HRP coupled with Fib-NH₂ showed good enzyme activity which was retained over a period of one week.

### 7. Attachment of Terminal Alcohols Using a Free Radical Initiator

The high degree of stability of carbon nanotubes, while allowing them to be used in harsh environments, makes them difficult to activate for further modification. Previous methods have involved the use of harsh oxidants and acids. It has now been surprisingly found that terminal alcohols can be attached to carbon nanotubes using a free radical initiator such as benzoyl peroxide (BPO). Carbon nanotubes are added to an alcohol having the formula RCH₂OH, wherein R is hydrogen, alkyl, aryl, cycloalkyl, aralkyl, cycloaryl, or poly(alkylether) along with a free radical initiator and heated to from about 60° C. to about 90° C. Preferred alcohols include ethanol and methanol. When sufficient time has elapsed for all of the free radical initiator to decompose, the reaction mixture is filtered and the carbon nanotube material is washed and dried, yielding modified nanotubes of the formula Nanotube-CH(R)OH. This method can also be used to couple bifunctional alcohols. This allows one end to be linked to the carbon nanotube and the other to be used for the indirect linkage of another material to the surface.

### EXAMPLE 17

### Preparation of Alcohol Functionalized Nanotubes Using Benzoyl Peroxide

0.277 grams of carbon nanotubes were dispersed in MeOH using a probe sonicator. 0.126 grams of BPO were added at RT and the temperature was increased to 60° C. and an additional 0.128 grams of BPO were added. After an additional 45 minutes at 60° C., a final BPO charge of 0.129 grams was added and the mixture was kept at 60° C. for an additional 30 minutes. The product was filtered onto a membrane and washed several times with MeOH and EtOH and dried in an oven at 90° C. The yield was 0.285 grams. ESCA analysis showed an oxygen content of 2.5 atomic percent compared with 0.74% for a control sample refluxed in MeOH without BPO.

### EXAMPLE 18

### Modification of Carbon Nanotubes with Poly(ethylene Glycol) Using Benzoyl Peroxide

0.1 grams of carbon nanotubes, 0.5 grams BPO and 10 grams poly(ethyleneglycol), avg. mol. wt. 1000 (PEG-1000) were mixed together at room temperature. The mixture was heated to 90° C. to melt the PEG and the mixture was left to react at 90° C. overnight. The entire mixture was then filtered and washed to remove the excess PEG and was then dried. The resultant material can be used either as is, or it can be further modified by attaching materials of interest to the free end of the PEG.

### EXAMPLE 19

### Use of Carbon Nanotubes Modified With PEG to Reduce Nonspecific Binding

Non-specific binding to high surface area carbon material is ubiquitous. It has been found that attaching hydrophilic oligomers such as PEG to carbon nanotubes can reduce non-specific binding. Further, it has been found that by attaching one end of chain-like molecules such as PEG to the surface of the nanotubes the free end can contain a functional group that can be used for attachment of other materials of interest while still retaining the properties of the PEG (or other material) layer to reduce non-specific binding.

Reduction of Non-specific Binding of Bovine Serum Albumen with PEG-modified Fibrils

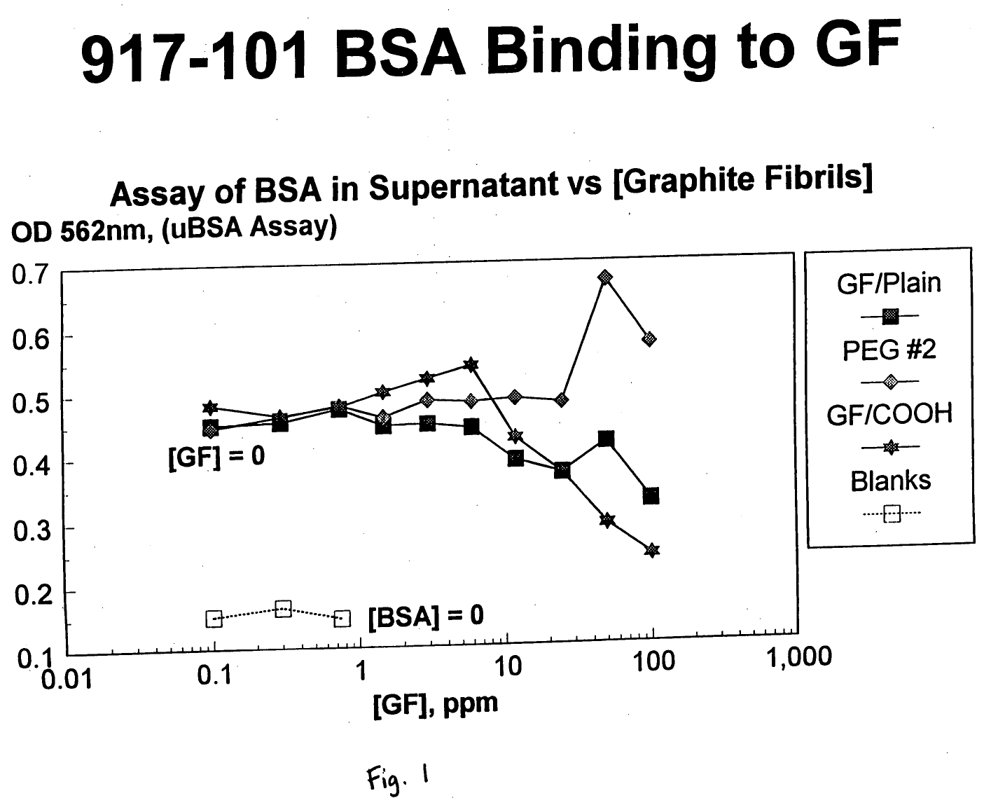

Stock dispersions of unmodified fibrils, chlorate oxidized fibrils and PEG modified fibrils at 0.1 mg/ml in 50 mm potassium phosphate buffer at pH 7.0 were prepared by dispersing 1.0 mg of each in 10 mls of buffer with sonication. 2 mls of 2-fold serial dilutions of each were placed in each of 9 polypropylene tubes. 100 μl of a 0.2 mg/ml solution of bovine serum albumin (BSA) in the same buffer was added to each tube and to three buffer blanks. Three buffer tubes without protein were also prepared. All tubes were mixed on a vortex mixer and allowed to incubate for 30 minutes with 30 seconds of vortexing every 10 minutes. All tubes were centrifuged to separate the fibrils and 1 ml aliquots of the supernatant were transferred to new tubes and analyzed for total protein content using a Micro BCA protein assay (Pierce). The level of protein remaining in the supernatant was an indirect measure of the amount that had been non-specifically bound to the fibrils. All the BSA remained in the supernatant for the PEG modified fibrils while there was nearly complete binding to the unmodified or chlorate oxidized fibrils (see FIG. 1).

Comparison of Reduction of Non-Specific Binding by PEG-Modified Fibrils Prepared Using Benzoyl Peroxide and by NHS Ester Coupling

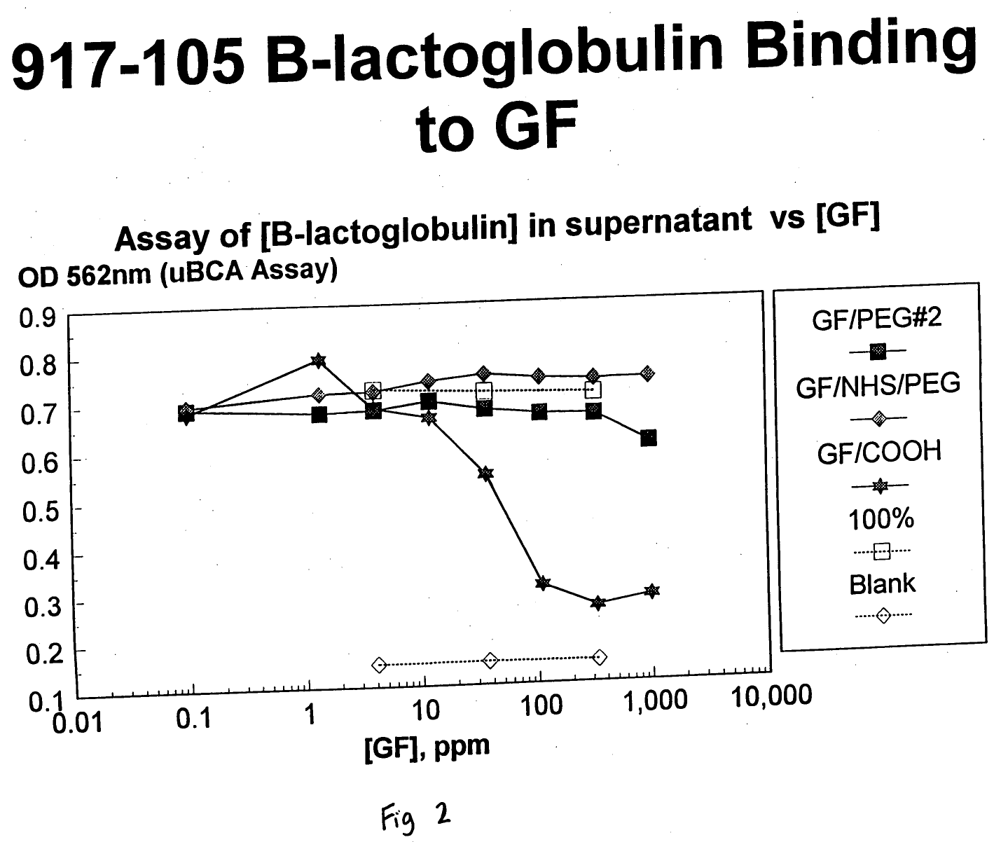

Stock dispersions of chlorate oxidized fibrils, fibrils modified with PEG using benzoyl peroxide and chlorate oxidized fibrils modified with PEG by NHS ester coupling were prepared at 1.0 mg/ml in 50 mM potassium phosphate buffer, pH 7.0 with sonication. 2 mls of 3-fold serial dilutions of each were placed in each of 7 polypropylene tubes. 100 μl of a 0.2 mg/ml solution of β-lactoglobulin (βLG) in the same buffer was added to each tube and to 3 buffer blanks. Three buffer tubes without protein were also prepared. All tubes were mixed on a vortex mixer and allowed to incubate for 60 minutes with 30 seconds of vortexing every 10 minutes. All tubes were centrifuged to separate the fibrils and 1 ml aliquots of the supernatant were transferred to new tubes and analyzed for total protein content using a Micro BCA protein assay (Pierce). The level of protein remaining in the supernatant was an indirect measure of the amount that had been non-specifically bound to the protein (see FIG. 2). For each of the tubes the βLG remained in the supernatant for the fibrils modified with PEG via the NHS ester route signifying no non-specific binding. The fibrils modified with PEG via the BPO route exhibited only slight (approx. 10%) binding of the βLG at the highest fibril level of 1.0 mg/ml and no significant binding at lower levels. In contrast, there was nearly complete binding to the chlorate oxidized fibrils at fibril levels of 0.1 mg/ml and above and substantial binding down to 0.01 mg/ml of these fibrils.

### 8. Secondary Derivatives of Functionalized Nanotubes

Carboxylic Acid-functionalized Nanotubes

The number of secondary derivatives which can be prepared from just carboxylic acid is essentially limitless. Alcohols or amines are easily linked to acid to give stable esters or amides. If the alcohol or amine is part of a di- or bifunctional poly-functional molecule, then linkage through the O— or NH— leaves the other functionalities as pendant groups. Typical examples of secondary reagents are:

| | PENDANT | |
|---|---|---|
| GENERAL FORMULA | GROUP | EXAMPLES |
| HO—R, R = alkyl, aralkyl, | R— | Methanol, phenol, tri- |
| aryl, fluoroethanol, | | fluorocarbon, OH-terminated |
| polymer, SiR'₃ | | Polyester, silanols |
| H₂N—R R = same as | R— | Amines, anilines, |
| above | | fluorinated amines, |
| | | silylamines, amine |
| | | terminated polyamides, |
| | | proteins |
| Cl—SiR₃ | SiR₃— | Chlorosilanes |
| HO—R—OH, R = alkyl, | HO— | Ethyleneglycol, PEG, Penta- |
| aralkyl, CH₂O— | | erythritol, bis-Phenol A |
| H₂N—R—NH₂, R = alkyl, | H₂N— | Ethylenediamine, |
| aralkyl | | polyethyleneamines |
| X—R—Y, R = alkyl, etc; | Y— | Polyamine amides, |
| X═OH or NH₂; Y═SH, | | Mercaptoethanol |
| CN, C═O, CHO, alkene, | | |
| alkyne, aromatic, | | |
| heterocycles | | |

The reactions can be carried out using any of the methods developed for esterifying or aminating carboxylic acids with alcohols or amines. Of these, the methods of H. A. Staab, Angew. Chem. Internat. Edit., (1), 351 (1962) using N,N′-carbonyl diimidazole (CDI) as the acylating agent for esters or amides, and of G. W. Anderson, et al., J. Amer. Chem. Soc. 86, 1839 (1964), using N-hydroxysuccinimide (NHS) to activate carboxylic acids for amidation were used.

### EXAMPLE 20

### Preparation of Secondary Derivatives of Functionalized Fibrils

N,N′-Carbonyl Diimidazole

Clean, dry, aprotic solvents (e.g., toluene or dioxane) are required for this procedure. Stoichiometric amounts of reagents are sufficient. For esters, the carboxylic acid compound is reacted in an inert atmosphere (argon) in toluene with a stoichiometric amount of CDI dissolved in toluene at R.T. for 2 hours. During this time, CO₂ is evolved. After two hours, the alcohol is added along with catalytic amounts of Na ethoxide and the reaction continued at 80° C. for 4 hr. For normal alcohols, the yields are quantitative. The reactions are:

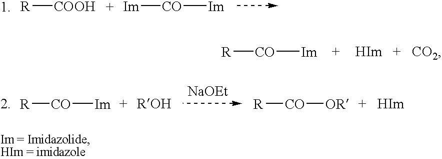

Amidation of amines occurs uncatalyzed at RT. The first step in the procedure is the same. After evolution of CO₂, a stoichiometric amount of amine is added at RT and reacted for 1-2 hours. The reaction is quantitative. The reaction is:

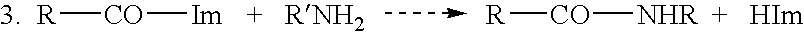

Silylation

Trialkylsilylchlorides or trialkylsilanols react immediately with acidic H according to:

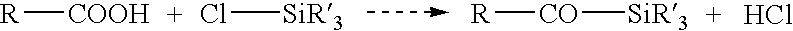

Small amounts of Diaza-1,1,1-bicyclooctane (DABCO) are used as catalysts. Suitable solvents are dioxane and toluene.

Sulfonic Acid-Functionalized Fibrils

Aryl sulfonic acids, as prepared in Example 1, can be further reacted to yield secondary derivatives. Sulfonic acids can be reduced to mercaptans by LiAlH₄ or the combination of triphenyl phosphine and iodine (March, J. P., p. 1107). They can also be converted to sulfonate esters by reaction with dialkyl ethers, i.e.,

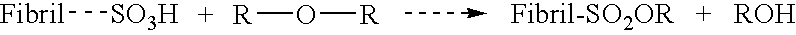

N-Hydroxysuccinimide

Activation of carboxylic acids for amidation with primary amines occurs through the N-hydroxysuccinamyl ester; carbodiimide is used to tie up the water released as a substituted urea. The NHS ester is then converted at RT to the amide by reaction with primary amine. The reactions are:

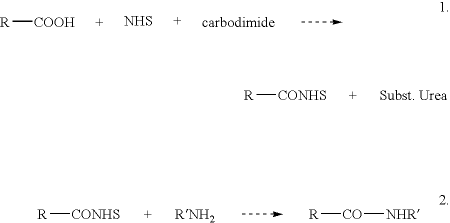

This method is particularly useful for the covalent attachment of protein to graphitic fibrils via the free NH₂ on the protein's side chain. Examples of proteins which can be immobilized on fibrils by this method include trypsin, streptavidin and avidin. The streptavidin (or avidin) fibrils provide a solid carrier for any biotinylated substance

### EXAMPLE 21

### Covalent Attachment of Proteins to Fibrils via NHS Ester

To demonstrate that protein can be covalently linked to fibrils via NHS ester, streptavidin, avidin and trypsin were attached to fibrils as follows.

0.5 mg of NHS-ester fibrils were washed with 5 mM sodium phosphate buffer (pH 7.1) and the supernatant was discarded. 200 μl streptavidin solution (1.5 mg in the same buffer) was added to the fibrils and the mixture was rotated at room temperature for 5.5 hours. The fibrils were then washed with 1 ml of following buffers in sequence: 5 mM sodium phosphate (pH 7.1), PBS (0.1 M sodium phosphate, 0.15 M NaCl, pH 7.4), ORIGEN™ assay buffer (IGEN, Inc., Gaithersburg, Md.) and PBS. The streptavidin fibrils were stored in PBS buffer for further use.

2.25 mg NHS-ester fibrils were sonicated in 500 μl of 5 mM sodium phosphate buffer (pH 7.1) for 40 minutes and the supernatant was discarded. The fibrils were suspended in 500 μl of 5 mM sodium phosphate buffer (pH 7.1) and 300 μl of avidin solution made in the same buffer containing 2 mg avidin (Sigma, A-9390) was added The mixture were rotated at room temperature for two hours, stored at 4° C. overnight and rotated at room temperature for another hour. The fibrils were washed with 1 ml of 5 mM sodium phosphate buffer (pH 7.1) four times and PBS buffer twice. The avidin fibrils were suspended in 200 μl PBS buffer for storage.

Trypsin fibrils were prepared by mixing 1.1 mg NHS-ester fibrils (treated as in avidin fibrils) and 200 μl of 1.06 mM trypsin solution made in 5 mM sodium phosphate buffer (−pH 7.1) and rotating at room temperature for 6.5 hours. The trypsin fibrils were then washed by 1 ml of 5 mM sodium phosphate buffer (pH 7.1) three times and suspended in 400 μl of the same buffer for storage.

### EXAMPLE 22

### Measurement of Enzyme Activity of Trypsin on Fibrils

Trypsin can react with substrate L-BAPNA (Na-benzoyl-L-arginine p-nitroanilide) and release a colored compound that absorbs light at 410 nm. The assay buffer for this reaction was 0.05 M Tris, 0.02 M CaCl₂, pH 8.2. The reaction was performed in 1 ml cuvette by mixing 5 μl of L-BAPNA stock solution (50 mM in 37% DMSO in H₂O) and 10-25 μg of trypsin fibrils in a 1 ml of assay buffer. The absorbance increase at 410 nm was monitored over 10 minutes. The enzyme activity (μM/min) was then calculated from the initial slope.

For covalently bound trypsin fibrils, the activity was 5.24 μM/min per 13 μg fibrils. This result can be converted to the amount of active trypsin on fibrils by dividing the activity of a known concentration of trypsin solution, which was measured to be 46 μM/min per 1 μM trypsin under the same assay conditions. Therefore the amount of active trypsin per gram of fibrils was 8.3 Moles (or 195 mg).

### EXAMPLE 23

### Carbon Nanotubes with Surface Thiols

0.112 gms of amino carbon nanotubes (CN) prepared by modification with ethylenediamine as described in Example 27 (below) were suspended in 20 mls of pH 8.0 0.05 M potassium phosphate buffer containing 50 mM EDTA. The suspension was sonicated with a Branson 450 Watt probe sonicator for 5 minutes to disperse the CN. The resulting suspension was quite thick. Argon was bubbled though the suspension for 30 minutes with stirring. 50 mgs of 2-iminothiolane•HCl was added and the mixture was allowed to react for 70 minutes with continued stirring under argon. The resulting material was filtered onto a polycarbonate membrane filter, washed 2× with buffer, 1× with DI water and 2× with absolute EtOH, all under an argon blanket. The thiol modified CN were placed in a vacuum desiccator and pumped on overnight. Final weight=0.118 gms, 55% conversion, based on weight gain.

A 10 mg sample of thiolated nanotubes was suspended in 10 mls. of DI water with sonication and filtered onto 0.45 μm nylon membrane to form a felt-like mat. The mat section was stored in a vacuum desiccator prior to analysis by ESCA which showed 0.46% sulfur and 1.69% nitrogen, confirming successful conversion to thiol-modified CN.

### EXAMPLE 24

### Attachment of Thiol-Modified Carbon Nanotubes to Gold Surfaces

Gold foil (Alfa/Aesar), 2 cm×0.8 cm, was cleaned with a solution of 1 part 30% H₂O₂ and 3 parts concentrated H₂SO₄ for 10 minutes and rinsed with DI water. The foil piece was connected to an Au wire lead and cycled electrochemically between −0.35 V vs. Ag/AgCl and 1.45 V vs. Ag/AgCl in 1 M H₂SO₄ at 50 mv/sec until the cyclic voltammograms were unchanged, approx. 10 minutes. It was then rinsed with DI water and dried. The large piece was cut into four strips 0.5 cm×0.8 cm.

10 mls of absolute EtOH, deoxygenated by argon purging for 30 min., was placed in each of two glass vials. In one vial was suspended 16 mgs of thiol-modified CN(CN/SH) and 2 Au pieces and in the other vial 1 piece of Au and 10 mgs of the ethylene diamine modified CN used to make the thiol derivative. All manipulations were carried out in an Ar filled glove bag. The vials were sealed under Ar and placed in a chilled ultrasonic bath for 1 hour. The sealed vials were left at RT for 72 hours. The Au samples were removed from the vials, rinsed 3× with EtOH, air dried and placed in protective vials.

The Au foil samples exposed to the CN/ethylenediamine and CN/SH were examined by scanning electron microscopy (SEM) to detect the presence or absence of CN on the surface. Examination at 40,000× revealed the presence of CN distributed over the surface exposed to CN/SH but no CN were observed on the Au foil sample exposed to CN/ethylenediamine.

### EXAMPLE 25

### Preparation of Maleimide Fibrils from Amino Fibrils

Amino fibrils were prepared according to Example 13. The amino fibrils (62.2 mg) were then sonicated in sodium phosphate buffer (5 ml, 5 mM at pH 7.2). Sulfosuccinmidyl-4-(N-maleimidomethyl)cyclohexane-1-carboxylate (SMCC; 28.8 mg, 0.66 mmols; Pierce, Cat. No.22360) was added to the fibril suspension. The reaction mixture was stirred overnight at room temperature. The fibrils were washed with water and methanol, and the product fibrils were dried under vacuum. Antibody immobilization on the product confirmed the presence of maleimide fibrils. Other maleimides with different linkers (e.g., sulfo-SMCC, succinimidyl 4-[p-maleimidophenyl]butyrate [SMPB], sulfo-SMPB, m-maleimidobenzyl-N-hydroxysuccinimide ester (MBS), sulfo-MBS etc.) fibrils can be made through the same method.

The resulting maleimide fibrils can be used as a solid support for the covalent immobilization of proteins, e.g. antibodies and enzymes. Antibodies were covalently immobilized on malemide activated fibrils. The capacity of antibody was 1.84 milligrams per gram of fibrils when amino fibrils obtained from nitration/reduction method (Example 13) were used and 0.875 milligrams per gram of fibrils when amino fibrils derivatized from carboxyl fibrils were used.

### EXAMPLE 26

### Preparation of Ester/Alcohol Derivatives from Carboxylic Acid-Functionalized Fibrils

The carboxylic acid functionalized fibrils were prepared as in Example 14. The carboxylic acid content was 0.75 meq/g. Fibrils were reacted with a stoichiometric amount of CDI in an inert atmosphere with toluene as solvent at R.T. until CO₂ evolution ceased. Thereafter, the slurry was reacted at 80° C. with a 10-fold molar excess of polyethyleneglycol (MW 600) and a small amount of NaOEt as catalyst. After two hours reaction, the fibrils were separated by filtration, washed with toluene and dried at 100° C.

### EXAMPLE 27

### Preparation of Amide/Amine Derivatives from Carboxylic Acid-Functionalized Fibrils (177-041-1)

0.242 g of chlorate-oxidized fibrils (0.62 meq/g) was suspended in 20 ml anhydrous dioxane with stirring in a 100 ml RB flask fitted with a serum stopper. A 20-fold molar excess of N-hydroxysuccinimide (0.299 g) was added and allowed to dissolve. This was followed by addition of 20-fold molar excess of 1-ethyl-3-(3-dimethylaminopropyl)carbodiimide (EDAC) (0.510 g), and stirring was continued for 2 hr at RT. At the end of this period stirring was stopped, and the supernatant aspirated and the solids were washed with anhydrous dioxane and MeOH and filtered on a 0.45 micron polysulfone membrane. The solids were washed with additional MeOH on the filter membrane and vacuum-dried until no further weight reduction was observed. Yield of NHS-activated oxidized fibrils was 100% based on the 6% weight gain observed.

100 μl ethylenediamine (en) was added to 10 ml 0.2 M NaHCO₃ buffer. An equivalent volume of acetic acid (HOAc) was added to maintain the pH near 8. NHS-activated oxidized fibrils (0.310 g) was added with vigorous stirring and reacted for 1 hr. An additional 300 μl of en and 300 μl HOAc was added for an additional 10 min. The solution was filtered on 0.45 micron polysulfone membrane and washed successively with NaHCO₃ buffer, 1% HCl, DI water and EtOH. The solids were dried under vacuo overnight. The HCl salt was converted back to the free amine by reaction with NaOH (177-046-1) for further analysis and reactions.

ESCA was carried out to quantify the amount of N present on the aminated fibrils (GF/NH₂). ESCA analysis of 177-046-1 showed 0.90 at % N (177-059). To further assess how much of this N is present as both accessible and reactive amine groups, a derivative was made by the gas phase reaction with pentafluorobenzaldehyde to produce the corresponding Schiff Base linkages with available primary amine groups. ESCA analysis still showed the 0.91 at % N, as expected, and 1.68 at % F. This translates into a 0.34 at % of N present as reactive primary amine on the aminated fibrils (5 F per pentafluorobenzaldehyde molecule). A level of 0.45 at % N would be expected assuming complete reaction with the free ends of each N. The observed level indicates a very high yield from the reaction of N with NHS-activated fibril and confirms the reactivity of the available free amine groups.

At the level of 0.34 at % N present as free amine calculated from the ESCA data, there would be almost complete coverage of the fibrils by the free amine groups allowing coupling of other materials.

Carboxyl fibrils were also converted to amino fibrils using mono-protected 1,6-diaminohexane (a six-carbon linker), rather than ethylenediamine (a two-carbon linker).

### EXAMPLE 28

### Preparation of Amine Derivatives from Carboxylic Acid Functionalized Fibrils

Carboxyl groups on fibrils can be modified by reacting the carboxyl groups with one amino group of a compound having two or more amino groups (at least one of which is unprotected by groups such as t-Boc or CBZ). The fibrils so generated are amide derivatives in which the amide carbonyl is derived from the fibril carboxyl group and the amide nitrogen is substituted with a group (such as an alkyl group) containing one or more primary amines. The amino groups are then available for use or further modification.

One gram of carbon fibrils was placed in a dry scintered glass filter tunnel, the outlet of which was tightly stoppered with a rubber serum septum, and anhydrous dichloromethane was added to cover. N-Methylmorpholine (758 μL, 7 mmol) was added, the suspension was mixed with the aid of a spatula. Then isobutyl chloroformate (915 μL, 7 mmol) was added, and the suspension mixed periodically for one hour. The mixture was protected from atmospheric moisture by a cover of Parafilm as much as was practical.

Meanwhile, N-boc-1,6-diaminohexane hydrochloride (1.94 g, 7.7 mmol) was partitioned between dichloromethane (10 mL) and 1 M NaOH (10 mL). The lower, organic phase was dried over anhydrous potassium carbonate and filtered through a disposable Pasteur pipette containing a cotton plug, and N-methylmorpholine (758 μL, 7 mmol) was added.

The serum septum was removed from the filter funnel, the reagents were removed from the fibrils by vacuum filtration, and the fibrils were washed with anhydrous dichloromethane. The serum septum was replaced, and the mixture of N-methylmorpholine and monoprotected diaminohexane was added to the fibrils. The mixture was stirred periodically for one hour. Then, the reagents were removed by filtration, and the fibrils were washed successively with dichloromethane, methanol, water, methanol, and dichloromethane.

A 50% mixture of trifluoric acid and dichloromethane was added to the fibrils and the mixture stirred periodically for 20 minutes. The solvents were removed by filtration, and the fibrils were washed successively with dichloromethane, methanol, water, 0.1 M NaOH, and water.

To demonstrate the efficacy of the method, a small sample of amino fibrils were reacted with “activated” horseradish peroxidase (HRP; 5 mg, Pierce) which was modified to specifically react with amino groups. The fibrils were washed repeatedly for several days (by suspension, rotation, and centrifugation in an Eppendorf tube) while kept cold. After approximately two weeks of washing, the enzyme was assayed with H₂O₂/ABTS in glycine buffer, pH 4.4. A green color appeared in the solution within 10 minutes indicating the presence of enzyme. Control fibrils (COOH fibrils treated with activated HRP and washed for the same period of time) showed little if any catalytic activity.

### EXAMPLE 29

### Preparation of Silyl Derivative from Carboxylic Acid-Functionalized Fibrils

Acid functionalized fibrils prepared as in Example 14 were slurried in dioxane in an inert atmosphere. With stirring, a stoichiometric amount of chlorotriethyl silane was added and reacted for 0.5 hr, after which several drops of a 5% solution of DABCO in dioxane was added. The system was reacted for an additional hour, after which the fibrils were collected by filtration and washed in dioxane. The fibrils were dried at 100° C. in 5″ vacuum overnight.

Table 5 summarizes the secondary derivative preparations. The products were analyzed by ESCA for C, O, N, Si and F surface contents.

TABLE V
Summary of Secondary Derivative Preparations

| | | ESCA ANALYSIS, | | | | | |
|---|---|---|---|---|---|---|---|
| | | ATOM % | | | | | |
| REACTANT | PENDANT GROUP | S | C | N | O | Si | F |
| As Grown | — | — | 98.5 | — | 1.5 | — | — |
| Chlorate | —COOH, C═O, | — | 92.4 | — | 7.6 | — | — |
| Oxidized | C—OH | | | | | | |
| H₂N—C₂H₄— | —CONHC₂H₄NH₂ | — | 99.10 | 0.90 | — | — | — |
| NH₂ | —CONHC₂H₄N═ | — | 97.41 | 0.91 | — | — | 1.68 |
| | OC₆F₅ | | | | | | |

### EXAMPLE 30

### Preparation of Silyl Derivative from Carboxylic Acid-Functionalized Fibrils

Acid functionalized fibrils prepared as in Example 14 are slurried in dioxane in an inert atmosphere. With stirring, a stoichiometric amount of chlorotriethyl silane is added and reacted for 0.5 hr, after which several drops of a 5% solution of DABCO in dioxane is added. The system is reacted for an additional hour, after which the fibrils are collected by filtration and washed in dioxane. The fibrils are dried at 100° C. in 5″ vacuum overnight.

Table VI summarizes the secondary derivative preparations. Products are analyzed by ESCA. The analysis confirms the incorporation of the desired pendant groups. The products are analyzed by ESCA for C, O, N, Si and F surface contents.

TABLE VI
Summary of Secondary Derivative Preparations

| | | ESCA ANALYSIS, ATOM % | | | | | |
|---|---|---|---|---|---|---|---|
| REACTANT | PENDANT GROUP | S | C | N | O | Si | F |

| | | |
|---|---|---|
| CF₃CH₂OH | —COOCH₂CF3 | NOT ANALYZED |
| PolyEG-600 | —CO—(OC₂H₄O—)H | NOT ANALYZED |
| HO—C₂H₄—SH | —COOC₂H4SH | |
| Cl—SiEt₃ | —COSiEt₃ | |

### EXAMPLE 31

### Preparation of Tertiary and Quaternary Amine Derivatives from Carboxylic Acid Functionalized Fibrils

Tertiary and quaternary amine functional groups can be attached to the surface of carbon nanotubes via an amide or ester bond via a carboxyl group on the nanotube and either an amine or hydroxyl group of the tertiary or quaternary amine precursor. Such tertiary or quaternary amine fibrils are useful as chromatographic matrices for the separation of biomolecules. The tertiary or quaternary amine fibrils can be fabricated into disk-shaped mats or mixed with conventional chromatographic media (such as agarose) for separation purposes.

Preparation of Triethylethanolamine Iodide Precursor

In a 100 ml round bottom flask, 10 g N,N-diethylethanolamine (85.3 mmole) was mixed with 10 ml anhydrous methanol. A mixture of 20 g ethyl iodide (127.95 mmole) and 10 ml anhydrous methanol was then added dropwise using a pipette. The reaction mixture was refluxed for 30 minutes. White crystalline product formed when the reaction mixture was allowed to cool to room temperature. The white solid product was collected by filtration and washed with anhydrous methanol. The product was further dried overnight in a desiccator under vacuum. Product (10.3 g, 37.7 mmole) was obtained in a yield of 33%.

Preparation of Quaternary Amine Functionalized Graphite Fibrils

In a vacuum dried 25 ml Wheaton disposable scintillation vial, 100 mg dry carboxyl fibril (about 0.7 mmole COOH per gram of fibrils) was mixed with 2 ml anhydrous dimethylformamide and the mixture was sonicated for 60 seconds. Two more milliliters of dimethylformamide, 39 mg dimethyl-aminopyridine (0.316 mmole), and 50 μl diisopropylcarbodiimide (0.316 mmole) were added to the reaction vial. The reaction mixture was stirred for one hour at room temperature, then 88 mg triethylethanolamine iodide (0.316 mmole) was added to the vial and the reaction was allowed to go overnight. The resulting fibrils were washed three times with 20 ml dimethylformamide, three times with 20 ml methylene chloride, three times with 20 ml methanol and finally three times with de-ionized water. The product was dried under vacuum. Results from an elemental analysis of nitrogen showed that about 50% of the carboxyl groups on the fibril had reacted with the primary amino group in the quaternary amine moiety.

### EXAMPLE 32

### Chromatography of Bovine Serum Albumin (BSA) on Tertiary Amine Functionalized Graphite Fibrils.

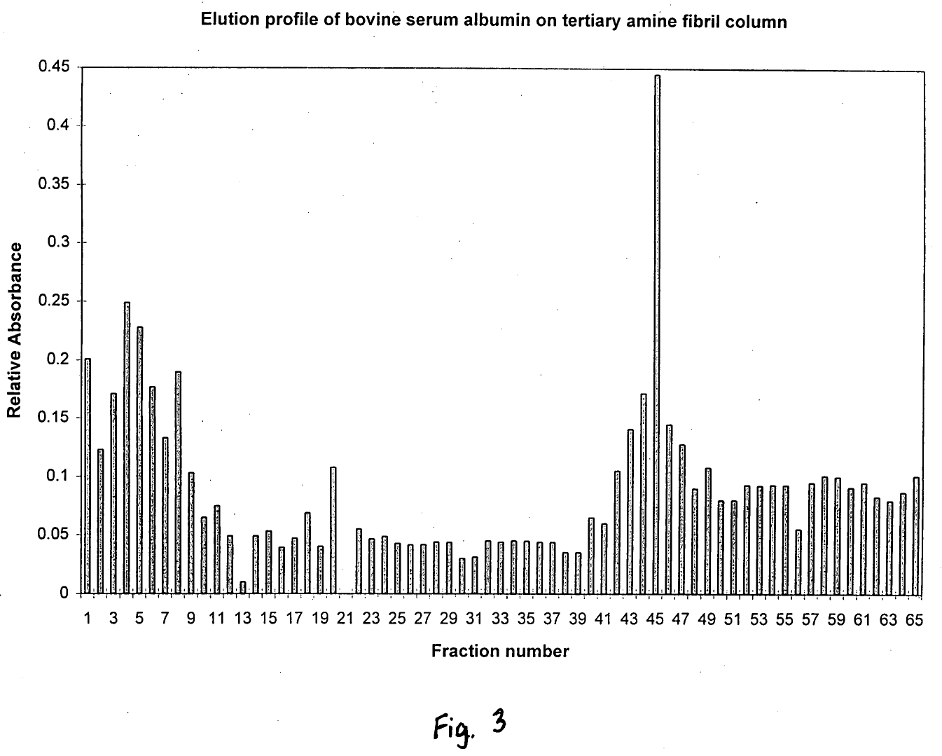

An aqueous slurry containing 60 mg 2-diethylamino ethylamine modified carboxyl fibrils and 180 mg Sephadex G-25 superfine resin (Pharmacia, Uppsala, Sweden) was allowed to stand overnight at room temperature to ensure full hydration of the solid support. The slurry was packed into a 1 cm×3.5 cm column. The column was equilibrated with 5 mM sodium phosphate buffer (pH 7.3) at a flow rate of 0.2 ml/min. BSA (0.6 mg in 0.1 ml de-ionized water) was loaded on the column. The column was eluted with 5 mM sodium phosphate at a flow rate of 0.2 ml/min and 0.6 ml fractions were collected. The elution profile was monitored using a UV-visible detector, and is shown in FIG. 3. Once the detector indicated that no more protein was eluting from the column, bound BSA was eluted by adding 1 M KCl in 5 mM sodium phosphate (pH 7.3). The presence of the protein in each fraction was identified by micro BCA assay (Pierce, Rockford, Ill.).

### EXAMPLE 33

### Chromatography of Bovine serum Albumin (BSA) on Quaternary Amine Functionalized Graphite Fibrils.

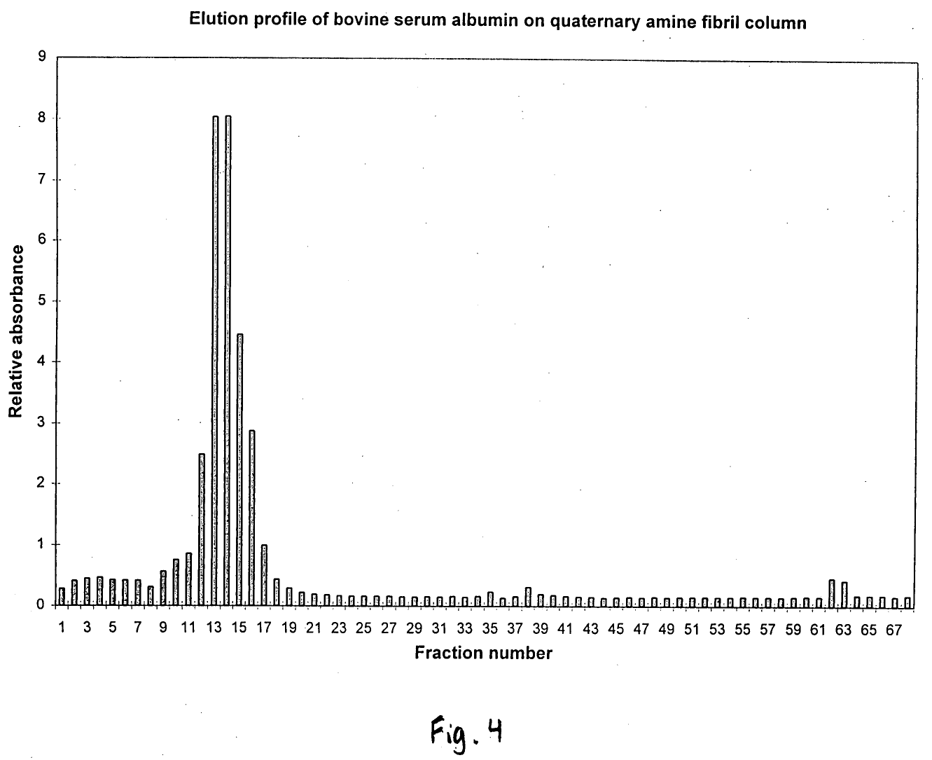

An aqueous slurry containing 100 mg 2-(2-triethylamino ethoxy)ethanol modified carboxyl fibril and 300 mg Sephadex G-25 superfine resin was allowed to stand overnight at room temperature. The resulting slurry was used to pack a 1 cm diameter column. The column was equilibrated with 5 mM sodium phosphate buffer (pH 7.3) at a flow rate of 0.1-0.6 ml/min. BSA (2.7 mg in 0.2 ml de-ionized water) was loaded on the column. The column was eluted with 5 mM sodium phosphate at a flow rate of 0.2 ml/min and 0.6 ml fractions were collected. The elution profile was monitored using a UV-visible detector (FIG. 4). Once the detector indicated that protein was no longer being eluted with 5 mM sodium phosphate buffer, the solvent was changed to 1 M KCl in 5 mM sodium phosphate (pH 7.3). The presence of the protein in each fraction was identified by micro BCA assay (Pierce, Rockford, Ill.).

### 9. Enzymatic Functionalization of Graphitic Carbon

Biocatalysts can be used to introduce functional groups onto the surface of graphitic carbon, especially carbon nanotubes. Until now, graphitic carbon has been modified by purely chemical means (see e.g., U.S. application Ser. No. 08/352,400, filed Dec. 8, 1994). These chemical methods have drawbacks of: (1) harshness of conditions (use of extreme temperatures, extreme acidity or toxic chemicals), and (2) lack of specificity (e.g., oxidation can introduce COOH, COH, and CHO groups). Aqueous suspensions of solid graphitic carbon (such as carbon fibrils; Hyperion, Inc.) are made containing one or more enzymes that are capable of accepting the graphitic carbon as a substrate and performing a chemical reaction resulting in chemically-modified graphitic carbon. The aqueous suspension is maintained at conditions acceptable for the enzyme(s) to carry out the reaction (temperature, pH, salt concentration, etc.) for a time sufficient for the enzyme(s) to catalytically modify the surface of the graphitic carbon. During the reaction, the suspension is continually mixed to allow the enzyme(s) access to the surface of the graphitic carbon. Following a reaction time acceptable for the reaction to proceed to a satisfactory degree, the enzyme is removed from the carbon by filtration washing.

To date two types of enzymes have been used: cytochrome p450 enzymes and peroxidase enzymes. In both cases, the types of enzymes have been well-studied, they accept aromatic type substrates, and their optimal reaction conditions have been worked out. Both enzyme types introduce hydroxyl groups into their substrates and may introduce hydroxyl groups into graphitic carbon. Besides enzymes, other biocatalysts such as ribozymes and catalytic antibodies, or non-biological mimics of enzymes, could be designed to catalytically functionalize carbon nanotubes.

### EXAMPLE 34

### Enzymatic Functionalization Using Rat Liver Microsomes

Cytochrome p450 enzymes are generally believed to function in the liver as detoxifying agents (F. Peter Guengerich, American Scientist, 81, 440-447 and F. Peter Guengerich, J. Biol. Chem., 266, 10019-10022). They hydroxylate foreign compounds such as polyaromatic toxic compounds. Hydroxylation allows these compounds to become water soluble so that they can be eliminated from the body via the urine. There are many different cytochrome p450 enzymes in the liver, each with different substrate specificities. These broad range of specificities is believed to be important because of the wide range of environmental toxins whose detoxification is required. Although individual cytochrome p450s are commercially available, no information is available regarding whether any of these would accept carbon nanotubes as a substrate. Because of this uncertainty, we decided to initially incubate carbon nanotubes with a rat liver extract which contained many different cytochrome p450s.

Two rats (“experimental” rats) were administered phenobarbital (1 g/L, pH 7.0) in their drinking water for one week to induce expression of cytochrome p450 enzymes. Two other rats (“control” rats) were given water without phenobarbital. The rats were then sacrificed and cytochrome p450-containing microsomes were prepared from their livers by standard procedures (see for example, Methods in Enzymology, Vol. 206).

The microsomes were mixed with carbon nanotubes (fibrils) to allow the cytochrome p450s to react with the graphitic carbon. In these experiments, 5 mg of fibrils (both “plain” or nonfunctionalized and “COOH” or oxidized fibrils) were mixed with microsomes (both experimental and control microsomes) in a buffered solution containing 0.1 M Tris, 1.0 mM NADPH, 0.01% NaN₃, 10 mM glucose-6-phosphate, glucose-6-phosphate dehydrogenase (1 unit/mL), pH 7.4. NADPH was included as a co-substrate for cytochrome p450s and glucose-6-phosphate, glucose-6-phosphate dehydrogenase were added to regenerate NADPH from NADP⁺ (if NADP⁺ is generated by cytochrome p450s). The mixtures were rotated at room temperature for about 1.5 days in microcentrifuge tubes. Following the incubation, the fibrils were washed extensively in deionized water, 1 M HCl, 1 M NaOH, 0.05% Triton X-100, 0.05% Tween, methanol, and 1 M NaCl. Following washing, microBCA assay for proteins (Pierce) showed that fibrils seemed to still have protein associated with them (although no protein was detected in the wash solution).

To determine whether hydroxyl groups had been introduced onto the fibril surfaces, the fibrils were reacted with N-FMOC-isoleucine. The different batches of fibrils (control and experimental) (1.5 mg each) were reacted with 333 microliters of a solution of dry DMF containing 4.45 mg/mL FMOC-isoleucine, 1.54 mg/mL dimethylaminopyridine (DMAP) and 2.6 mg/mL 1,3-dicyclohexylcarbodiimide (DCC). Following reaction for two days (while being continuously rotated), the fibrils were washed with DMF, piperidine, methanol, water, DMF, methanol, methylene chloride (600 microliters of each). This wash sequence was repeated three times. Fibrils were sent to Galbraith Laboratories (Knoxville, Tenn.) for amino acid analysis for isoleucine present. The results were equivocal because many other amino acids were seen in addition to isoleucine, indicating that proteins, peptides, and amino acids present in the rat liver microsomal extracts had not completely washed away from the fibrils. Thus, because of technical difficulties in washing and analysis it could not be determined whether or not cytochrome p450's had functionalized the fibrils.

### EXAMPLE 35

### Fibril Functionalization Using Commercially-Available Recombinant Cytochrome p450 Enzymes

To avoid the impurities associated with using rat liver microsomes as a source of cytochrome p450s, individual cytochrome p450 enzymes were purchased (GENTEST, Woburn, Mass.). Because cytochrome p450 enzymes are only active in association with membranes, these enzymes are supplied as microsomal preparations. Using a reaction procedure similar to that described above, we tested the following cytochrome p450s: CYP1A1 (cat.# M111b), CYP1A2 (cat.# M103c), CYP2B6 (cat.# 110a), CYP3A4 (with reductase, cat.# 107r). MgCl₂ (0.67 mg/mL) was also included in the reaction solution. In this experiment, fibrils were washed with the aid of a Soxhlet apparatus.

Analysis of introduced hydroxyl groups was carried out by reaction of cytochrome p450-reacted, washed fibrils with the colored reagent 3,5-dinitrobenzoic acid (DNBA). Coupling was carried out as described above for N-FMOC-isoleucine. Following reaction with DNBA, the fibrils were washed with DMF and residual (covalently attached) DNBA was hydrolyzed using either 6 M HCl or 46 units/mL pig liver esterase (Sigma). Analysis of liberated DNBA was carried out by HPLC analysis of the supernatant surrounding the fibrils following hydrolytic treatment. HPLC analysis of liberated DNBA was carried out on a Waters HPLC system equipped with a Vydac C18 reversed phase analytical column (cat.# 218TP54) and a linear gradient from deionized water containing 0.1% TFA (solvent A) to acetonitrile containing 0.1% TFA (solvent B).

### EXAMPLE 36

### Functionalization of Fibrils Using Peroxidase

Literature descriptions of peroxidase substrate specificities indicated that carbon nanotubes may be substrates for these enzymes (J. S. Dorick et al., Biochemistry (1986), 25, 2946-2951; D. R. Buhler et al., Arch. Biochem. Biophys. (1961) 92, 424-437; H. S. Mason, Advances in Enzymology, (1957) 19, 79; G. D. Nordblom et al., Arch. Biochem. Biophys. (1976) 175, 524-533). To determine whether peroxidase (hydrogen peroxidase, Type II, Sigma) could introduce hydroxyl groups onto the surface of fibrils, fibrils (11 mg) were mixed in a solution containing 50 mM sodium acetate (1.25 mL, pH 5.0), horseradish peroxidase (200 nM), and dihydroxyfumaric acid (15 mg) was added 5 mg at a time for the first 3 hours of the reaction. The reaction was carried out for a total of 5 hours at 4° C. with intermittent bubbling of gaseous oxygen. Following the reaction, the fibrils were washed with water, 1 N NaOH, methanol, and methylene chloride (200 mL of each). A control reaction was carried out using peroxidase that had been heat inactivated (100° C. for 5 minutes).

For analysis of the extent of peroxidase-catalyzed fibril hydroxylation, fibrils were reacted with t-butyldimethylsilyl chloride (Aldrich) in dry DMF in the presence of imidazole. Following washing of the fibrils, the fibrils were sent to Robertson Microlit Laboratories, Inc (Madison, N.J.) for elemental analysis of silicon incorporated into the fibrils. The results of the analysis were equivocal for the presence of silicon on the surface of the fibrils. It is believed that silicon from glassware used in the experiment was present in small chips in the fibrils submitted for elemental analysis. This resulted in a high level of silicon in both experimental and control samples. The conclusion of the experiment is that peroxidase may have introduced hydroxyl groups into the fibrils but technical difficulties precluded us from determining the presence of any introduced hydroxyl groups.

### 10. Nanotubes Functionalized by Electrophilic Addition to Oxygen-Free Fibril Surfaces or by Metallization

The primary products obtainable by addition of activated electrophiles to oxygen-free fibril surfaces have pendant —COOH, —COCl, —CN, —CH₂NH₂, —CH₂OH, —CH₂-Halogen, or HC═O. These can be converted to secondary derivatives by the following:

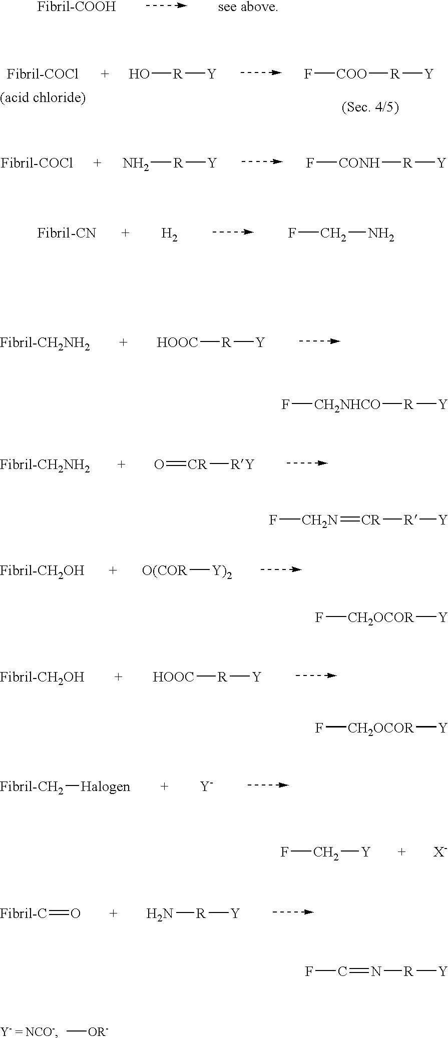

### 11. Dendrimeric Nanotubes

The concentration of functional groups on the surface of nanotubes can be increased by modifying the nanotubes with a series of generations of a polyfunctional reagent that results in the number of the specific functional groups increasing with each generation to form a dendrimer-like structure. The resulting dendrimeric nanotubes are particularly useful as a solid support upon which to covalently immobilize proteins, because they increase the density of protein immobilized on the nanotube surface. The present invention demonstrates that high densities of a specific chemical functionality can be imparted to the surface of high surface area particulate carbon, which has been difficult with previous high surface area carbons.

### EXAMPLE 37

### Preparation of Lysine-Based Dendrimers

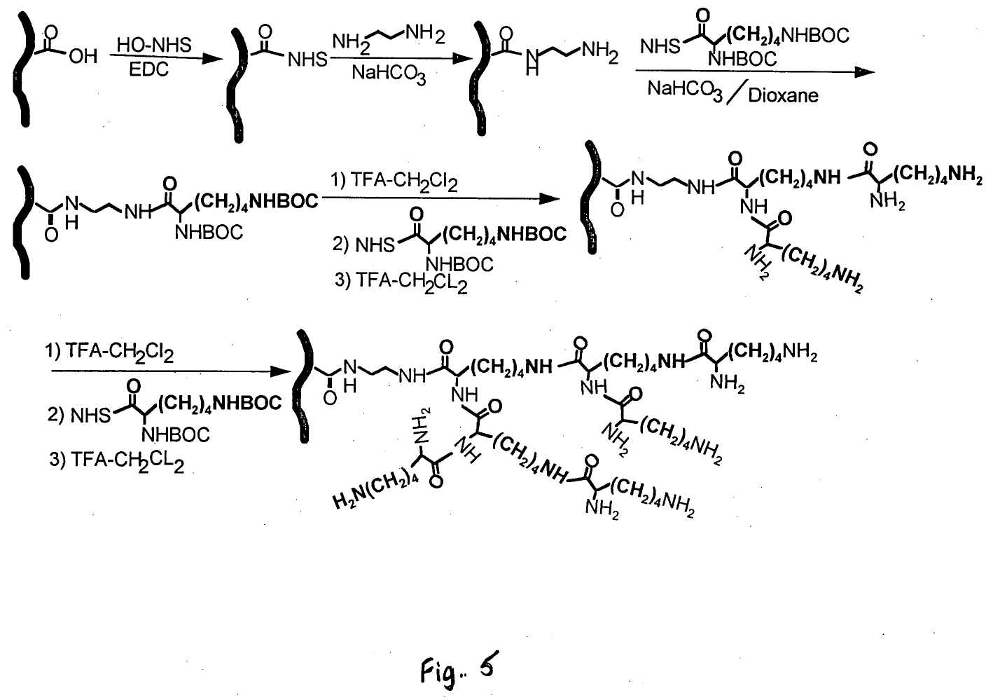

The reaction sequence is shown in FIG. 5.

To a suspension of amino fibrils (90 mg) in sodium bicarbonate (5 ml, 0.2 M, pH 8.6) was added a solution of N_{α},N_{ε}-di-t-boc-L-lysine N-hydroxysuccinimide ester (120 mg, 0.27 mmol) in diosane (5 ml). The reaction mixture was stirred overnight at room temperature. The tert-butoxycarbonyl protected lysine fibrils were extensively washed with water, methanol and methylene chloride and dried under vacuum. The tert-butoxycarbonyl protected lysine fibrils were then treated with trifloroacetic acid (5 ml) in methylene chloride (5 ml) for 2 hours at room temperature. The product amino lysine fibrils were extensively washed with methylene chloride, methanol and water and dried under vacuum. Preparation of the second and the third generation lysine fibrils followed the same procedure. The amino acid analysis data showed that the first generation lysine fibrils contained 0.6 μmols lysine per gram of fibrils, the second generation lysine fibrils contained 1.8 μmols per gram of fibrils, and the third generation lysine had 3.6 μmols lysine per gram of fibrils.

Carboxyl dendrimeric fibrils can be prepared by the same method by using aspartic or glutamic acid with carboxyl fibrils.

### EXAMPLE 38

### Preparation of Carboxylate-Terminated Dendrimers

Carboxylate terminated dendrimers with a carbon nanotube (CN) core are produced by successive, sequential couplings of aminobuty-nitrilotriacetic acid (NTA) and beginning with the NHS ester of chlorate oxidized carbon nanotubes.

Preparation of NTA

NTA was prepared according to the method of Hochuli (E. Hochuli, H. Dobeli, and A. Schacher, J. Chromatography. 411, 177-184 (1987)), the contents of which is hereby incorporated by reference.

Preparation of CN/NHS

CN/NHS were prepared according to the method of Example 20.

Preparation of CN/NTA

0.4 g of NTA•HCl was dissolved in 25 mls of 0.2M NaHCO₃, pH 8.1.1M NaOH was added to bring the pH back up to 7.8. 0.5 g of CN/NHS was added, the mixture was sonicated to disperse the CN and the resultant slurry was left to react for 30 minutes with stirring. The slurry was filtered onto a 0.45 μm nylon membrane and washed 2× with pH 8.1 carbonate buffer and 2× with DI water on filter. The modified CN were twice resuspended in 25 mls of MeOH with sonication, filtered to a solid cake and finally dried in a vacuum desiccator.

Preparation of CN/NTA/NTA

CN/NTA was first converted to the NHS active ester. 0.396 grams of CN/NTA was dried in an oven at 90° C. for 30 minutes and then placed in a 100 ml RB flask with 30 mls of anhydrous dioxane and purged with argon. 0.4 g of N-hydroxysuccinimide added with stirring followed by 0.67 grams of EDC with continued stirring for an additional hour. The CN tended to agglomerate together during this time. The dioxane was decanted off and the solids were washed 2× with 20 mls of anhydrous dioxane. The solids were washed with 20 mls of anhydrous MeOH during which the agglomerates broke up. The solids were filtered onto a 0.45 μm nylon membrane, resuspended in MeOH, filtered and washed on the filter with MeOH.

0.2 g of NTA added to a 50 ml flask and dissolved with 10 drops of 1M NaOH. 20 mls of 0.2M NaHCO₃ at pH 8.1, was added and then all of the CN/NTA/NHS was added and the solution lightly sonicated with a probe sonicator. The mixture was left to react for 2.5 hours at room temperature. The modified CN were filtered onto a 0.45 μm nylon membrane, washed 2× with carbonate buffer, resuspended in DI water with sonication, filtered and washed with DI water. They were then placed in vacuum desiccator to dry.

Preparation of CN/NTA/NTA/NTA

An additional level of NTA was added by following the procedure described above.

Preparation of CN/NTA/NTA/NTA/NTA

An additional level of NTA was added by following the procedure described above.

Samples (approx. 10 mg) of each of the four generation of NTA addition were suspended in 10 mls of DI water with sonication and filtered onto 0.45 μm nylon membranes to form felt-like mats. The mat sections were stored in a vacuum desiccator and analyzed by ESCA for nitrogen (N) to indicate relative amounts of NTA. The results are shown in the table below.

| Material | % N by ESCA |
|---|---|
| CN/NTA | 0 |
| CN/NTA/NTA | 1.45 |
| CN/NTA/NTA/NTA | 1.87 |
| CN/NTA/NTA/NTA/NTA | 2.20 |

The ESCA results verify incorporation of increasing amounts with each successive generation.

### EXAMPLE 39

### Carbon Nanotube Dendrimers as Protein Supports

The density of protein immobilized on carbon nanotubes can be greatly increased by using fibrils derivatized to bear dendrimers. Horseradish peroxidase (HRP) has been immobilized on dendrimeric nanotubes according to the following method:

Plain fibrils (0.49 mg), amino fibrils (0.32 mg), first generation lysine fibrils (0.82 mg), second generation lysine fibrils and third generation lysine fibrils were sonicated with sodium bicarbonate conjugate buffer (600 μl, 0.1 M, containing 0.9% NaCl) for 15 minutes at room temperature. Then they were incubated with HRP solution in sodium bicarbonate conjugate buffer (490 ml, enzyme stock solution of 5.6 mg/ml) for 19 hours at room temperature. The HRP immobilized fibrils were washed with the following buffer (1 ml): 10 mM NaHCO₃ buffer containing 0.9% NaCl at pH 9.5 (1× washing buffer) seven times, 0.1% Triton X-100 in 1× washing buffer five times, 50% ethylene glycol in 1× washing buffer three times. The activity of HRP was assayed with hydrogen peroxide solution (10 μl, 10 mM stock solution) and 2,2-azinobis(3-ethylbenzothiazoline)-6-sulfonic acid diammonium salt (ABTS, 3 μl, mM stock solution) in glycine assay buffer (50 mM, pH 4.4) at 414 nm. The results are shown in the following table:

| Fibrils | nmol HRP/qram fibrils |
|---|---|
| plain Fib | 3.82 |
| Fib-NH₂ | 8.58 |
| Fib-NH-Lys | 28.09 |
| Fib-NH-Lys (Lys)₂ | 28.30 |
| Fib-NH-Lys (Lys)₄ | 46.28 |

### 12. Bifunctional Fibrils

It has been found that more than one type of functional group (e.g. a carboxyl group and an amino group) can be introduced onto a fibril simultaneously by reacting a functionalized nanotube, e.g. a carboxy nanotube, with an amino acid. Such bifunctional fibrils can be used to immobilize multiple molecules, particularly in 1:1 stoichiometries and in close proximity.

### EXAMPLE 40

### Preparation of Bifunctional Fibrils by Addition of Lysine

Synthesis of N_{α}-CBZ-L-lysine Benzyl Ester

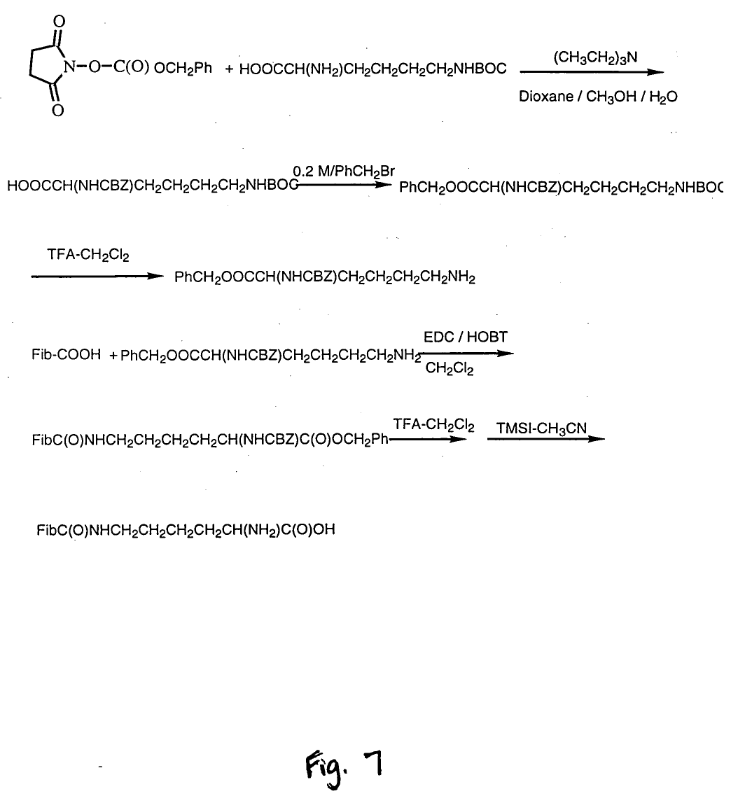

The reaction sequence is shown in FIG. 7. N_{ε}-(tert-butoxycarbonyl)-L-lysine (2 g, 8.12 mmol) was dissolved in methanol (40 ml) and water (40 ml), and the pH was adjusted to 8 with triethylamine. A solution of N-(benzyloxycarbonyl-oxy)succinimide in dioxane (2.4 g, 9.7 mmol in 20 ml) was added to the above mixture and the pH was maintained at 8-9 with triethylamine. The reaction mixture was stirred overnight. The solvent was removed by rotary evaporation to obtain crude N_{α}-CBZ-N_{ε}-(tert-butoxycarbonyl)-L-lysine. N_{α}-CBZ-N_{ε}-(tert-butoxycarbonyl)-L-lysine was treated with 0.2 M calcium carbonate (4 ml) and the aqueous layer was removed to obtain a white solid. The solid was resuspended in N,N-dimethylformamide(40 ml) and benzyl bromide (1.16 ml). The reaction mixture was stirred overnight at room temperature. The reaction mixture was worked up with ethyl acetate and water, and the organic layer was dried over magnesium sulphate. The solvent was removed to obtain crude N_{α}-CBZ-N_{ε}-(tert-butoxycarbonyl)-L-lysine benzyl ester which was purified by silica gel chromatography using 25% hexane in ethyl acetate as a solvent. To N_{α}-CBZ-N_{ε}-(tert-butoxycarbonyl)-L-lysine benzyl ester (1 g, 2.2 mmol) in methylene chloride (10 ml) was added trifluoroacetic acid at 0° C. The reaction mixture was stirred for 10 minutes at 0° C., then stirred for further 2.5 hr at room temperature. The solvent was removed and the crude product was obtained. Pure N_{α}-CBZ-L-lysine benzyl ester was obtained by silica gel chromatography.

Synthesis of N_{α}-CBZ-L-lysine Benzyl Ester Fibrils

To a suspension of carboxyl fibrils (300 mg) in methylene chloride (18 ml) was added a solution of N_{α}-CBZ-L-lysine benzyl ester (148 mg, 0.32 mmol in 20 ml methylene chloride and 176 μl triethylamine). HOBT (43.3 mg, 0.32 mmol) and EDC (61.3 mg, 0.32 mmol) were then added. The reaction mixture was stirred overnight at room temperature to obtain the crude product. The product fibrils were extensively washed with methanol, methylene chloride, and water, then dried under vacuum.

Synthesis of Bifunctional Fibrils Fib-Lys(COOH)NH₂

To N_{α}-CBZ-L-lysine benzyl ester fibrils (113 mg) in methanol (4 ml) was added sodium hydroxide (1 N, 4 ml) and the reaction mixture was stirred overnight. The product N_{α}-CBZ-L-lysine fibrils was extensively washed with water and methanol and the fibrils were dried under vacuum. To a suspension of Nα-CBZ-L-lysine fibrils (50 mg) in acetonitrile (4 ml) was added trimethyl silyl iodide (1 ml). The mixture was stirred for 3 hours at 40° C. The final bifunctional fibrils were extensively washed with water, methanol, 0.5 N sodium hydroxide, acetonitrile and methylene chloride. Amino acid analysis showed 0.3 μmols lysine per gram of fibrils.

Hydroxyl and carboxyl (or amino) bifunctional fibrils can be made by a similar method to that described here by using serine, threonine, or tyrosine. Thiolated and carboxyl (or amino) bifunctional fibrils can be made using cysteine. Carboxyl and amino bifunctional fibrils can be made using aspartic or glutamic acid.

### Uses for Functionalized Nanotubes

Functionalized graphitic nanotubes are useful as solid supports in many biotechnology applications due to their high porosity, chemical and thermal stability and high surface area. They have been found to be compatible with harsh chemical and thermal treatments and very amenable to chemical functionalization.

For example, an enzyme can be covalently immobilized on a modified nanotube while retaining its biological activity. In addition, nanotubes are also suitable for use as affinity chromatographic supports in biomolecular separations. For example, enzyme inhibitors have been prepared on nanotubes in multi-step syntheses such that the immobilized inhibitors were accessible to macromolecules, and reversible specific biological recognition occurred between proteins and modified fibrils.

The hydrophobicity of the nanotube surface is not enough to immobilize high densities of proteins by adsorption. To increase the hydrophobicity of the nanotube surface and to expand the hydrophobic environment from two dimensions to three dimensions, alkyl chains of varying lengths have been coupled to the nanotube surface. Proteins that have been immobilized on alkyl nanotubes by adsorption include trypsin, alkaline phosphatase, lipase and avidin. The enzyme activities of these immobilized proteins are comparable with those of the free enzymes, proven by the catalytic efficiencies toward the hydrolysis of their substrates in aqueous solutions.

In addition, phenyl-alkyl nanotubes, which are alkyl nanotubes with the addition of a phenyl group on the end of the alkyl chain, have also been prepared. This modification introduced an aromatic structure that interacts with the amino acids phenylalanine, tyrosine, and tryptophan in proteins through π-π interactions. The adsorption of alkaline phosphatase and lipase on phenyl-alkyl nanotubes was comparable to the adsorption on C₈-alkyl nanotubes.

Functionalized fibrils have also been found to be useful as solid supports for protein synthesis.

### 1. Functionalized Nanotubes as Solid Supports for Enzymes

### EXAMPLE 41

### Enzyme Immobilization by Adsorption

Preparation of Alkyl Fibrils

Alkyl fibrils were prepared by reacting 10 mg of carboxyl fibrils, which contained approximately 0.007 mmoles of —COOH group (10 mg fibrils×0.7 mmoles-COOH/mg of fibrils=0.007 mmoles), with 0.14 mmoles of alkylamines in 1.5 ml DMF (N,N-dimethylformamide) using 0.14 mmoles of EDC (1-ethyl-3-(3-dimethylaminopropyl)carbodiimide) and 0.14 mmoles of DMAP (4-dimethylaminopyridine). The chemical reaction is as follows:

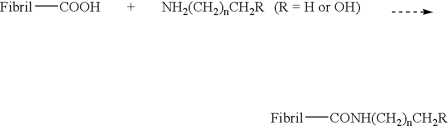

Several different alkyl fibrils with different lengths of alkyl chains (n=5, 7, 9, 17; R═OH only for n=5) were prepared by this procedure. After the reaction was stirred at ambient temperature overnight, fibrils were washed rigorously with 3×25 ml CH₂Cl₂, 3×25 ml MeOH, and 3×25 ml dH₂O. Elemental analysis of the nitrogen content in the fibrils showed that the yields of the reaction were 65-100%.

### Adsorption of Enzymes

The enzymes lipase, trypsin, alkaline phosphatase and avidin were immobilized on the alkyl fibrils of this example by adsorption. The alkyl fibrils and enzyme were mixed at room temperature for three to four hours, followed by washing two to four times with 5 mM sodium phosphate (pH 7.1). Alkaline phosphatase was immobilized on C₈-fibrils and C₆OH-fibrils; trypsin on C₆—, C₈—, C₁₀- and C₁₈-fibrils, lipase on C₆OH—, C₈-, C₁₀- and C₁₈-fibrils, and avidin on C₈-fibrils. The results are shown in the following table:

| Enzyme | μmol/g fibril | mg/g fibril |
|---|---|---|
| lipase | 6.8 | 816 |
| trypsin | 1.7 | 40 |
| alkaline phosphatase | 0.66 | 56 |

| | |
|---|---|
| avidin | not determined |

The kinetic properties of the immobilized enzymes were found to be comparable to those of the free enzymes, as shown in the following table:

| Enzyme | Kₘ (M) | k_{cat} (s⁻¹) | k_{cat}/Kₘ (M⁻¹s⁻ |
|---|---|---|---|
| lipase | 40 × 10⁻⁶ | 0.040 | 0.99 × 10³ |
| lipase-Fibrils | 36 × 10⁻⁶ | 0.048 | 1.34 × 10³ |
| trypsin | 1.2 × 10⁻³ | 4.8 | 4.17 × 10³ |
| trypsin-Fibrils | 7.9 × 10⁻³ | 19.1 | 2.43 × 10³ |

| | | |
|---|---|---|
| substrate: | lipase | 1,2-O-dilauryl-rac-glycero-3-glutaric |
| | | acid resorufin ester |
| | trypsin | N-benzoyl-L-arginine-p-nitroanilide |

### EXAMPLE 42

### Esterification Catalyzed by Fibril-Lipase (Synthesis of Ethyl Butyrate)

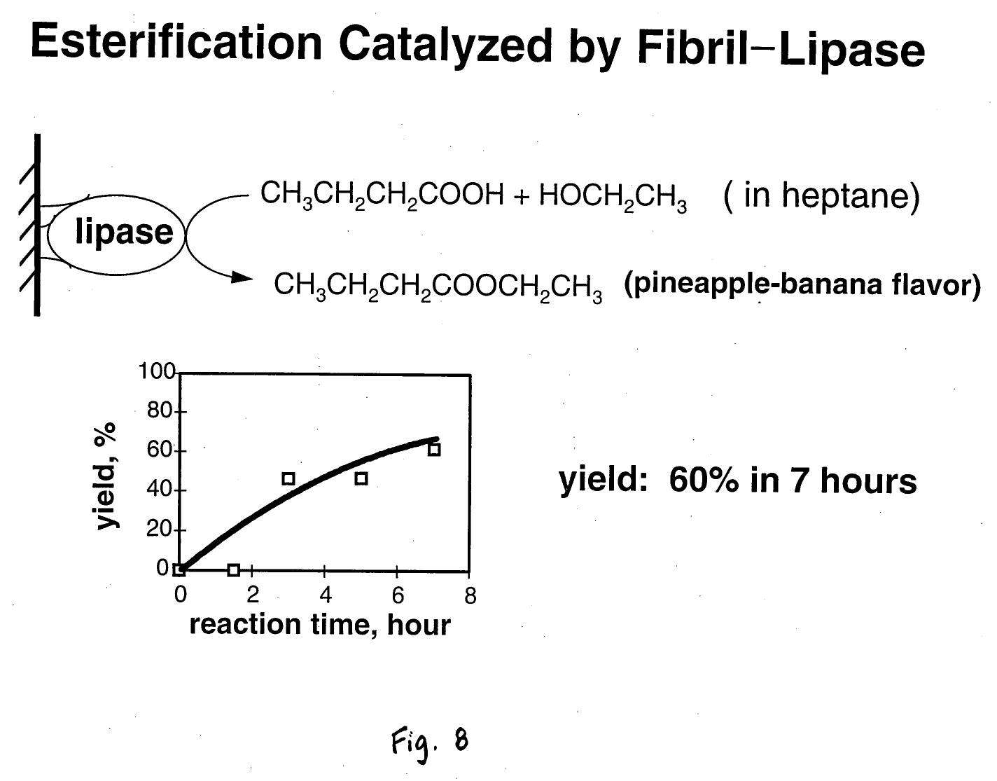

Lipase was immobilized on C₈-alkyl fibrils according to the procedure of Example 41. The lipase fibrils were washed first by dioxane, then a mixture of dioxane and heptane, and finally heptane in order to disperse the fibrils in heptane. To synthesize ethyl butyrate (a food additive which provides pineapple-banana flavor), ethanol (0.4M) and butyric acid (0.25M) were mixed in heptane with 6.2 μm fibril-immobilized lipase. The reaction mixture was stirred at room temperature. The yield was 60% in 7 hours, which was determined by measuring ethanol concentration in the reaction mixture using an established method. The reaction and results are shown in FIG. 8.

### EXAMPLE 43

### Immobilization of Alkaline Phosphatase on Phenyl-akyl Fibrils

Preparation of Phenyl-Alkyl Fibrils

Phenyl-alkyl fibrils were prepared by two different reactions. Reaction 1 mixed 20 mg carboxyl fibrils (containing approximately 0.014 mmoles of —COOH group) with 0.28 mmoles of 4-phenylbutylamine, 0.28 mmoles EDC and 0.28 mmoles DMAP (4-dimethylaminopyridine) in 1.5 ml of DMF (N,N-dimethylformamide). Reaction 2 mixed 20 mg carboxyl fibrils with 0.28 mmoles of 6-phenyl-1-hexanol, 0.28 mmoles DCC (1,3-dicyclohexylcarbodiimide) and 0.28 mmoles DMAP in 1.5 ml of DMF. The reactions were performed at room temperature with stirring overnight. The fibrils were then washed rigorously with 3×25 ml CH₂Cl₂, 3×25 ml MeOH, and 3×25 ml dH₂O.

Preparation of Alkaline Phosphatase-Immobilized Fibrils

0.5 mg of phenyl-alkyl fibrils were suspended in 400 μl of 0.05 M Tris (pH 8.6) and sonicated for 20 minutes. To these fibrils 150 μl of alkaline phosphatase solution (1.67 mg/ml in 5 mM sodium phosphate buffer, pH 7.0) were added and the mixture was rotated at room temperature for 2 hours and stored at 4° C. overnight. The fibrils were then washed with 600 μl of 5 mM sodium phosphate buffer (pH 7.1) twice and suspended in 200 μl of the same buffer.

Quantitation of Specifically Immobilized Alkaline-Phosphatase by Measurement of Catalytic Activity

Alkaline phosphatase reacts with substrate p-nitrophenyl phosphate and releases a color compound that absorbs light at 405 nm with extinction coefficient of 18,200 M⁻¹ cm⁻¹. The assay buffer condition for this reaction was 10 mM Tris, 1 mM MgCl₂ and 0.1 mM ZnCl₂, pH=8.4. The reaction was performed in 1 ml cuvette by mixing 5 μl of p-nitrophenyl phosphate stock solution (0.5 M in 33% DMSO in assay buffer) and 13 μg of alkaline phosphatase fibrils in 1 ml of assay buffer. The absorbance increase of 405 nm was monitored by time scan over 0 minutes. The enzyme activity (μM/min) was then calculated from the initial slope using the extinction coefficient 18200 M⁻¹ cm⁻¹.

For alkaline phosphatase adsorbed on phenyl fibrils from reaction 1, the activity was 6.95 μM/min per 13 μg fibrils. For alkaline phosphatase adsorbed on phenyl fibrils from reaction 2, the activity was 2.58 μM/min per 13 μg fibrils. These results were converted to 0.63 μmoles (or 54 mg) and 0.23 μmoles (or 20 mg) active alkaline phosphatase per gram of fibrils, respectively, by dividing the activity of a known concentration of alkaline phosphatase solution, which was measured to be 879.8 μM/min per 1 μM alkaline phosphatase under the same assay condition.

### EXAMPLE 44

### Immobilization of Lipase on Phenyl Alkyl Fibrils Preparation of Lipase-Immobilized Fibrils

0.5 mg of phenyl-alkyl fibrils were suspended in 50 μl of 5 mM sodium phosphate buffer (pH 7.1) and sonicated for 20 minutes. To these fibrils 350 μl of lipase solution (0.2 mM in 5 mM sodium phosphate buffer, pH 7.1) were added and the mixture was rotated at room temperature for 5 hours and stored at 4° C. overnight. The fibrils were then washed with 600 μl of 5 mM sodium phosphate buffer (pH 7.1) three times and suspended in 200 μl of the same buffer.

Quantitation of Specifically Immobilized Lipase by Measurement of Catalytic Activity

Lipase can react with the substrate 1,2-o-dilauryl-rac-glycero-3-glutaric acid-resorufin ester (Boehringer Mannheim, 1179943) and produce a color compound that absorbs light at 572 nm with extinction coefficient of 60,000 M⁻¹ cm⁻¹. The assay buffer condition for this reaction was 0.1 M KH₂PO₄, pH=6.8. The reaction was performed in 1 ml cuvette by mixing 5 μl of substrate stock solution (7.6 mM in 50% dioxane in Thesit) and 13 μg of alkaline phosphatase fibrils in 1 ml of assay buffer. The absorbance increase at 572 nm was monitored by time scan over 10 minutes. The enzyme activity (μM/min) was then calculated from the initial slope using the extinction coefficient 60,000 M⁻¹ cm⁻¹.

For lipase adsorbed on phenylalkyl fibrils from reaction 1 of Example 43, the activity was 0.078 μM/min per 13 μg fibrils. For lipase adsorbed on phenylalkyl fibrils from reaction 2 of Example 43, the activity was 0.054 μM/min per 13 μg fibrils. These results were converted to 4.7 μmoles (or 564 mg) and 3.3 μmoles (or 396 mg) active lipase per gram of fibrils, respectively, by dividing the activity of a known concentration of lipase solution, which was measured to be 1.3 μM/min per 1 μM lipase under the same assay condition.

### EXAMPLE 45

### Immobilization of Horseradish Peroxidase (HRP) on Amino Alkyl-modified Fibrils

Preparation of Carboxylic Acid-Functionalized Fibrils (Carboxyl Fibrils)

A 10.0 g sample of graphitic fibrils was slurried in 450 mL concentrated H₂SO₄ by mixing with a spatula, then transferred to a reactor flask fitted with inlet/outlets and an overhead stirrer. With stirring and under a slow flow of argon, a charge of 8.68 g of NaClO₃ was added in portions at room temperature over a 24 hour period. Chlorine vapors, which were generated during the entire course of the run, were swept out of the reactor into an aqueous NaOH trap. At the end of the run, the fibril slurry was poured over cracked ice and vacuum filtered. The filter cake was then transferred to a Soxhlet thimble and washed in a Soxhlet extractor with deionized water, exchanging fresh water every several hours. Washing continued until a sample of fibrils, when added to fresh deionized water, did not change the pH of the water. The carboxylated fibrils were then recovered by filtration and dried overnight at 100° C. and 5″ vacuum. The yield was 10.0 g.

Preparation of HRP-Immobilized Fibrils

Amino fibrils made from 1,6-diaminohexane using the method of Example 27 (1.2 mg) were added to conjugation buffer (0.1 M NaHCO₃, 0.9% NaCl, pH 9.5) and the suspension was sonicated for 20 minutes. The fibrils were then washed twice with conjugation buffer in an Eppendorf tube and suspended 430 μL conjugation buffer. A 50-μL aliquot of the suspension (0.14 mg fibrils) was mixed with 4.0 mg activated HRP (Pierce, Rockford, Ill.) dissolved in 50 μL deionized water and the resulting suspension was rotated overnight at 4° C. The HRP-conjugated fibrils were washed extensively in an Eppendorf centrifuge tube with a combination of the following solutions; conjugation buffer, washing buffer (20 mM KH₂PO₄, 0.45% NaCl, pH 6.2), washing buffer containing 0.03-0.1% Triton X-100, and washing buffer containing 50% ethylene glycol. As a control, identical manipulations with activated HRP were carried out with plain (non-derivatized) fibrils, which indicated that the attachment of HRP to amino fibrils was indeed a specific covalent linkage.

Quantitation of Specifically Immobilized HRP by Measurement of Catalytic Activity

Extensive washing removed the majority of non-specifically bound enzyme. Immobilized active HRP was quantitated by substrate turnover using H₂O₂ and the chromogenic substrate 2,2′-azino-bis(3-ethylbenzthiazoline-6-sulfonic acid), diammonium salt (ABTS). Catalytic activity of HRP was spectrophotometrically monitored at 414 nm using 100 μM H₂O₂ and 30 μM ABTS as substrates. The total amount of enzyme bound to amino fibrils in these preliminary studies was 0.0230 μmol HRP/g fibrils. By comparison, control (plain fibrils) nonspecifically bound 0.0048 μmol HRP/g fibrils. By subtraction, the amount of covalently (specifically attached) HRP was 0.0182 μmol/g fibrils.

### EXAMPLE 46

### Affinity Chromatographic Separation of Alkaline Phosphatase (AP) and β-Galactosidase (βG) on Fibrils Bearing Immobilized Enzyme Inhibitors

Preparation of Alkaline Phosphatase Inhibitor Fibrils

Preparation of AP-inhibitor modified fibrils was based on the method of Brenna et al. (1975), Biochem J., 151:291-296. Carboxylated fibrils were used to prepare NHS ester fibrils as described in Example 50 above. NHS ester fibrils (114 mg) were suspended in 4 mL acetone and 10 equivalents (based on the estimation of 0.7 meq NHS ester per gram of fibrils) of tyramine were added. Dry triethylamine (10 equiv.) was added and the mixture was stirred for 3 hours at room temperature. The tyraminyl fibrils were washed under vacuum in a scintered glass funnel first with acetone, then extensively with deionized water.

4-(p-Aminophenylazo)-phenylarsonic acid (66 mg) was suspended in 4 mL of 1 N HCl. The suspension was cooled to 4° C. and mixed slowly with 0.36 mL of 0.5 M NaNO₂. After 15 minutes, the arsonic acid/NaNO₂ mixture was added to the tyraminyl fibrils, which were suspended in 10 mL of 0.1 M NaCO₃ (pH 10.0). The reaction mixture (pH≈10) was stirred overnight at 4° C. The fibrils were then treated with successive washes of 0.1 M Na₂CO₃ (pH 10.0), 8 M guanidine HCl, 25 mM NaOH, and water until the effluent became clear. Atomic absorption analysis of arsenic in the AP-inhibitor fibrils was carried out by Galbraith Laboratories (Knoxville, Tenn.). AP-inhibitor fibrils which contain sidechains containing one atom of arsenic were found by atomic absorption analysis to have any arsenic content of 0.4%. This indicates that roughly 10% of the estimated initial COOH groups were converted to AP-inhibitors in this multi-step synthesis. Based on the surface area of fibrils, this means that there would be one inhibitor molecule (enzyme binding site) for every 500 Å² of surface area.

Preparation of β-Galactosidase-Inhibitor Fibrils.

p-Amino-phenyl-β-D-thiogalactoside (TPEG) derivatized fibrils were prepared based on the method of Ullman, (1984) Gene, 29:27-31. To 8 mg of carboxylated fibrils in 0.2 mL deionized water was added 2.24 mg TPEG. The pH of the suspension was adjusted to 4.0 with 0.1 M HCl and 15 mg EDAC was added. The mixture was stirred for 3 hours at pH 4.0 and room temperature. The reaction was stopped by rapid centrifugation in an Eppendorf tube and removal of the liquid. The β-galactosidase-inhibitor fibrils were washed five-times by repeated resuspension in deionized water and centrifugation.

Affinity Separations

Mixtures of alkaline phosphatase (AP), from E. coli, Type III; Sigma Chemical Co., St. Louis, Mo.) and β-galactosidase (βG) (from E. coli; Calbiochem, La Jolla, Calif.) were separated batchwise on either AP-inhibitor fibrils or βG-inhibitor fibrils in Eppendorf microcentrifuge tubes. For affinity separations, 1.0 mL solutions of loading buffer (20 mM Tris, 10 mM MgCl, 1.6 M NaCl, 10 mM cysteine, pH 7.4) containing both AP (generally approximately 10 units) and βG (generally approximately 280 units) were added to 0.8-1.0 mg of either AP- or βG-inhibitor fibrils. The resulting suspensions were gently vortexed, then rotated at room temperature for 2 hours. Following enzyme binding, the fibrils were sedimented by brief centrifugation in a tabletop centrifuge and the liquid phase containing unbound enzyme was withdrawn and saved for enzyme assay. Washes (7×1.0 mL) with loading buffer were carried out by repeated buffer addition, gentle vortexing, 15-minute rotation, brief centrifugation, and solvent withdrawal with a Pasteur pipette. After the seventh wash, the same manipulations were repeatedly carried out (5×1.0 mL) with the appropriate elution buffer for either βG-inhibitor fibrils (100 mM sodium borate, 10 mM cysteine, 10 mM cysteine, pH 10.0) or AP-inhibitor fibrils (40 mM NaHPO₄, 10 mM Tris, 1.0 mM MgCl₂, 0.1 mM ZnCl₂, pH 8.4).

All fractions (unbound enzyme, washes, and elutions) were assayed for both AP and βG activity. Alkaline phosphatase activity was determined by spectrophotometrically monitoring the rate of hydrolysis of 500 μM p-nitro-phenylphosphate (PNPP) at 410 nm (Δε=18,000 M⁻¹ cm⁻¹). Alkaline phosphatase activity measurements were carried out in 10 mM Tris, 1.0 mM MgCl₂, and 0.1 mM ZnCl₂ at pH 8.4. β-Galactosidase was assayed by spectrophotometrically monitoring the enzyme's ability to hydrolyze 2-nitro-galacto-β-D-pyranoside (ONPG). Initial rates of β-galactosidase-catalyzed hydrolysis of 5.0 mM ONPG were measured at 405 nm (Δε=3500 M⁻¹ cm ⁻¹) in 10 mM Tris, 10 mM MgCl₂, 1.6 M NaCl, 10 mM cysteine, pH 7.4.

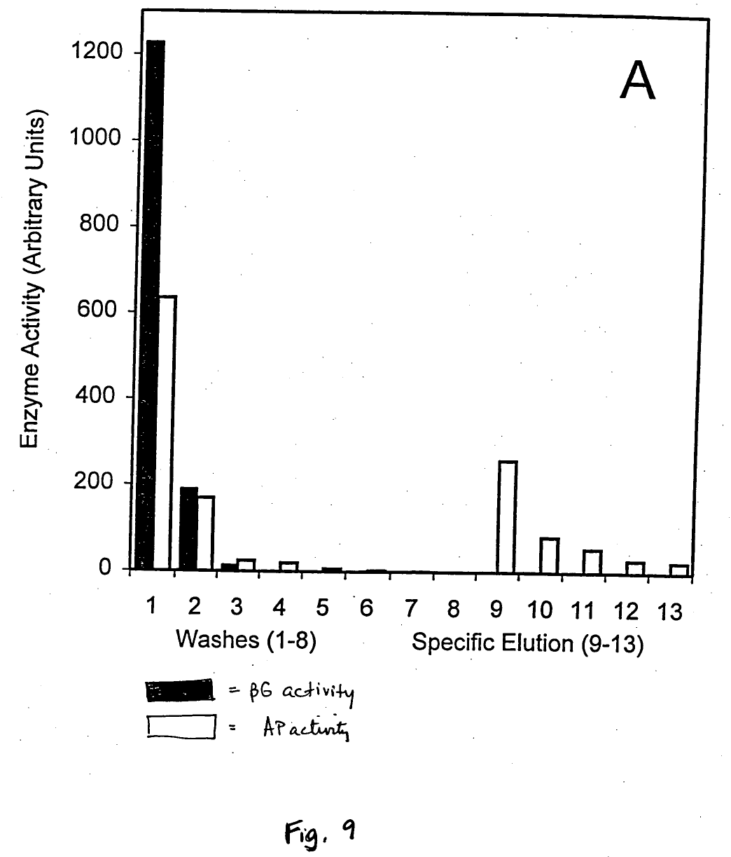

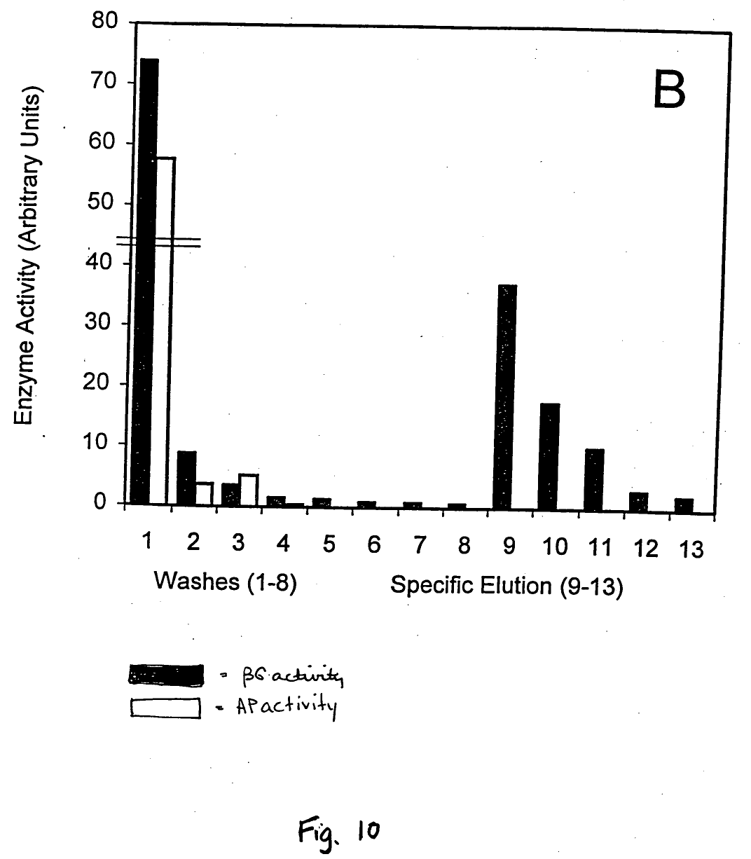

For both AP-inhibitor and βG-inhibitor fibrils, a mixture of AP and βG were added. To facilitate determinations of specific binding capacities, the concentrations of added enzymes were in large excess of the immobilized inhibitor concentrations. For AP-inhibitor fibrils, 0.550 μmol AP/g fibrils was bound (as opposed to non-specific binding of 0.020 μmol βG/g fibrils). For βG-inhibitor fibrils, the capacity was determined to be 0.093 μmol βG/g fibrils (in contrast with non-specific binding of 0.012 μmol AP/g fibrils). The results of the affinity chromatography experiments are shown in FIGS. 9 and 10. AP-inhibitor fibrils did not appreciably bind βG, but bound AP, which specifically eluted when 40 mM phosphate, a competing inhibitor, was added to the buffer (FIG. 9). Fibrils derivatized with βG did not bind substantial amounts of AP, but bound βG, which specifically eluted when the pH was raised to weaken the enzyme-inhibitor association (FIG. 10). These results show that inhibitors were successfully covalently attached to the fibrils, that the immobilized inhibitors were accessible to large molecules, that the inhibitors were available for specific enzyme binding, and when specifically eluted, that the enzymes remained active. In FIG. 10, there appears to be continued leaching of βG from βG-inhibitor fibrils. This may be a result of a natural weak enzyme-inhibitor affinity rather than a shortcoming of the fibrils because the same phenomenon is not seen in FIG. 9 with AP-inhibitor fibrils.

### 2. Functionalized Nanotubes as Solid Supports for Antibodies

It has been found that antibodies can be immobilized on functionalized nanotubes, and that such antibody nanotubes have unique advantages for many applications due to their high surface area per weight, electrical conductivity, and chemical and physical stability. For example, antibody nanotubes can be used as affinity reagents for molecular separations. Antibody nanotubes are also useful for analytical applications, including diagnostic immunoassays such as ECL-based immunoassays.

Antibodies can be immobilized either by covalent binding or non-covalent adsorption. Covalent immobilization was accomplished by various methods; including reductive amination of antibody carbohydrate groups, NHS ester activation of carboxylated fibrils (see Example 27, supra), and reaction of thiolated or maleimido fibrils with reduced or maleimido-modified antibodies (see Examples 23 and 25 supra).

The best method for attaching antibodies to nanotubes will depend on the application they are to be used in. For separations applications, the preferred method may be non-covalent adsorption because the capacity of protein binding seems to be the highest for this method. For methods involving ECL, where the electrical conductivity of the fibrils may be important, covalent methods may be preferred (the alkyl appendages are weak electrical conductors and can be expected to insulate the fibrils). Reductive amination may be the best way to covalently attach antibodies to fibrils because, by using this method, the antibodies are correctly oriented so that their binding sites are pointing outward (away from the fibrils).

### 3. Addition of NAD⁺ To Functionalized Nanotubes

It has been found that cofactors such as NAD⁺ can be added to and used as a solid support for biospecific affinity chromatography of proteins that bind to enzyme cofactors. For example, NAD⁺ fibrils have been used as a solid support for the purification of dehydrogenases. The main advantage of using fibrils is their large amount of accessible surface area. An affinity matrix with high surface area is desirable because of the high potential capacity. The fibrils may either be a loose dispersion or fixed into a column or mat.

### EXAMPLE 47

### Affinity Chromatographic Separation of Dehydrogenases on NAD⁺ Fibrils

Preparation of NAD⁺ Fibrils

Fibrils were oxidized to introduce carboxyl groups according to Examples 14 and 15. To the suspension of fibrils (31 mg) in sodium bicarbonate solution (3 ml, 0.2 M, pH 8.6) was added N⁶-[aminohexyl]carbamoylmethyl)-nicotinamide adenine dinucleotide lithium salt solution (25 mg from Sigma in 5 ml sodium bicarbonate solution). The reaction mixture was stirred overnight at room temperature. The product fibrils were extensively washed with water, N,N-dimethylformamide, and methanol. The elemental analysis data showed that the product fibrils contained 130 mmols of NAD molecules per gram of fibrils by nitrogen analysis and 147 mmols of NAD molecules per gram of fibrils by phosphorus analysis. Other NAD⁺ analogs having linkers terminating in an amino group can be used to prepare NAD⁺ fibrils.

Affinity Separation

The NAD⁺ immobilized fibrils (0.26 mg) and plain fibrils (0.37 mg) were sonicated with 0.1% polyethylene glycol (PEG, MW 1000) in sodium phosphate (1 ml, 0.1 M, at pH 7.1) for 30 minutes at 40° C., then incubated for 30 minutes at 40° C. The fibril suspension was centrifuged and the supernatant were removed. The fibrils were incubated with the mixture of L-lactate dehydrogenase (LDH) in 0.1% PEG (1000) sodium phosphate buffer (250 μl, the ratio of the LDH solution and the 0.1% PEG buffer was 1:1) for 90 minutes at 4° C. Then the mixtures were equilibrated for 30 minutes at room temperature. After the incubation of the fibrils with LDH, the fibrils were washed with 0.1% PEG (1000) in sodium phosphate buffer (5×1000 μl) and every washing took 15 minutes with rotation. The LDH was eluted with a 5 mM solution of NADH in 0.1% PEG (1000) sodium phosphate buffer (5 mM 3×1000 μl). During each elution wash the fibrils were rotated for 15 minutes. The LDH activity in the eluents was assayed by measuring the absorbance change at 340 nm during reduction of pyruvate. The assay mixture contained 0.1% PEG (1000) in sodium phosphate buffer (980 μl), pyruvate (3.3 μl, 100 mM stock solution), and each elution fraction (16.7 μl). The enzyme reaction is shown below:

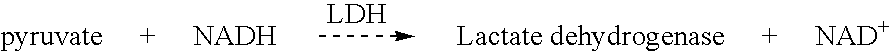

The results showed that the capacity of LDH on the NAD⁺ immobilized fibrils was 484 nmols per gram of fibrils and the capacity of LDH on the plain fibrils (control) was 3.68 nmols per gram of fibrils. The nonspecific binding of LDH was 5.6%.

### 4. Functionalized Nanotubes as Solid Supports for Protein Synthesis

### EXAMPLE 48

### Use of Functionalized Fibrils as solid Support for Peptide Synthesis

To a mixture of amino fibrils (400 mg) and a 4-(hydroxymethyl)-phenoxyacetic acid suspension (255 mg, 1.4 mmol) in methylene chloride (20 ml) were added 1-ethyl-3-(3-dimethylaminopropyl)carbodiimide (EDC, 268 mg, 1.40 mmol) and 1-hydroxybenzotriazole hydrate (HOBT, 189 mg, 1.4 mmol). The reaction mixture was stirred overnight at room temperature under argon gas. The product fibrils were extensively washed with methylene chloride, methanol and water, then dried under vacuum to get fibrils. To the suspension of fibrils in N,N-dimethylformamide (DMF, 2 ml) and methylene chloride (8 ml) were added N-(9-fluorenylmethoxycarbonyl)-O-butyl-L-serine (215 mg, 0.56 mmol), 1,3-dicyclohexylcarbodiimide (DCC, 115 mg, 0.56 mmol) and 4 dimethylaminopyridine (DMAP, 3.4 mg, 0.028 mmol). The reaction mixture was stirred overnight at room temperature and the product fibrils were treated with 20% piperidine in DMF (5×40 ml, each time soaked 1 min.). The product fibrils were then extensively washed with DMF, water, sodium hydroxide (1N), methanol and methylene chloride. The product Fib-Handle-Ser(O+)-COOH (ninhydrin test was positive) was dried under vacuum. For synthesis of dipeptide, the same procedure was used to add arginine. The amino acid analysis data of Fib-Handle-Ser(O+)-Arg(N^{ε}-2,2,5,7,8-pentamethylchroman-6-sulfonyl) shows that it contains 6.5 μmol serine per gram fibrils and 7.6 μmol arginine per gram fibrils. Any other peptide can be made by the same method.

### 5. Biotinylated Fibrils and Biotinylated Alkyl Fibrils

It has been found that fibril surfaces can be functionalized by biotinylation or by both alkylation and biotinylation. The fibrils containing such modifications can then bind any streptavidin conjugated substances such as streptavidin beads and streptavidin enzymes.

Fibrils offer great advantages as solid carriers because of their high surface area. Beads, which can be made strongly magnetic, are extremely useful in separation assays. The biotinylated fibrils described herein combine the advantages of both the fibrils and the beads. The biotinylated alkyl fibrils are an extension of the same concept but exhibit the additional protein adsorption property of alkyl fibrils.

The streptavidin- and biotin-coated fibrils can be used in diagnostics and can be used as capture agents for assays such as electrochemiluminescence assays.

A novel feature of this invention is the combination of two solid carriers on one fibril to create a bifunctional fibril. Moreover, the disclosed process increases the surface area for beads and magnifies fibril magnetization.

### EXAMPLE 49

### Preparation of Biotinylated Fibrils

Biotinylated fibrils were prepared by mixing 2.4 mg of amino fibrils prepared as described in Example 16 and 9 mg of NHS ester long chain biotin in buffer 0.2 M NaHCO₃ at a pH of 8.15. The mixture was rotated at room temperature for four hours and washed with the same buffer twice.

### EXAMPLE 50

### Preparation of Biotinylated Alkyl Fibrils

Biotinylated alkyl fibrils were prepared by a two step reaction. First, 4.25 mg of bifunctional fibrils (containing both amino and carboxyl) and 25 mg of NHS ester long chain biotin were mixed. The fibrils were washed and dried under vacuum.

The second reaction was carried out by mixing 4 mg of biotinylated bifunctional fibrils with 11 mg of EDC (1-ethyl-3-3-dimethylaminopropyl)carbodiimide), 7.5 mg of DMAP (4-dimethylaminopyridine) and 10 μl of NH₂(CH₂)₇CH₃ in 0.5 ml of DMF. The mixture was stirred at room temperature overnight. The final biotinylated alkyl fibrils were washed by CH₂Cl₂, MeOH, and dH₂O

### EXAMPLE 51

### Biotinylated Fibrils as a Solid Support in Assays

Biotinylated fibrils can be used in assays involving formats that require streptavidin-biotin or avidin-biotin interactions. Biotinylated fibrils could, for example, be further derivatized with streptavidin. Biotin covalently linked to fibrils (see Example 50) could form strong non-covalent binding interactions with streptavidin. Because streptavidin is a tetrameric protein with four equivalent binding sites, streptavidin bound to biotinylated fibrils would almost certainly have unoccupied binding sites to which additional biotinylated reagents could bind. Thus, biotinylated fibrils would be converted to streptavidin-coated fibrils.

There are a number of analytical tests that could be performed with such fibril-biotin-streptavidin (FBS) supports. For example, a biotinylated anti-analyte antibody could be captured on the FBS support (either before or after the antibody has complexed to an analyte). Assays using biotinylated anti-analyte antibodies are well established. Such assays include competitive assays where the analyte of interest competes with a labeled analyte for binding to the anti-analyte antibody. Free (unbound) analyte and free (unbound) labeled analyte can be washed from the fibril immobilized antibody. The washing step depends on the fibrils being physically separated from the solution phase by common practices involving centrifugation, filtration, or by attraction to a magnet.

Besides a competition assay, a sandwich type immunoassay could be carried out on FBS supports. Sandwich immunoassays are well known in the field of diagnostics. Such assays involve an analyte being bound simultaneously by two antibodies; a first “primary” antibody which is captured on a solid surface by for example being labeled with biotin, and a “secondary” antibody which is not captured by a solid surface but is labeled with a reporter group. Such a sandwich assay could be carried out using fibrils as a solid capture support whereby the fibrils are captured as described in the previous paragraph. Hence, in such an assay, the fibril would have covalently linked to it biotin, which would be bound to streptavidin, which would in turn be bound to a biotinylated primary antibody, which would be bound to analyte (if present), which would be bound to a labeled secondary antibody.

Similarly, DNA probe assays could be carried out using FBS supports. Biotinylated single stranded DNA can be bound to FBS supports and competitive hybridization can occur between complementary single stranded analyte DNA molecules and complementary labeled oligonucleotides.

Another type of biotinylated fibrils, biotinylated alkylated fibrils, can be used in immunoassays and DNA probe assays. As described in Example 51, bifunctional fibrils can be modified by covalent attachment of biotin to one type of functional group and alkyl chains to the other type of functional group. The resultant alkylated, biotinylated fibrils can be used both in specific association with streptavidin or avidin (via biotin) and also for adsorption of proteins (via the alkyl chains).

Alkyl fibrils could be used in conjunction with other solid supports, such as streptavidin-coated magnetic beads. One advantage of fibrils over such beads is that they have a much higher surface area (per unit weight). Thus, if fibrils could be attached to the outside surface of the magnetic beads, this would dramatically improve the surface area and hence the binding capacity of the beads. It is envisioned that alkylated, biotinylated fibrils could be mixed with streptavidin-coated beads resulting in high affinity streptavidin(bead)-biotin(fibril) interactions and hence fibril-coated beads with an extremely high surface area. Because alkyl fibrils can bind proteins by adsorption, the fibril-coated beads could be further derivatized with adsorbed proteins including streptavidin and antibodies. As described above, streptavidin or antibody coated fibrils can be used in immunoassays and DNA probe assays. Thus, fibril-coated beads could improve the properties of the beads by dramatically increasing their surface area such that fewer beads would be required in a given assay to give the same result.

### 6. 3-Dimensional Structures

The oxidized fibrils are more easily dispersed in aqueous media than unoxidized fibrils. Stable, porous 3-dimensional structures with meso- and macropores (pores >2 nm) are very useful as catalysts or chromatography supports. Since fibrils can be dispersed on an individualized basis, a well-dispersed sample which is stabilized by cross-links allows one to construct such a support. Functionalized fibrils are ideal for this application since they are easily dispersed in aqueous or polar media and the functionality provides cross-link points. Additionally, the functionality provides points to support the catalytic or chromatographic sites. The end result is a rigid, 3-dimensional structure with its total surface area accessible with functional sites on which to support the active agent.

Typical applications for these supports in catalysis include their use as a highly porous support for metal catalysts laid down by impregnation, e.g., precious metal hydrogenation catalysts. Moreover, the ability to anchor molecular catalysts by tether to the support via the functionality combined with the very high porosity of the structure allows one to carry out homogeneous reactions in a heterogeneous manner. The tethered molecular catalyst is essentially dangling in a continuous liquid phase, similar to a homogeneous reactor, in which it can make use of the advantages in selectivities and rates that go along with homogeneous reactions. However, being tethered to the solid support allows easy separation and recovery of the active, and in many cases, very expensive catalyst.

These stable, rigid structures also permits carrying out heretofore very difficult reactions, such as asymmetric syntheses or affinity chromatography by attaching a suitable enantiomeric catalyst or selective substrate to the support. Derivatization through Metallo-Pc or Metallo-porphyrin complexes also allows for retrieval of the ligand bonded to the metal ion, and furthermore, any molecule which is bonded to the ligand through the secondary derivatives. For example, in the case where the 3-dimensional structure of functionalized fibrils is an electrode, or part of an electrode, and the functionalization has resulted from adsorption of Co(II)Pc, electrochemical oxidation of CO(II) to Co(III) in the presence of nicotinic acid will produce a non-labile Co(III)-pyridyl complex with a carboxylic acid as the pendent group. Attaching a suitable antigen, antibody, catalytic antibody, or other site-specific trapping agent will permit selective separations of molecules (affinity chromatography) which are otherwise very difficult to achieve. After washing the electrode to remove occluded material, the CO(III) complex containing the target molecule can be electrochemically reduced to recover the labile Co(II) complex. The ligand on Co(II) containing the target molecule can then be recovered by mass action substitution of the labile Co(II) ligand, thereby effecting a separation and recovery of molecules which are otherwise very difficult or expensive to perform (e.g., chiral drugs).

Previously, it was believed that the pores within the functionalized carbon fibril mats were too small to allow significant flow and thus would not be useful as flow through electrodes. There were also problems associated with the use of particulate carbon or other carbon based materials (such as Reticulated Vitreous Carbon (RVC)) as electrode materials. For example, the porous electrode materials could not be formed in situ, packed too densely and formed voids or channels, were subject to dimensional instability during changes in solvent and flow conditions, and were unable to form very thin electrodes. The use of functionalized carbon fibrils as electrodes in a flow cell solved such problems.

The functionalized carbon fibrils used as electrodes in a flow cell can be modified by surface treatment with electroactive agents. The fibrils can also be modified with non-electroactive materials that may serve a catalytic or electrocatalytic function or serve to inhibit unwanted reactions or adsorption of materials from the flowing stream.

These flow through electrodes are useful in separation techniques such as electrochromatography, electrochemically modulated affinity chromatography, electrosynthesis or electrochemically modulated ion exchange chromatography. They can also be used in diagnostic devices that separate and/or analyze material trapped on the carbon fibril mat.

Composite mats composed of functionalized carbon fibrils and other fibers or particulates can also be used. These fibers or particulates can be added to the suspension to alter the final porosity or conductivity of the carbon fibril mat.

### EXAMPLE 52

### Use of Iron Phthalocyanine Functionalized Fibrils as Electrodes in a Flow Cell

Graphitic fibrils were modified by adsorbing Iron(III)phthalocyanine-bis-pyridine (FePc-2Py) (Aldrich 41,016-0). 0.403 grams of fibrils and 0.130 grams of FePc-2Py were added to 150 mls of absolute EtOH and sonicated with a 450 Watt Branson probe sonicator for 5 min. The resulting slurry was filtered onto a 0.45 μm MSI nylon filter in a 47 mm Millipore membrane vacuum filter manifold, rinsed with water and dried in a vacuum oven overnight at 35° C. The final weight was 0.528 grams, indicating substantial adsorption. A spectrophotometric analysis of the filtrate accounted for the remaining FeP-2Py

5 mgs of the FePc-2Py modified fibrils were dispersed in 10 mls of DI water with sonication. The dispersion was deposited onto a piece of 200 mesh stainless steel (SS) woven screen held in a 25 mm membrane filter manifold and allowed to dry at room temperature. A 0.5 inch diameter disk of the SS screen supported fibril mat was cut using an arch punch.

A electrochemical flow cell was constructed from a 13 mm, plastic, Swinney type membrane filter holder by placing a 13 mm diameter disk of gold mesh (400 mesh, Ladd Industries) on top of the membrane support and making electrical contact to the screen with a platinum wire, insulated with Teflone heat shrink tubing that was fed through the wall of the filter holder for external connection as the working electrode of a three electrode potentiostat circuit. The gold mesh was fixed in place with a minimal amount of epoxy around the outer edge. A strip of gold foil was fashioned into a ring and placed in the bottom, down stream section of the filter holder and connected with an insulated Pt wire lead for connection as the counter electrode of a three electrode potentiostat circuit. A ring of 0.5 mm diameter silver wire, electrochemically oxidized in 1M HCl, was placed in the top section of the filter holder with an insulated lead for connection as the reference electrode.

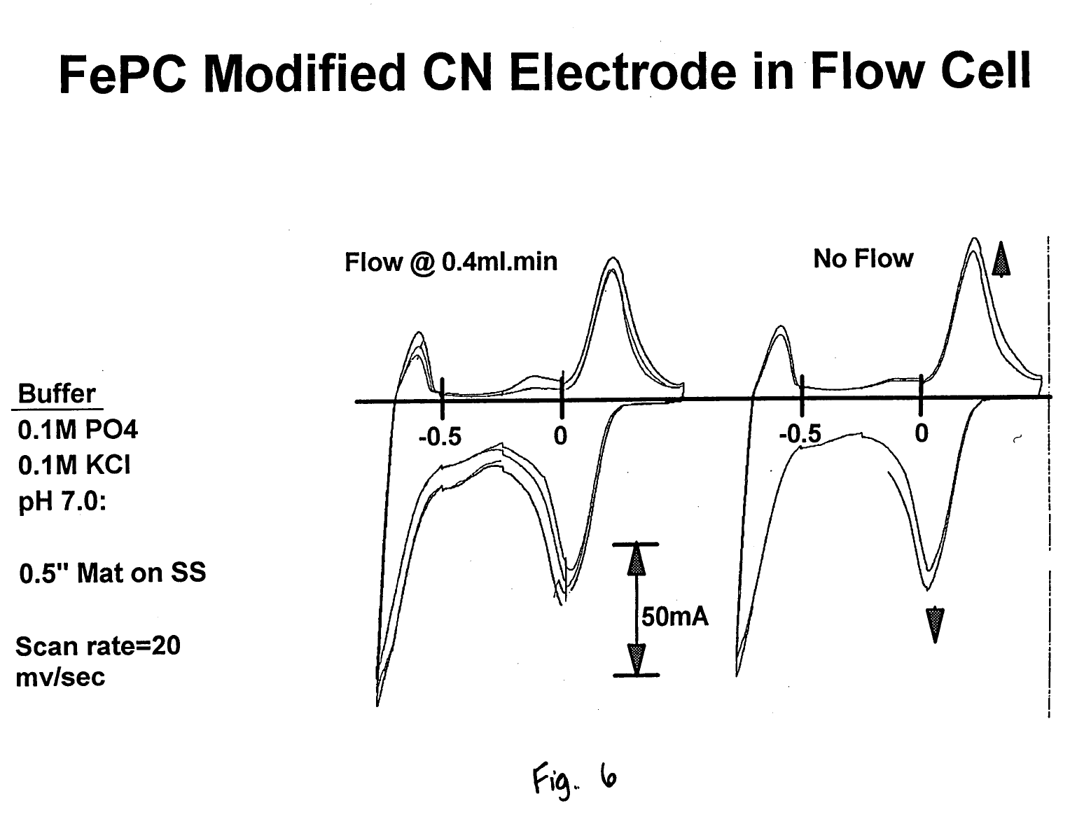

The 0.5 inch diameter disk of FePc-2Py modified CN was placed in the flow cell, which was then connected to the appropriate leads of an EG&G PAR 273 potentiostat. The flow cell was connected to a Sage syringe pump filled with 0.1M KCl in 0.1M potassium phosphate buffer at pH 7.0. Cyclic voltammograms (CVs) were recorded under no flow (static) and flow (0.4 mls/min.) at a potential scan rate of 20 mv/sec. (see FIG. 6). The CVs were nearly identical with and without flow and showed two persistent, reversible oxidation and reduction waves consistent with surface confined FePc-2Py. The persistence of the redox peaks under fluid flow conditions demonstrates that the FePc-2Py is strongly bound to the carbon fibrils and that the use of iron phthalocyanine modified fibrils function well as a flow through electrode material.

Another example of 3-dimensional structures are fibril-ceramic composites.

### EXAMPLE 53

### Preparation of Alumina-Fibril Composites (185-02-01)

One g of nitric acid oxidized fibrils (185-01-02) was highly dispersed in 100 cc DI water using and U/S disintegrator. The fibril slurry was heated to 90° C. and a solution of 0.04 mol aluminum tributoxide dissolved in 20 cc propanol was slowly added. Reflux was continued for 4 hr, after which the condenser was removed to drive out the alcohol. After 30 min the condenser was put back and the slurry refluxed at 100° C. overnight. A black sol with uniform appearance was obtained. The sol was cooled to RT and after one week, a black gel with a smooth surface was formed. The gel was heated at 300° C. in air for 12 hr.

The alumina-fibril composites were examined by SEM. Micrographs of cracked surfaces showed a homogeneous dispersion of fibrils in the gel.

### EXAMPLE 54

### Preparation of Silica-Fibril Composites (173-85-03)

Two g of nitric acid oxidized fibrils (173-83-03) were highly dispersed on 200 cc ethanol using ultrasonification. A solution of 0.1 mol tetraethoxysilane dissolved in 50 cc ethanol was slowly added to the slurry at RT, followed by 3 cc conc. HCL. The mixture was heated to 85° C. and maintained at that temperature until the volume was reduced to 100 cc. The mixture was cooled and set aside until it formed a black solid gel. The gel was heated at 300° C. in air.

The silica-fibril composites were examined by SEM. Micrographs of cracked surfaces showed a homogeneous dispersion of fibrils in the gel.

Similar preparations with other ceramics, such as zirconia, titania, rare earth oxides as well as ternary oxides can be prepared.

### 7. Incorporation of Graphitic Nanotubes onto Polymer Beads

Polymer beads, especially magnetic polymer beads containing an Fe₃O₄ core, such as those manufactured by Dynal and others, have many uses in diagnostics. These beads suffer, however, from having a low surface area compared to that available from nanotubes. Functionalized fibrils can be incorporated onto the surface of beads, which allows the polymer/fibril composites to be used as solid supports for separations or analytical application (e.g., electrochemiluminescence assays, enzyme immobilization).

### EXAMPLE 55

### Attachment of Functionalized Fibrils to Functionalized Beads

7.5 mg of magnetic tosyl-activated Dynabeads M-450 (30 mg/ml) beads (Dynal, Oslo, Norway) were washed three times with 0.1 M sodium phosphate buffer, pH 7.5. Then 0.9 ml of 0.1 M sodium phosphate buffer, pH 8.4 was added to the beads and 0.1 ml of amine fibrils were added. The mixture was allowed to rotate for 16-24 hours at room temperature.

When viewed under the microscope clumps of fibrils with beads on the surface of the fibrils were evident.

As illustrated by the foregoing description and examples, the invention has application in the formulation of a wide variety of functionalized nanotubes and uses therefor.

The terms and expressions which have been employed are used as terms of description and not of limitations, and there is no intention in the use of such terms or expressions of excluding any equivalents of the features shown and described as portions thereof, its being recognized that various modifications are possible within the scope of the invention.

What is claimed is:

1. A composition of matter of the formula
[Rₘ
wherein the carbon atoms, Cₙ, are surface carbons of a substantially cylindrical, graphitic nanotube having a length to diameter ratio of greater than 5 and a diameter of less than 0.5 micron,
n is an integer, L is a number less than 0.1 n, m is a number less than 0.5 n,
each of R is the same and is selected from SO₃H, COOH, NH₂, OH, R′CHOH, CHO, CN, COCl, halide, COSH, SH, COOR′, SR′, SiR′₃, Si(OR′)_{y}R′_{3-y}, Si(O—SiR′₂)OR′, R″, Li, AlR′₂₁ Hg—X, TlZ₂ and Mg—X,
y is an integer equal to or less than 3,
R′ is hydrogen, alkyl, aryl, cycloalkyl, aralkyl, cycloaryl, or poly(alkylether),
R″ is fluoroalkyl, fluoroaryl, fluorocycloalkyl or fluoroaralkyl,
X is a halide, and
Z is carboxylate or trifluoroacetate.

2. A composition of matter of the formula
[Rₘ
wherein the carbon atoms, Cₙ, are surface carbons of a substantially cylindrical, graphitic fibril being substantially free of pyrolytically deposited carbon, the projection of the graphite layers on said fibrils extends for a distance of at least two fibril diameters,
n is an integer, L is a number less than 0.1 n, m is a number less than 0.5 n,
each of R is the same and is selected from SO₃H, COOH, NH₂, OH, R′CHOH, CHO, CN, COCl, halide, COSH, SH, COOR′, SR′, SiR′₃, Si(OR′)_{y}R′_{3-y}, Si(O—SiR′₂)OR′, R″, Li, AlR′₂, Hg—X, TlZ₂ and Mg—X,
y is an integer equal to or less than 3,
R′ is hydrogen, alkyl, aryl, cycloalkyl, aralkyl, cycloaryl, or poly(alkylether),
R″ is fluoroalkyl, fluoroaryl, fluorocycloalkyl or fluoroaralkyl,
X is a halide, and
Z is carboxylate or trifluoroacetate.

3. A composition of matter of the formula
[Rₘ
wherein the carbon atoms, Cₙ, are surface atoms of a fishbone fibril,
n is an integer, L is a number less than 0.1 n, m is a number less than 0.5 n,
each of R is the same and is selected from SO₃H, COOH, NH₂, OH, R′CHOH, CHO, CN, COCl, halide, COSH, SH, COOR′, SR′, SiR′₃, Si(OR′)_{y}R′_{3-y}, Si(O—SiR′₂)OR′, R″, Li, AlR′₂, Hg—X, TlZ₂ and Mg—X.
y is an integer equal to or less than 3,
R′ is hydrogen, alkyl, aryl, cycloalkyl, aralkyl, cycloaryl, or poly(alkylether),
R″ is fluoroalkyl, fluoroaryl, fluorocycloalkyl or fluoroaralkyl,
X is a halide, and
Z is carboxylate or trifluoroacetate.

4. A composition of matter of the formula
[Rₘ
wherein the carbon atoms, Cₙ, are surface carbons of a substantially cylindrical, graphitic nanotube having a length to diameter ratio of greater than 5 and a diameter of less than 0.5 micron,
n is an integer, L is a number less than 0.1 n and m is a number less than 0.5 n,
each of R may be the same or different and is selected from SO₃H, COOH, NH₂, OH, R′CHOH, CHO, CN, COCl, halide, COSH, SH, COOR′, SR′, SiR′₃, Si(OR′)_{y}R′_{3-y}, Si(O—SiR′₂)OR′, R″, Li, AlR′₂, Hg—X, TlZ₂ and Mg—X,
y is an integer equal to or less than 3,
R′ is selected from hydrogen, alkyl, aryl, cycloalkyl, aralkyl, cycloaryl, or poly(alkylether),
R″ is a fluoroalkyl, fluoroaryl, fluorocycloalkyl or fluoroaralkyl,
X is a halide,
Z is carboxylate or trifluoroacetate,
and further provided that where each of R is an oxygen-containing group COOH is not present.

5. A composition of matter of the formula
[Rₘ
wherein the carbon atoms, Cₙ, are surface carbons of a substantially cylindrical, graphitic fibril being substantially free of pyrolytically deposited carbon, the projection of the graphite layers on said fibrils extends for a distance of at least two fibril diameters,
n is an integer, L is a number less than 0.1 n and m is a number less than 0.5 n,
each of R may be the same or different and is selected from SO₃H, COOH, NH₂, OH, R′CHOH, CHO, CN, COCl, halide, COSH, SH, COOR′, SR′, SiR′₃, Si(OR′)_{y}R′_{3-y}, Si(O—SiR′₂)OR′, R″, Li, AlR′₂, Hg—X, TlZ₂ and Mg—X,
y is an integer equal to or less than 3,
R′ is hydrogen, alkyl, aryl, cycloalkyl, aralkyl, cycloaryl, or poly(alkylether),
R″ is a fluoroalkyl, fluoroaryl, fluorocycloalkyl or fluoroaralkyl,
X is a halide,
Z is a carboxylate or trifluoroacetate,
and further provided that where each of R is an oxygen-containing group COOH is not present.

6. A composition of matter of the formula
[Rₘ
wherein the carbon atoms, Cₙ, are surface atoms of a fishbone fibril,
n is an integer, L is a number less than 0.1 n and m is a number less than 0.5 n,
each of R may be the same or different and is selected from SO₃H, COOH, NH₂, OH, R′CHOH, CHO, CN, COCl, halide, COSH, SH, COOR′, SR′, SiR′₃, Si(OR′)_{y}R′_{3-y}, Si(O—SiR′2)OR′, R″, Li, AlR′₂, Hg—X, TlZ₂ and Mg—X,
y is an integer equal to or less than 3,
R′ is hydrogen, alkyl, aryl, cycloalkyl, aralkyl, cycloaryl, or poly(alkylether),
R″ is a fluoroalkyl, fluoroaryl, fluorocycloalkyl or fluoroaralkyl,
X is a halide,
Z is a carboxylate or trifluoroacetate,
and further provided that where each of R is an oxygen-containing group COOH is not present.

7. A composition of matter of the formula
[Aₘ
wherein the carbon atoms, Cₙ, are surface carbons of a substantially cylindrical, graphitic nanotube having a length to diameter ratio of greater than 5 and a diameter of less than 0.1 micron,
n is an integer, L is a number less than 0.1 n, m is a number less than 0.5 n,
each of A is selected from 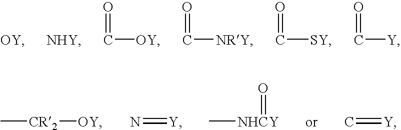
Y is an appropriate functional group of a protein, a peptide, an amino acid, an enzyme, an antibody, a nucleotide, an oligonucleotide, an antigen, or an enzyme substrate, enzyme inhibitor or the transition state analog of an enzyme substrate or is selected from R′—OH, R′—N(R′)₂, R′SH, R′CHO, R′CN, R′X, R′N⁺(R′)₃X⁻, R′SiR′₃, R′Si(OR′_{y}R′_{3-y}, R′Si(O—SiR′₂)OR′, R′—R″, R′—N—CO, (C₂H₄O)_{w}H, (C₃H₆O)_{w}H, (C₂H₄O)_{w}—R′, (C₃H₆O)_{w}—R′, R′, 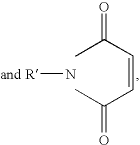
y is an integer equal to or less than 3,
R′ is hydrogen, alkyl, aryl, cycloalkyl, aralkyl or cycloaryl,
R″ is fluoroalkyl, fluoroaryl, fluorocycloalkyl or fluoroaralkyl,
X is a halide,
Z is carboxylate or trifluoroacetate, and
w is an integer greater than one and less than 200.

8. The composition of claim 7 wherein
A is 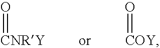
R′ is H and
Y is an amino acid selected from the group consisting of lysine, serine, threonine, tyrosine, aspartic acid and glutamic acid.

9. A composition of matter of the formula
[CₙH_{L}]Aₘ
wherein the carbon atoms, Cₙ, are surface carbons of a substantially cylindrical, graphitic fibril being substantially free of pyrolytically deposited carbon, the projection of the graphite layers on said fibrils extends for a distance of at least two fibril diameters,
n is an integer, L is a number less than 0.1 n, m is a number less than 0.5 n,
each of A is selected from 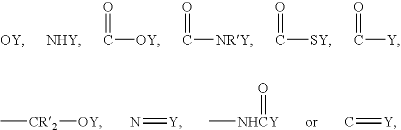
Y is an appropriate functional group of a protein, a peptide, an amino acid, an enzyme, an antibody, a nucleotide, an oligonucleotide, an antigen, or an enzyme substrate, enzyme inhibitor or the transition state analog of an enzyme substrate or is selected from R′—OH, R′—N(R′)₂, R′SH, R′CHO, R′CN, R′X, R′N⁺(R′)₃X⁻, R′SiR′₃, R′Si(OR′)_{y}R′_{3-y}, R′Si)O—SiR′₂)OR′, R′—R″, R′—N—CO, (C₂H₄O)_{w}H, (C₃H₆O)_{w}, (C₂H₄O)_{w}—R′, (c₃H₆O)_{w}—R′, and 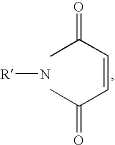
y is an integer equal to or less than 3,
R′ is hydrogen, alkyl, aryl, cycloalkyl, aralkyl or cycloaryl,
R″ is fluoroalkyl, fluoroaryl, fluorocycloalkyl or fluoroaralkyl,
X is a halide,
Z is carboxylate or trifluoroacetate, and
w is an integer greater than one and less than 200.

10. The composition of claim 9 wherein
A is 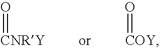
R′ is H and
Y is an amino acid selected from the group consisting of lysine, serine, threonine, tyrosine, aspartic acid and glutamic acid.

11. A composition of matter of the formula
[Aₘ
wherein the carbon atoms, Cₙ, are surface atoms of a fishbone fibril,
n is an integer, L is a number less than 0.1 n, m is a number less than 0.5 n,
each of A is selected from 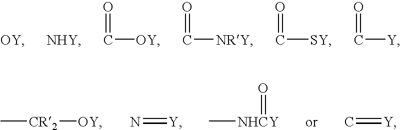
Y is an appropriate functional group of a protein, a peptide, an amino acid, an enzyme, an antibody, a nucleotide, an oligonucleotide, an antigen, or an enzyme substrate, enzyme inhibitor or the transition state analog of an enzyme substrate or is selected from R′—OH, R′—N(R′)₂, R′SH, R′CHO, R′CN, R′X, R′N⁺(R′)₃X⁻, R′SiR′₃, R′Si(OR′)_{y}R′_{3-y}, R′Si(O—SiR′₂)OR′, R′—R″, R′—N—CO, (C₂H₄O)_{w}H, (C₃H₆O)_{w}H, (C₂H₄O)_{w}—R′, (c₃H₆O)_{w}—R′, R′ 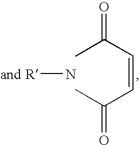
y is an integer equal to or less than 3,
R′ is hydrogen, alkyl, aryl, cycloalkyl, aralkyl or cycloaryl,
R″ is fluoroalkyl, fluoroaryl, fluorocycloalkyl or fluoroaralkyl,
X is a halide,
Z is carboxylate or trifluoroacetate, and
w is an integer greater than one and less than 200.

12. The composition of claim 11 wherein:
A is 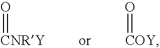
R′ is H, and
Y is an amino acid selected from the group consisting of lysine, serine, threonine, tyrosine, aspartic acid and glutamic acid.

13. A composition of matter of the formula
[[R′-A]ₘ
wherein the carbon atoms, Cₙ, are surface carbons of a substantially cylindrical, graphitic nanotube having a length to diameter ratio of greater than 5 and a diameter of less than 0.5 micron,
n is an integer, L is a number less than 0.1 n, m is a number less than 0.5 n,
each of R′ is alkyl, aryl, cycloalkyl, aralkyl, cycloaryl, or poly(alkylether),
A is selected from 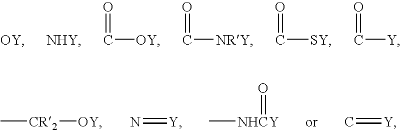
Y is an appropriate functional group of a protein, a peptide, an amino acid, an enzyme, an antibody, a nucleotide, an oligonucleotide, an antigen, or an enzyme substrate, enzyme inhibitor or the transition state analog of an enzyme substrate or is selected from R′—OH, R′—N(R′)₂, R′SH, R′CHO, R′CN, R′X, R′N⁺(R′)₃X⁻, R′SiR′₃, R′Si(OR′)_{y}R′_{3-y}, R′Si(O—SiR′₂)OR′, R′—R″, R′—N—CO, (C₂H₄O)_{w}H, (C₃H₆O)_{w}H, (C₂H₄O)_{w}—R′, (C₃H₆O)_{w}—R′, 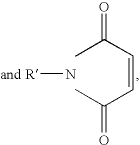
y is an integer equal to or less than 3,
R′ is fluoroalkyl, fluoroaryl, fluorocycloalkyl or fluoroaralkyl,
X is a halide,
Z is carboxylate or trifluoroacetate, and
w is an integer greater than one and less than 200.

14. The composition of claim 13 wherein
A is 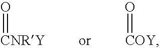
R′ is H, and
Y is an amino acid selected from the group consisting of lysine, serine, threonine, tyrosine, aspartic acid and glutamic acid.

15. A composition of matter of the formula
[[R′-A]ₘ
wherein the carbon atoms, Cₙ, are surface carbons of a substantially cylindrical, graphitic fibril being substantially free of pyrolytically deposited carbon, the projection of the graphite layers on said fibrils extends for a distance of at least two fibril diameters,
n is an integer, L is a number less than 0.1 n, m is a number less than 0.5 n,
each of R′ is alkyl, aryl, cycloalkyl, aralkyl, cycloaryl, or poly(alkylether),
A is selected from 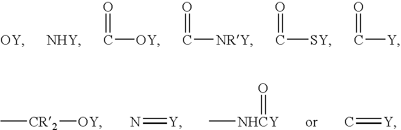
Y is an appropriate functional group of a protein, a peptide, an enzyme, an amino acid, an antibody, a nucleotide, an oligonucleotide, an antigen, or an enzyme substrate, enzyme inhibitor or the transition state analog of an enzyme substrate or is selected from R′—OH, R′—NR′₂, R′SH, R′CHO, R′CN, R′X, R′N⁺(R′)₃X⁻, R′SiR′₃, R′Si(OR′)_{y}R′_{3-y}, R′Si(O—SiR′₂)OR′, R′—R″, R′—N—CO, (C₂H₄O)_{w}H, (C₃H₆O)_{w}H, (C₂H₄O)_{w}—R′, (C₃H₆O)_{w}—R′, R′, 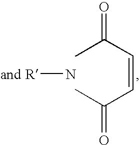
y is an integer equal to or less than 3,
R″ is fluoroalkyl, fluoroaryl, fluorocycloalkyl or fluoroaralkyl,
X is a halide,
Z is carboxylate or trifluoroacetate, and
w is an integer greater than one and less than 200.

16. The composition of claim 15 wherein:
A is 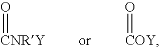
R′ is H, and
Y is an amino acid selected from the group consisting of lysine, serine, threonine, tyrosine, aspartic acid and glutamic acid.

17. A composition of matter of the formula
[[R′-A]ₘ
wherein the carbon atoms, Cₙ, are surface atoms of a fishbone fibril,
n is an integer, L is a number less than 0.1 n, m is a number less than 0.5 n,
each of R′ is alkyl., aryl, cycloalkyl, aralkyl, cycloaryl, or poly(alkyether),
A is selected from 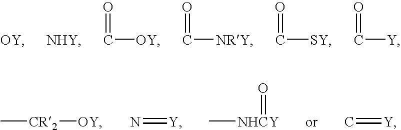
Y is an appropriate functional group of a protein, a peptide, an amino acid, an enzyme, an antibody, a nucleotide, an oligonucleotide, an antigen, or an enzyme substrate, enzyme inhibitor or the transition state analog of an enzyme substrate or is selected from R′—OH, R′—N(R—)₂, R′SH, R′CHO, R′CN, R′X, R′N⁺(R′)₃X⁻, R′SiR′₃, R′Si(OR′)_{y}R′_{3-y}, R′Si(O—SiR′₂)OR′, R′—R″, R′—N—CO, (c₂H₄O)_{w}H, (C₃H₆O)_{w}H, (C₂H₄O)_{w}—R′, (C₃H₆O)_{w}—R′, R′ 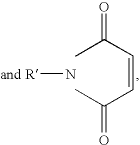
y is an integer equal to or less than 3,
R″ is fluoroalkyl, fluoroaryl, fluorocycloalkyl or fluoroaralkyl,
X is a halide,
Z is carboxylate or trifluoroacetate, and
w is an integer greater than one and less than 200.

18. A composition of matter of the formula
[[X′-Aₐ]ₘ
wherein the carbon atoms, Cₙ, are surface carbons of a substantially cylindrical, graphitic nanotube having a length to diameter ratio of greater than Sand a diameter of less than 0.5 micron,
n is an integer, L is a number less than 0.1 n, m is a number less than 0.5 n, a is an integer less than 10,
each of A is selected from 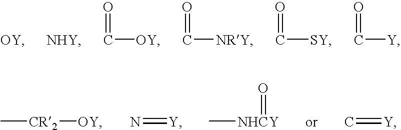
Y is an appropriate functional group of a protein, a peptide, an amino acid, an enzyme, an antibody, a nucleotide, an oligonucleotide, an antigen, or an enzyme substrate, enzyme inhibitor or the transition state analog of an enzyme substrate or is selected from R′—OH, R′—N(R′)₂, R′SH, R′CHO, R′CN, R′X, R′N⁺(R′)₃X⁻, R′Si(OR′)₃, R′Si(OR′)_{y}R′_{3-y}, R′Si(O—SiR′₂)_{y}OR′, R′—R″, R′—N—CO, (C₂H₄O)_{w}H, (C₃H₆O)_{w}H, (C₂H₄O)_{w}—R′, (c₃H₆O)_{w}—R′, R′ 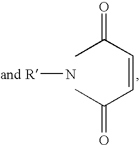
y is an integer equal to or less than 3,
R′ is alkyl, aryl, cycloalkyl, aralkyl or cycloaryl,
R″ is fluoroalkyl, fluoroaryl, fluorocycloalkyl or fluoroaralkyl,
X is a halide,
X′ is a polynuclear aromatic, polyheteronuclear aromatic or metallopolyheteronuclear aromatic moiety,
Z is carboxylate or trifluoroacetate, and
w is an integer greater than one and less than 200.

19. A composition of matter of the formula
[[X′-Aₐ]ₘ
wherein the carbon atoms, Cₙ, are surface carbons of a substantially cylindrical, graphitic fibril being substantially free of pyrolytically deposited carbon, the projection of the graphite layers on said fibrils extends for a distance of at least two fibril diameters,
n is an integer, L is a number less than 0.1 n, m is a number less than 0.5 n, a is an integer less than 10,
each of A is selected from 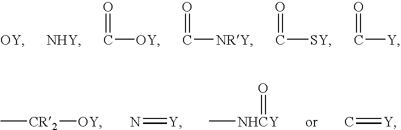
Y is an appropriate functional group of a protein, a peptide, an amino acid, an enzyme, an antibody, a nucleotide, an oligonucleotide, an antigen, or an enzyme substrate, enzyme inhibitor or the transition state analog of an enzyme substrate or is selected from R′—OH, R′—N(R′)₂, R′SH, R′CHO, R′CN, R′X, R′N⁺(R′)₃X⁻, R′SiR′₃, R′Si(OR′)_{y}R′_{3-y}, R′Si(O—SiR′₂)R′, R′—R″, R′—N—CO, (C₂H₄O)_{w}H, (C₃H₆O)_{w}H, (C₂H₄O)_{w}—R′, (C₃H₆O)_{w}—R′, R′ 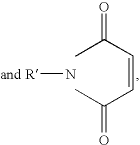
y is an integer equal to or less than 3,
R′ is alkyl, aryl, cycloalkyl, aralkyl or cycloaryl,
R″ is fluoroalkyl, fluoroaryl, fluorocycloalkyl or fluoroaralkyl,
X is a halide,
X′ is a polynuclear aromatic, polyheteronuclear aromatic or metallopolyheteronuclear aromatic moiety,
Z is carboxylate or trifluoroacetate, and
w is an integer greater than one and less than 200.

20. A composition of matter of the formula
[[X′-Aₐ]m
wherein the carbon atoms, Cₙ, are surface atoms of a fishbone fibril,
n is an integer, L is a number less than 0.1 n, m is a number less than 0.5 n, a is an integer less than 10,
each of A is selected from 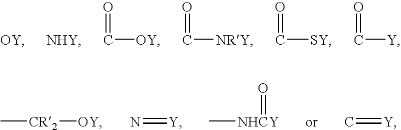
Y is an appropriate functional group of a protein, a peptide, an amino acid, an enzyme, an antibody, a nucleotide, an oligonucleotide, an antigen, or an enzyme substrate, enzyme inhibitor or the transition state analog of an enzyme substrate or is selected from R′O—OH, R′—N(R′)₂, R′SH, R′CHO, R′CN, R′X, R′N⁺(R′)₃X⁻, R′SiR′₃, R′Si(OR′)_{y}R′_{3-y}, R′Si(O—SiR′₂)OR′, R′—R″, R′—N—CO, (C₂H₄O)_{w}H, (C₃H₆O)_{w}H, (C₂H₄O)_{w}—R′, (C₃H₆O)_{w}—R′, R′ 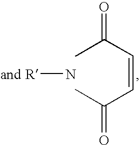
y is an integer equal to or less than 3,
R′ is alkyl, aryl, cycloalkyl, aralkyl or cycloaryl,
R″ is fluoroalkyl, fluoroaryl, fluorocycloalkyl or fluoroaralkyl,
X is a halide,
X′ is a polynuclear aromatic, polyheteronuclear aromatic or metallopolyheternuclear aromatic moiety,
Z is carboxylate or trifluoroacetate, and
w is an integer greater than one and less than 200.

21. A method of forming a composition of matter of the formula
[[CH(R′)OH]ₘ
wherein the carbon atoms, Cₙ, are surface carbons of a substantially cylindrical, graphitic nanotube,
n is an integer, L is a number less than 0.1 n, m is a number less than 0.5 n,
R′ is hydrogen, alkyl, aryl, cycloalkyl, aralkyl, cycloaryl, or poly(alkylether),
comprising the step of reacting the surface carbons with a compound having the formula R′CH₂OH in the presence of a free radical initiator under conditions sufficient to form functionalized nanotubes having the formula [[CH(R′)OH]ₘ.

22. The method of claim 21 wherein said free radical initiator is benzoyl peroxide.

23. A method of forming a composition of matter of the formula
[Aₘ
wherein the carbon atoms, Cₙ, are surface carbons of a substantially cylindrical, graphitic nanotube,
n is an integer, L is a number less than 0.1 n, m is a number less than 0.5 n,
each of A is selected from 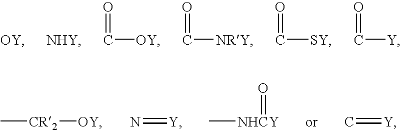
Y is an appropriate functional group of a protein, a peptide, an amino acid, an enzyme, an antibody, an oligonucleotide, a nucleotide, an antigen, or an enzyme substrate, enzyme inhibitor or the transition state analog of an enzyme substrate or is selected from R′—OH, R′—N(R′)₂, R′SH, R′CHO, R′CN, R′X, R′SiR′₃R′—N⁺(R′)₃X⁻, R′—R″, R′—N—CO, (C₂H₄O)_{w}H, (C₃H₆O)_{w}H, (C₂H₄O)_{w}—R′, (C₃H₆O)_{w}—R′, R′ and 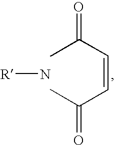
R′ is hydrogen, alkyl, aryl, cycloalkyl, aralkyl or cycloaryl,
R″ is fluoroalkyl, fluoroaryl, fluorocycloalkyl or fluoroaralkyl,
X is a halide,
Z is carboxylate or trifluoroacetate, and
w is an integer greater than one and less than 200, comprising the steps of:
(a) reacting the surface carbons with at least one appropriate reagent under conditions sufficient to form substituted nanotubes having the formula [Rₘ, wherein each of R is the same and is selected from SO₃H, COOH, NH₂, OH, CH(R′)OH, CHO, CN, COCl, halide, COSH, SH, COOR′, SR′, SiR′₃, Si(OR′)_{y}R′_{3-y}, Si(O—SiR′₂)OR′, R″, Li, AlR′₂, Hg—X, TlZ₂ and Mg—X, and y is an integer equal to or less than 3; and
(b) reacting the substituted nanotubes [CₙH_{L}]Rₘ with at least one appropriate reagent under conditions sufficient to form functionalized nanotubes having the formula [Aₘ.

24. A method of forming a composition of matter of the formula
[Aₘ
wherein the carbon atoms, Cₙ, are surface carbons of a substantially cylindrical, graphitic nanotube having a length to diameter ratio of greater than 5 and a diameter of less than 0.1 micron,
n is an integer, L is a number less than 0.1 n, m is a number less than 0.5 n,
each of A is selected from 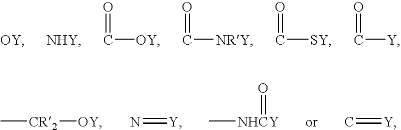
Y is an appropriate functional group of a protein, a peptide, an amino acid, an enzyme, an antibody, an oligonucleotide, a nucleotide, an antigen, or an enzyme substrate, enzyme inhibitor or the transition state analog of an enzyme substrate or is selected from R′—OH, R′—N(R′)₂, R′SH, R′CHO, R′CN, R′X, R′SiR′₃, R′—N⁺(R′)₃X⁻, R′—R″, R′—N—CO, (C₂H₄O)_{w}H, (C₃H₆O)_{w}H, (C₂H₄O)_{w}—R′, (c₃H₆O)_{w}—R′, R′ and 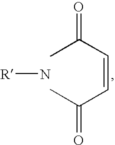
R′ is hydrogen, alkyl, aryl, cycloalkyl, aralkyl or cycloaryl,
R″ is fluoroalkyl, fluoroaryl, fluorocycloalkyl or fluoroaralkyl,
X is a halide,
Z is carboxylate or trifluoroacetate, and
w is an integer greater than one and less than 200, comprising the steps of:
(a) reacting the surface carbons with at least one appropriate reagent under conditions sufficient to form substituted nanotubes having the formula [Rₘ, wherein each of R is selected from SO₃H, COOH, NH₂, OH, CH(R′)OH, CHO, CN, COCl, halide, COSH, SH, COOR′, SR′, SiR′₃, Si(OR′)_{y}R′_{3-y}, Si(O—SiR′₂)OR′, R″, Li, AlR′₂, Hg—X, TlZ₂ and Mg—X, and y is an integer equal to or less than 3; and
(b) reacting the substituted nanotubes [Rₘ with at least one appropriate reagent under conditions sufficient to form functionalized nanotubes having the formula. [Aₘ.

25. A method of forming a composition of matter of the formula
[Aₘ
wherein the carbon atoms, Cₙ, are surface carbons of a substantially cylindrical, graphitic nanotube being substantially free of pyrolytically deposited carbon,
n is an integer, L is a number less than 0.1 n, m is a number less than 0.5 n,
each of A is selected from 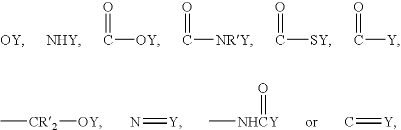
Y is an appropriate functional group of a protein, a peptide, an amino acid, an enzyme, an antibody, an oligonucleotide, a nucleotide, an antigen, or an enzyme substrate, enzyme inhibitor or the transition state analog of an enzyme substrate or is selected from R′—OH, R′—N(R′)₂, R′SH, R′CHO, R′CN, R′X, R′SiR′₃, R′—N⁺(R′)₃X⁻, R′—R″, R′—N—CO, (c₂H₄O)_{w}H, (C₃H₆O)_{w}H, (C₂H₄O)_{w}—R′, (c₃H₆O)_{w}—R′, R′ and 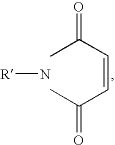
R′ is hydrogen, alkyl, aryl, cycloalkyl, aralkyl or cycloaryl,
R″ is fluoroalkyl, fluoroaryl, fluorocycloalkyl or fluoroaralkyl,
X is a halide,
Z is carboxylate or trifluoroacetate, and
w is an integer greater than one and less than 200, comprising the steps of:
(a) reacting the surface carbons with at least one appropriate reagent under conditions sufficient to form substituted nanotubes having the formula (CₙH_{L}]Rₘ, wherein each of R is selected from SO₃H, COOH, NH₂, OH, CH(R′)OH, CHO, CN, COCl, halide, COSH, SH, COOR′, SR′, SiR′₃, Si(OR′)_{y}R′_{3-y}, Si(O—SiR′₂)OR′, R″, Li, AlR′₂, Hg—X, TlZ₂ and Mg—X, and y is an integer equal to or less than 3; and
(b) reacting the substituted nanotubes (CₙH_{L}]Rₘ with at least one appropriate reagent under conditions sufficient to form functionalized nanotubes having the formula [Aₘ.

26. A method of forming a composition of matter of the formula
[Aₘ
wherein the carbon atoms, Cₙ, are surface carbons of a substantially cylindrical, graphitic nanotube,
n is an integer, L is a number less than 0.1 n, m is a number less than 0.5 n,
each of A is selected from 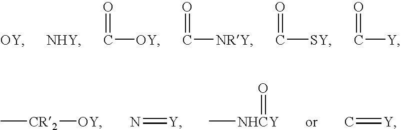
Y is an appropriate functional group of a protein, a peptide, an amino acid, an enzyme, an antibody, an oligonucleotide, a nucleotide, an antigen, or an enzyme substrate, enzyme inhibitor or the transition state analog of an enzyme substrate or is selected from R′—OH, R′—N(R′)₂, R′SH, R′CHO, R′CN, R′X, R′SiR′₃, R′—N⁺(R′)₃X⁻, R′—R″, R′—N—CO, (C₂H₄O)_{w}H, (C₃H₆O)_{w}H, (C₂H₄O)_{w}—R′, (c₃H₆O)_{w}—R′, R′ and 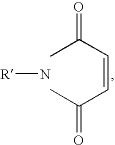
R′ is hydrogen, alkyl, aryl, cycloalkyl, aralkyl or cycloaryl,
R″ is fluoroalkyl, fluoroaryl, fluorocycloalkyl or fluoroaralkyl,
X is a halide,
Z is carboxylate or trifluoroacetate, and
w is an integer greater than one and less than 200, comprising the step of reacting substituted nanotubes [Rₘ with at least one appropriate reagent under conditions sufficient to form functionalized nanotubes having the formula [Aₘ, where each of R is the same and is selected from SO₃H, COOH, NH₂, OH, CH(R′)OH, CHO, CN, COCl, halide, COSH, SH, COOR′, SR′, SiR′₃, Si(OR′)_{y}R′_{3-y}, )Si(O—SiR′₂)OR′, R″, Li, AlR′₂, Hg—X, TlZ₂ and Mg—X, and y is an integer equal to or less than 3.

27. A method of forming a composition of matter of the formula
[Aₘ
wherein the carbon atoms, Cₙ, are surface carbons of a substantially cylindrical, graphitic nanotube having a length to diameter ratio of greater than 5 and a diameter of less than 0.1 micron,
n is an integer, L is a number less than 0.1 n, m is a number less than 0.5 n,
each of A is selected from 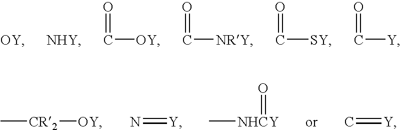
Y is an appropriate functional group of a protein, a peptide, an amino acid, an enzyme, an antibody, an oligonucleotide, a nucleotide, an antigen, or an enzyme substrate, enzyme inhibitor or the transition state analog of an enzyme substrate or is selected from R′—OH, R′—N(R′)₂, R′SH, R′CHO, R′CN, R′X, R′SiR′₃, R—N⁺(R′)₃X⁻, R′—R″, R′—N—CO, (C₂H₄O)_{w}H, (C₃H₆O)_{w}H, (C₂H₄O)_{w}—R′, (C₃H₆O)_{w}—R′, R′ and 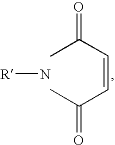
R′ is hydrogen, alkyl, aryl, cycloalkyl, aralkyl or cycloaryl, R″ is fluoroalkyl, fluoroaryl, fluorocycloalkyl or fluoroaralkyl,
X is a halide,
Z is carboxylate or trifluoroacetate, and
w is an integer greater than one and less than 200,
comprising the step of reacting substituted nanotubes [Rₘ with at least one appropriate reagent under conditions sufficient to form functionalized nanotubes having the formula [Aₘ, where each of R is selected from SO₃H, COOH, NH₂, OH, CH(R′)OH, CHO, CN, COCl, halide, COSH, SH, COOR′, SR′, SiR′₃, Si(OR′)_{w}R′_{3-y}, Si(SiR′₂)OR′, R″, Li, AlR′₂, Hg—X, TlZ₂ and Mg—X, and y is an integer equal to or less than 3.

28. A method of forming a composition of matter of the formula
[Aₘ
wherein the carbon atoms, Cₙ, are surface carbons of a substantially cylindrical, graphitic nanotube being substantially free of pyrolytically deposited carbon,
n is an integer, L is a number less than 0.1 n, m is a number less than 0.5 n,
each of A is selected from 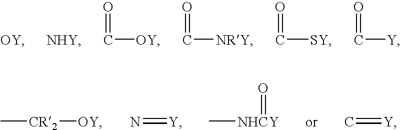
Y is an appropriate functional group of a protein, a peptide, an amino acid, an enzyme, an antibody, an oligonucleotide, a nucleotide, an antigen, or an enzyme substrate, enzyme inhibitor or the transition state analog of an enzyme substrate or is selected from R′—OH, R′—N(R′)₂, R′SH, R′CHO, R′CN, R′X, R′SiR′₃, R′—N⁺(R′)₃X⁻, R′—R″, R′—N—CO, (c₂H₄O)_{w}H, (C₃H₆O)_{w}H, (C₂H₄O)_{w}—R′, (c₃H₆O)_{w}—R′, R′ and 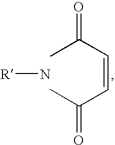
R′ is alkyl, aryl, cycloalkyl, aralkyl or cycloaryl,
R″ is fluoroalkyl, fluoroaryl, fluorocycloalkyl or fluoroaralkyl,
X is a halide,
Z is carboxylate or trifluoroacetate, and
w is an integer greater than one and less than 200,
comprising the step of reacting substituted nanotubes [Rₘ with at least one appropriate reagent under conditions sufficient to form functionalized nanotubes having the formula [Aₘ, where each of R is selected from SO₃H, COOH, NH₂, OH, CH(′R)OH, CHO, CN, COCl, halide, COSH, SH, COOR′, SR′, SiR′₃, Si(OR′)_{y}R′_{3-y}, Si(O—SiR′₂)OR′, R″, Li, AlR′₂, Hg—X, TlZ₂ and Mg—X, and y is an integer equal to or less than 3.

29. A method of forming a composition of matter of the formula
[[R′-A]ₘ
wherein the carbon atoms, Cₙ, are surface carbons of a substantially cylindrical, graphitic nanotube,
n is an integer, L is a number less than 0.4 n, m is a number less than 0.5 n,
R′ is alkyl, aryl, cycloalkyl, aralkyl, cycloaryl, or poly(alkyether),
X is a halide,
each of A is selected from 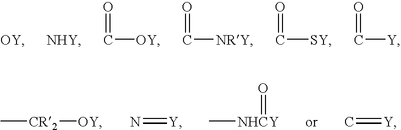
Y is an appropriate functional group of a protein, a peptide, an amino acid, an enzyme, an antibody, an oligonucleotide, a nucleotide, an antigen, or an enzyme substrate, enzyme inhibitor or the transition state analog of an enzyme substrate or is selected from R′—OH, R′—NH₂, R′SH, R′CHO, R′CN, R′X, R′SiR′₃, R′—R″, R′—N—CO, (C₂H₄O)_{w}H, (C₃H₆O)_{w}H, (C₂H₄O)_{w}—R′, (C₃H₆O)_{w}—R′, R′ and 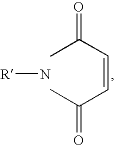
R″ is fluoroalkyl, fluoroaryl, fluorocycloalkyl or fluoroaralkyl, and
Z is carboxylate or trifluoroacetate,
comprising the step of reacting substituted nanotubes having the formula [[R′—R]ₘ with at least one appropriate reagent under conditions sufficient to form functionalized nanotubes having the formula [[R′A]ₘ, where each of R is selected from SO₃H, COOH, NH₂, OH, CH(R′)OH, CHO, CN, COCl, halide, COSH, SH, COOR′, SR′, SiR′₃, Si(OR′)_{y}R′_{3-y}, Si(O—SiR′₂)OR′, R″, Li, AlR′₂, Hg—X, TlZ₂ and Mg—X, and y is an integer equal to or less than 3.

30. A method of forming a composition of matter of the formula
[[X′Rₐ]ₘ
wherein the carbon atoms, Cₙ, are surface carbons of a substantially cylindrical, graphitic nanotube,
n is an integer, L is a number less than 0.1 n, m is a number less than 0.5 n, a is zero or an integer less than 10,
each of R is selected from SO₃H, COOH, NH₂, OH, CH(R′)OH, CHO, CN, COCl, halide, COSH, SH, COOR′, SR′, SiR′₃, Si(OR′)_{y}R′_{3-y}, Si(O—SiR′₂)OR′, R″, Li, AlR′₂, Hg—X, TlZ₂ and Mg—X,
y is an integer equal to or less than 3,
R′ is alkyl, aryl, cycloalkyl, aralkyl or cycloaryl,
X is a halide,
X′ is a polynuclear aromatic, polyheteronuclear aromatic or metallopolyheteronuclear aromatic moiety,
R″ is fluoroalkyl, fluoroaryl, fluorocycloalkyl or fluoroaralkyl, and
Z is carboxylate or trifluoroacetate,
comprising the step of adsorbing at least one appropriate macrocyclic compound onto the surface of the graphitic nanotube under conditions sufficient to form a functionalized nanotube having the formula [[X′—Rₐ]ₘ.

31. A method of forming a composition of matter of the formula
[[X′-Aₐ]ₘ
wherein the carbon atoms, Cₙ, are surface carbons of a substantially cylindrical, graphitic nanotube,
n is an integer, L is a number less than 0.1 n, m is a number less than 0.5 n, a is an integer less than 10,
each of A is selected from 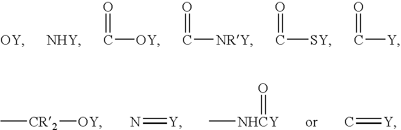
Y is an appropriate functional group of a protein, a peptide, an amino acid, an enzyme, an antibody, an oligonucleotide, a nucleotide, an antigen, or an enzyme substrate, enzyme inhibitor or the transition state analog of an enzyme substrate or is selected from R′—OH, R′—NH₂, R′SH, R′CHO, R′CN, R′X, R′SiR′₃, R′—R″, R′—N—CO, (C₂H₄O)_{w}H, (C₃H₆O)_{w}H, (C₂H₄O)_{w}—R′, (C₃H₆O)_{w}—R′, R′ and 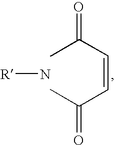
R′ is hydrogen, alkyl, aryl, cycloalkyl, aralkyl or cycloaryl,
R″ is fluoroalkyl, fluoroaryl, fluorocycloalkyl or fluoroaralkyl,
X is a halide,
X′ is a polynuclear aromatic, polyheteronuclear aromatic or metallopolyheteronuclear aromatic moiety,
Z is carboxylate or trifluoroacetate, and
w is an integer greater than one and less than 200, comprising the steps of:
(a) adsorbing at least one appropriate macrocyclic compound onto the surface of the graphitic nanotube under conditions sufficient to form a substituted nanotube having the formula [[X′—Rₐ]ₘ, where each of R is selected from SO₃H, COOH, NH₂, OH, CHO, CN, COCl, halide, COSH, SH, COOR′, SR′, SiR′₃, Si(OR′)_{y}R′_{3-y}, Si(O—SiR′₂—OR′, R″, Li, AlR′₂, Hg—X, TlZ₂ and Mg—X, and y is an integer equal to or less than 3; and
(b) reacting the substituted nanotubes [[X′—Rₐ]ₘ with at least one appropriate reagent under conditions sufficient to form a functionalized nanotube having the formula [[X′-Aₐ]ₘ.

32. A method of forming a composition of matter of the formula
[[X′-Aₐ]ₘ
wherein the carbon atoms, Cₙ, are surface carbons of a substantially cylindrical, graphitic nanotube,
wherein n is an integer, L is a number less than 0.1 n, m is a number less than 0.5 n, a is an integer less than 10,
each of A is selected from 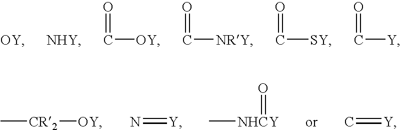
Y is an appropriate functional group of a protein, a peptide, an amino acid, an enzyme, an antibody, an oligonucleotide, a nucleotide, an antigen, or an enzyme substrate, enzyme inhibitor or the transition state analog of an enzyme substrate or is selected from R′—OH, R′—NH₂, R′SH, R′CHO, R′CN, R′X, R′SiR′₃, R′—R″, R′—N—CO, (C₂H₄O)_{w}H, (C₃H₆O)_{w}H (c₂H₄O)_{w}—R′, (C₃H₆O)_{w}—R′, R′ and 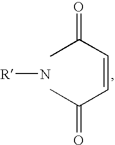
R′ is alkyl, aryl, cycloalkyl, aralkyl or cycloaryl,
R″ is fluoroalkyl, fluoroaryl, fluorocycloalkyl or fluoroaralkyl,
X is a halide,
X′ is a polynuclear aromatic, polyheteronuclear aromatic or metallopolyheteronuclear aromatic moiety,
Z is carboxylate or trifluoroacetate, and
w is an integer greater than one and less than 200,
comprising the step of reacting the substituted nanotubes [[X′—Rₐ]ₘ with at least one appropriate reagent under conditions sufficient to form a functionalized nanotube having the formula [[X′-Aₐ]ₘ, where each of R is selected from SO₃H, COOH, NH₂, OH, CHO, CN, COCl, halide, COSH, SH, COOR′, SR′, SiR′₃, Si(OR′)_{y}R′_{3-y}, Si)O—SiR′₂)OR′, R″, Li, AlR′₂, Hg—X, TlZ₂ and Mg—X, and y is an integer equal to or less than 3.

33. A method for forming a composition of matter of the formula 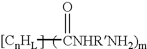
wherein the carbon atoms, Cₙ, are surface carbons of a substantially cylindrical, graphitic nanotube,
n is an integer, L is a number less than 0.1 n and m is a number less that 0.5 n,
R′ is alkyl, aryl, cycloalkyl or cycloaryl,
comprising the steps of:
reacting the surface carbons with at least one appropriate reagent under conditions sufficient to form functionalized nanotubes having the formula [(COOH)ₘ; and
reacting the functionized nanotubes with a compound having two or more amino groups under conditions sufficient to form functionalized nanotubes having the formula

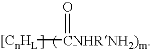

34. A method of forming a composition of matter of the formula
[Rₘ
wherein the carbon atoms, Cₙ, are surface carbons of a substantially cylindrical, graphitic nanotube,
n in an integer, L is a number less than 0.1 n, m is a number less than 0.5 n,
each of R is the same and is selected from SO₃H, COOH, NH₂, OH, CH(R′)OH, CHO, CN, COCl, halide, COSH, SH, COOR′, SR′, SiR′_{3′}, Si(OR′)_{y}R′_{3-y′}, Si(O—SiR′₂)OR′, R″, Li, AlR′₂, Hg—X, TlZ₂ and Mg—X,
y is an integer equal to or less than 3,
R′ is hydrogen, alkyl, aryl, cycloalkyl, aralkyl or cycloaryl,
R″ is fluoroalkyl, fluoroaryl, fluorocycloalkyl or fluoroaralkyl,
X is a halide, and
Z is carboxylate or trifluoroacetate,
comprising the step of reacting the surface carbons with at least one enzyme capable of accepting the nanotube as a substrate and of performing a chemical reaction resulting in a composition of matter of the formula [Rₘ, in aqueous suspension under conditions acceptable for the at least one enzyme to carry out the reaction.

35. The method of claim 34 wherein Rₘ is —OH and the enzyme is a cytochrome p450 enzyme or a peroxidase.

36. A method for forming a composition of matter of the formula
[(NH₂)ₘ
wherein the carbon atoms, Cₙ, are surface carbons of a substantially cylindrical, graphitic nanotube,
n is in an integer, L is a number less than 0.1 n and m is a number less than 0.5 n,
comprising the steps of:
reacting the surface carbons with nitric acid and sulfuric acid to form nitrated nanotubes; and
reducing the nitrated nanotubes to form [(NH₂)ₘ.

37. A method of uniformly substituting the surface of carbon nanotubes with a functional group comprising contacting carbon nanotubes with an effective amount of reactant capable of uniformly substituting a functional group onto the surface of said carbon nanotubes.

38. The method of claim 37, wherein the reactant is a phthalocyanine.

39. The method of claim 38, wherein the reactant is nickel (II) phthalocyaninetetrasulfonic acid (tetrasodium salt) or 1,4,8,11,15,18,22,25-octabutoxy-29H,31H-phthalocyanine.

40. A surface-modified carbon nanotube made by the method comprising contacting carbon nanotube with an effective amount of a reactant for substituting a functional group onto the surface of said carbon nanotube.

41. The surface-modified carbon nanotube of claim 40, wherein the reactant is a phthalocyanine.

42. The surface-modified carbon nanotube of claim 41, wherein the reactant is nickel (II) phthalocyaninetetra-sulfonic acid (tetrasodium salt) or 1,4,8,11,15,18,22,25-octabutoxy-29H,31H-phthalocyanine.

43. A method for linking a protein to a nanotube comprising the steps of:
contacting a nanotube bearing an NHS ester group with a protein under conditions sufficient to form a covalent bond between the NHS ester and the amine group of the protein.

44. An electrode comprising functionalized nanotubes.

45. The electrode of claim 44 wherein the electrode is a porous flow through electrode.

46. An electrode as recited in claim 45, wherein the functionalized nanotubes are phthalocyanine substituted nanotubes.

47. A porous material comprising a multiplicity of functionalized nanotube networks, wherein said functionalized nanotube network comprise at least two functional fibrils linked at functional groups by at least one linker moiety, wherein said linker moiety is either bifunctional or polyfunctional.

48. A method for separating a solute of interest from a sample comprising the steps of:
physically or chemically modifying the surface carbons of a graphitic nanotube with at least one appropriate reagent under conditions sufficient to form functionalized nanotubes;
immobilizing a substance capable of binding the solute of interest on the functionalized nanotubes; and
exposing the substituted nanotubes to the fraction containing the solute of interest under conditions sufficient for the solute of interest to bind the substance immobilized on the functionalized nanotubes.

49. The method of claim 48 wherein the solute of interest is a protein.

50. The method of claim 49, further comprising the step of recovering the functionalized nanotubes.

51. The method of claim 48, wherein the functionalized nanotubes are in the form of a porous mat.

52. The method of claim 48, wherein the functionalized nanotubes are in the form of a packed column.

53. The method of claim 48, wherein the binding is reversible.

54. The method of claim 48, wherein the binding is an ionic interaction.

55. The method of claim 48, wherein the binding is a hydrophobic interaction.

56. The method of claim 48, wherein the binding is through specific molecular recognition.

57. A polymer bead comprising an essentially spherical bead with a diameter of less than 25 Åto which is linked a plurality of functionalized nanotubes.

58. The polymer bead of claim 57 wherein the bead is magnetic.

59. A method for catalyzing a reaction wherein at least one reactant is converted to at least one product comprising the steps of:
physically or chemically modifying the surface carbons of a graphitic nanotube with at least one appropriate reagent under conditions sufficient to form functionalized nanotubes;
immobilizing a biocatalyst capable of catalyzing a reaction on the functionalized nanotubes; and
contacting the functionalized nanotubes with the reactant(s) under conditions sufficient for the reactants(s) to be converted to the product(s).

60. The method of claim 59, further comprising the step of recovering the functionalized nanotubes after the reaction is complete.

61. The method of claim 59 wherein the functionalized nanotubes are in the form of a porous mat.

62. The method of claim 59 wherein the functionalized nanotubes are in the form of a packed column.

63. A method for synthesizing a peptide comprising the step of attaching the terminal amino acid of the peptide to a nanotube via a reversible linker.

64. The method of claim 63 wherein the linker is 4-(hydroxymethyl)phenoxyacetic acid.
